(19) United States
(12) Patent Application Publication
(10) Pub. No.: US 2025/0259738 A1
(43) Pub. Date: Aug. 14, 2025

(54) BUILDING HEALTH, SAFETY, PERFORMANCE SCORING

(71) Applicant: View Operating Corporation, San Jose, CA (US)

(72) Inventors: Justin C. WILSON, Milpitas, CA (US); Piers Iain Ivo Octavian MACNAUGHTON, Milpitas, CA (US); Cory Gordon CLARKE, Milpitas, CA (US)

(21) Appl. No.: 19/086,902

(22) Filed: Mar. 21, 2025

Related U.S. Application Data
(63) Continuation of application No. PCT/US2023/074952, filed on Sep. 22, 2023.
(63) Continuation-in-part of application No. 18/115,694, filed on Feb. 28, 2023, now Pat. No. 12,057,220.
Application No. 18/115,694 is a continuation of application No. 17/328,346, filed on May 24, 2021, now Pat. No. 11,631,493.
(63) Continuation-in-part of application No. 18/263,216, filed on Jul. 27, 2023.
Application No. 18/263,216 is a continuation of application No. PCT/US21/15378, filed on Jan. 28, 2021.
(63) Continuation-in-part of application No. 18/007,047, filed on Jan. 27, 2023.
Application No. 18/007,047 is a continuation-in-part of application No. PCT/US2021/015378, filed on Jan. 28, 2021.
Application No. PCT/US2021/015378 is a continuation-in-part of application No. 17/083,128, filed on Oct. 28, 2020, now abandoned.
Application No. PCT/US2021/043143 is a continuation-in-part of application No. 17/083,128, filed on Oct. 28, 2020, now abandoned.
Application No. 17/083,128 is a continuation-in-part of application No. PCT/US2021/027418, filed on Apr. 15, 2021.
Application No. PCT/US2021/027418 is a continuation-in-part of application No. 17/249,148, filed on Feb. 22, 2021, now Pat. No. 11,735,183.
Application No. 17/249,148 is a continuation-in-part of application No. 16/946,947, filed on Jul. 13, 2020, now Pat. No. 11,592,723.
Application No. 16/946,947 is a continuation-in-part of application No. 16/462,916, filed on May 21, 2019, now Pat. No. 11,137,659.
Application No. PCT/US2021/027418 is a continuation-in-part of application No. 16/950,774, filed on Nov. 17, 2020, now abandoned.
Application No. 16/950,774 is a continuation of application No. 16/608,157, filed on Oct. 24, 2019, now Pat. No. 11,886,089.
Application No. PCT/US2021/027418 is a continuation-in-part of application No. 17/083,128, filed on Oct. 28, 2020, now abandoned.
Application No. 17/083,128 is a continuation-in-part of application No. 17/081,809, filed on Oct. 27, 2020, now Pat. No. 11,460,749.
Application No. 17/081,809 is a continuation of application No. 16/608,159, filed on Oct. 24, 2019, now Pat. No. 11,300,849.
Application No. PCT/US2021/027418 is a continuation-in-part of application No. PCT/US2020/053641, filed on Sep. 30, 2020.
Application No. PCT/US2020/053641 is a continuation-in-part of application No. 16/608,157, filed on Oct. 24, 2019, now Pat. No. 11,886,089.
(60) Provisional application No. 63/376,863, filed on Sep. 23, 2022.
(60) Provisional application No. 63/030,507, filed on May 27, 2020.
(60) Provisional application No. 63/263,806, filed on Nov. 9, 2021.
(60) Provisional application No. 63/080,899, filed on Sep. 21, 2020.
(60) Provisional application No. 63/057,120, filed on Jul. 27, 2020.
(60) Provisional application No. 63/078,805, filed on Sep. 15, 2020.
(60) Provisional application No. 62/967,204, filed on Jan. 29, 2020.
(60) Provisional application No. 63/080,899, filed on Sep. 21, 2020.
(60) Provisional application No. 63/010,977, filed on Apr. 16, 2020.
(60) Provisional application No. 63/052,639, filed on Jul. 16, 2020.
(60) Provisional application No. 63/115,842, filed on Nov. 19, 2020.
(60) Provisional application No. 63/085,254, filed on Sep. 30, 2020.
(60) Provisional application No. 63/170,245, filed on Apr. 2, 2021.
(60) Provisional application No. 63/154,352, filed on Feb. 26, 2021.
(60) Provisional application No. 62/911,271, filed on Oct. 5, 2019.
(60) Provisional application No. 62/952,207, filed on Dec. 20, 2019.
(60) Provisional application No. 62/975,706, filed on Feb. 12, 2020.
(60) Provisional application No. 63/085,254, filed on Sep. 30, 2020.

(51) Int. Cl. G16H 40/20; G01N 33/18; G05B 15/02; G06Q 10/0635; G06Q 10/105; G06Q 50/163; G06Q 50/26; G16H 10/60; G16H 15/00; G16H 50/30

(52) U.S. Cl. CPC G16H 40/20; G01N 33/18; G05B 15/02; G06Q 10/0635; G06Q 10/105; G16H 10/60; G16H 15/00; G16H 50/30; G06Q 50/163; G06Q 50/26

(57) ABSTRACT

Systems and methods of determining composite indices such as, e.g., health, safety, and performance indices, of an enclosure from weighted values of contributing metrics including sensor data and/or controlling one or more building systems based on the composite indices.

### CROSS-REFERENCES TO RELATED APPLICATIONS

This application is a continuation of International Application No. PCT/US2023/74952 designating the United States, filed on Sep. 22, 2023 and claims priority to and benefit of U.S. Provisional Patent Application 63/376,863, filed on Sep. 23, 2022, and titled “BUILDING HEALTH, SAFETY, PERFORMANCE SCORING;” this application is a continuation-in-part of U.S. patent application Ser. No. 18/115,694, filed on Feb. 28, 2023, and titled “SYSTEMS AND METHODS FOR MANAGING BUILDING WELLNESS,” which is a continuation of U.S. patent application Ser. No. 17/328,346, filed on May 24, 2021, and titled “SYSTEMS AND METHODS FOR MANAGING BUILDING WELLNESS,” which claims priority to and benefit of U.S. Provisional Patent Application 63/030,507, filed on May 27, 2020; this application is also related to U.S. patent application Ser. No. 18/263,216, filed on Jul. 27, 2023, and titled “MULTI-SENSOR SYNERGY,” which is a national phase application under 35 U.S.C. § 371 of International PCT Application PCT/US2022/014135, filed on Jan. 28, 2022, and titled “MULTI-SENSOR SYNERGY,” which claims benefit of and priority to U.S. Provisional Patent Application 63/263,806, filed on Nov. 9, 2021 and titled “MULTI-SENSOR SYNERGY;” International PCT Application PCT/US2022/014135 is also a continuation-in-part of International PCT Application PCT/US2021/015378, filed on Jan. 28, 2021, and titled “SENSOR CALIBRATION AND OPERATION;” this application is also a continuation-in-part of U.S. patent application Ser. No. 18/007,047, filed on Jan. 27, 2023, and titled “ATMOSPHERIC ADJUSTMENT IN AN ENCLOSURE,” which is a national phase application under 35 U.S.C. § 371 of International PCT Application PCT/US2021/043143, filed on Jul. 26, 2021, and titled “ATMOSPHERIC ADJUSTMENT IN AN ENCLOSURE;” International PCT Application PCT/US2021/043143 claims benefit of and priority to U.S. Provisional Patent Application 63/080,899, filed on Sep. 21, 2020 and titled “INTERACTION BETWEEN AND ENCLOSURE AND ONE OR MORE OCCUPANTS, U.S. Provisional Patent Application 63/057,120, filed on Jul. 27, 2020 and titled “ATMOSPHERIC ADJUSTMENT IN AN ENCLOSURE,” and U.S. Provisional Patent Application 63/078,805 filed on Sep. 15, 2020 and titled “ATMOSPHERIC ADJUSTMENT IN AN ENCLOSURE;” International PCT Application PCT/US2021/043143 is a continuation-in-part of International PCT Application PCT/US2021/015378, filed on Jan. 28, 2021 and titled “ATMOSPHERIC ADJUSTMENT IN AN ENCLOSURE,” which claims priority to U.S. Provisional Patent Application 62/967,204, filed on Jan. 29, 2020 and titled “SENSOR CALIBRATION AND OPERATION;” International PCT Application PCT/US2021/015378 is a continuation-in-part of U.S. patent application Ser. No. 17/083,128, filed on Oct. 28, 2020 and titled “BUILDING NETWORK,” which is a continuation of U.S. patent application Ser. No. 16/664,089, filed on Oct. 25, 2019 and titled “BUILDING NETWORK,” which is a continuation-in-part of International PCT Application PCT/US2019/030467, filed on May 2, 2019 and titled “EDGE NETWORK FOR BUILDING SERVICES,” which claims priority to U.S. Provisional Patent Application 62/666,033, filed on May 2, 2018 and titled “EDGE NETWORK FOR BUILDING SERVICES;” International PCT Application PCT/US2019/030467 is a continuation-in-part of International PCT Application PCT/US2018/029460, filed on Apr. 25, 2018 and titled “TINTABLE WINDOW SYSTEMS FOR BUILDING SERVICES;” International PCT Application PCT/US2018/029460 claims benefit of and priority to U.S. Provisional Patent Application 62,490,457, filed on Apr. 26, 2017 and titled “ELECTROCHROMIC WINDOWS WITH TRANSPARENT DISPLAY TECHNOLOGY,” U.S. Provisional Patent Application 62,506,514, filed on May 15, 2017 and titled “ELECTROCHROMIC WINDOWS WITH TRANSPARENT DISPLAY TECHNOLOGY,” U.S. Provisional Patent Application 62,507,704, filed on May 17, 2017 and titled “ELECTROCHROMIC WINDOWS WITH TRANSPARENT DISPLAY TECHNOLOGY,” U.S. Provisional Patent Application 62,523,606, filed on Jun. 22, 2017 and titled “ELECTROCHROMIC WINDOWS WITH TRANSPARENT DISPLAY TECHNOLOGY,” and U.S. Provisional Patent Application 62,607,618, filed on Dec. 19, 2017 and titled “ELECTROCHROMIC WINDOWS WITH TRANSPARENT DISPLAY TECHNOLOGY;” U.S. patent application Ser. No. 16/664,089 is also a continuation-in-part of International PCT Application PCT/US2018/029460; International PCT Application PCT/US2021/043143 is also a continuation-in-part of U.S. patent application Ser. No. 17/083,128, filed on Oct. 28, 2021 and titled “BUILDING NETWORK;” International PCT Application PCT/US2021/043143 is also a continuation-in-part of International PCT Application PCT/US2021/027418, filed on Apr. 15, 2021, and titled “INTERACTION BETWEEN AN ENCLOSURE AND ONE OR MORE OCCUPANTS;” International PCT Application PCT/US2021/027418 claims benefit of and priority to U.S. Provisional Patent Application 63/080,899 filed on Sep. 21, 2020, U.S. Provisional Patent Application 63/010,977, filed on Apr. 16, 2020, U.S. Provisional Patent Application 63/052,639, filed on Jul. 16, 2020, U.S. Provisional Patent Application 63/115,842, filed on Nov. 19, 2020, U.S. Provisional Patent Application 63/085,254, filed on Sep. 30, 2020, U.S. Provisional Patent Application 63/170,245, filed on Feb. 4, 2021, and U.S. Provisional Patent Application 63/154,352, filed on Feb. 26, 2021; International PCT Application PCT/US2021/027418 is a continuation-in-part of U.S. patent application Ser. No. 17/249,148, filed on Feb. 22, 2021, which is a continuation of U.S. patent application Ser. No. 16/096,557, filed on Oct. 25, 2018; U.S. patent application Ser. No. 16/096,557 is a national phase application under 35 U.S.C. § 371 of International PCT Application PCT/US2017/029476, filed on Apr. 25, 2017, which claims benefit of and priority to U.S. Provisional Patent Application 62/327,880 filed on Apr. 26, 2016; U.S. patent application Ser. No. 16/096,557 is also a continuation-in-part of U.S. patent application Ser. No. 14/391,122, filed on Oct. 7, 2014, which is a national phase application under 35 U.S.C. § 371 of International PCT Application PCT/US2013/036456, filed on Apr. 12, 2013, which claims benefit of and priority to U.S. Provisional Patent Application 61/624,175, filed on Apr. 13, 2012; International PCT Application PCT/US2021/027418 is also a continuation-in-part of U.S. patent application Ser. No. 16/946,947, filed on Jul. 13, 2020, which is a continuation of U.S. patent application Ser. No. 16/462,916, filed on May 21, 2019; U.S. patent application Ser. No. 16/462,916 is a national phase application under 35 U.S.C. § 371 of International PCT Application PCT/US2017/062634, filed on Nov. 20, 2017, which claims benefit of and priority to U.S. Provisional Patent Application 62/426,126, filed on Nov. 23, 2016 and to U.S. Provisional Patent Application 62/551,649, filed on Aug. 29, 2017; U.S. patent application Ser. No. 16/462,916 is also a continuation of U.S. patent application Ser. No. 16/082,793, filed on Sep. 6, 2018, which is a national phase application under 35 U.S.C. § 371 of International PCT Application PCT/US2017/020805, filed on Mar. 3, 2017, which claims benefit of and priority to U.S. Provisional Patent Application 62/305,892, filed on Mar. 9, 2016, and to U.S. Provisional Patent Application 62/370,174, filed on Aug. 2, 2016; U.S. patent application Ser. No. 16/462,916 is also a continuation-in-part of U.S. patent application Ser. No. 14/951,410, filed on Nov. 24, 2015, which claims benefit of and priority to U.S. Provisional Patent Application 62/085,179, filed on Nov. 26, 2014, and to U.S. Provisional Patent Application 62/248,181, filed on Oct. 29, 2015; U.S. patent application Ser. No. 14/951,410 is a continuation-in-part of U.S. patent application Ser. No. 14/401,081, filed on Nov. 13, 2014, which is a national phase application under 35 U.S.C. § 371 of International PCT Application PCT/US2013/042765, filed on May 24, 2013, which claims benefit of and priority to U.S. Provisional Patent Application 61/652,021, filed on May 25, 2012; U.S. patent application Ser. No. 14/951,410 is also a continuation-in-part of U.S. patent application Ser. No. 14/468,778, filed on Aug. 26, 2014, which is a continuation of U.S. patent application Ser. No. 13/479,137, filed on May 23, 2012, which is a continuation of U.S. patent application Ser. No. 13/049,750, filed on Mar. 16, 2011; U.S. patent application Ser. No. 13/479,137 is also a continuation-in-part of U.S. patent application Ser. No. 12/971,576, filed on Dec. 17, 2010, which claims benefit of and priority to U.S. Provisional Patent Application 61/289,319, filed on Dec. 22, 2009; International PCT Application PCT/US2021/027418 is also a continuation-in-part of U.S. patent application Ser. No. 16/950,774, filed on Nov. 17, 2020, which is a continuation of U.S. patent application Ser. No. 16/608,157, filed on Oct. 25, 2019, which is a national phase application under 35 U.S.C. § 371 of International PCT Application PCT/US2018/02947, filed on Apr. 25, 2018, which claims benefit of and priority to U.S. Provisional Patent Application 62,490,457, filed on Apr. 26, 2017 and titled “ELECTROCHROMIC WINDOWS WITH TRANSPARENT DISPLAY TECHNOLOGY,” U.S. Provisional Patent Application 62,506,514, filed on May 15, 2017 and titled “ELECTROCHROMIC WINDOWS WITH TRANSPARENT DISPLAY TECHNOLOGY,” U.S. Provisional Patent Application 62,507,704, filed on May 17, 2017 and titled “ELECTROCHROMIC WINDOWS WITH TRANSPARENT DISPLAY TECHNOLOGY,” U.S. Provisional Patent Application 62,523,606, filed on Jun. 22, 2017 and titled “ELECTROCHROMIC WINDOWS WITH TRANSPARENT DISPLAY TECHNOLOGY,” and U.S. Provisional Patent Application 62,607,618, filed on Dec. 19, 2017 and titled “ELECTROCHROMIC WINDOWS WITH TRANSPARENT DISPLAY TECHNOLOGY;” International PCT Application PCT/US2021/027418 is also a continuation-in-part of U.S. patent application Ser. No. 17/083,128, filed on Oct. 28, 2021; International PCT Application PCT/US2021/027418 is also a continuation-in-part of U.S. patent application Ser. No. 17/081,809, filed on Oct. 27, 2020, which is a continuation of U.S. patent application Ser. No. 16/608,159, filed on Oct. 24, 2019, which is a national phase application under 35 U.S.C. § 371 of International PCT Application PCT/US2018/029406, filed on Apr. 25, 2018, claims benefit of and priority to U.S. Provisional Patent Application 62,490,457, U.S. Provisional Patent Application 62,506,514, U.S. Provisional Patent Application 62,507,704, U.S. Provisional Patent Application 62,523,606, and U.S. Provisional Patent Application 62,607,618; International PCT Application PCT/US2021/027418 is also a continuation-in-part of International PCT Application PCT/US2020/053641, filed on Sep. 30, 2020, which claims benefit of and priority to U.S. Provisional Patent Application 62/911,271, filed on Oct. 5, 2019, U.S. Provisional Patent Application 62/952,207, filed on Dec. 20, 2019, U.S. Provisional Patent Application 62/975,706, filed on Feb. 12, 2020, and to U.S. Provisional Patent Application 63/085,254, filed on Sep. 30, 2020; International PCT Application PCT/US2020/053641 is a continuation-in-part of U.S. patent application Ser. No. 16/608,157, filed on Oct. 24, 2019; all of these applications are incorporated by reference herein in their entireties and for all purposes.

### FIELD

Certain aspects pertain generally to methods, apparatus, and systems for controlling one or more building systems.

### BACKGROUND

Information about building wellness may be of upmost importance in a world struggling to manage and recover from the global pandemic caused by COVID-19. For example, such information may be critical for tenants to (i) ensure a healthy environment for employees, so they are productive and feel safe in the office (ii) respond quickly to crises with staffing processes and policies for protecting employee safety and ensuring business continuity and (iii) understand building and space performance for real estate leases. Building wellness information may also be important to employees to (i) manage personal safety when planning trips to and from the office, and (ii) enjoy a sense of comfort and safety in knowing that the environmental and wellness conditions of the office are being rigorously monitored.

### SUMMARY

Certain embodiments pertain to systems having at least one controller for controlling one or more building systems based on a plurality of composite indices of one or more enclosures, the at least one controller configured to determine the plurality of composite indices based at least in part on sensor data from a plurality of sensors.

Certain embodiments pertain to methods of controlling one or more building systems. The methods determine a plurality of composite indices of one or more enclosures based at least in part on sensor data from a plurality of sensors and control the one or more building systems based on the determined plurality of composite indices.

Certain embodiments pertain to non-transitory computer program product comprising a computer readable memory storing computer executable instructions for controlling the one or more building systems, including one or more tintable windows. The computer executable instructions, when read by one or more processors operatively coupled to the one or more building systems, cause the one or more processors to execute operations of a method that determines a plurality of composite indices of one or more enclosures based at least in part on sensor data from a plurality of sensors and controls the one or more building systems based on the determined plurality of composite indices.

Certain embodiments pertain to systems having a plurality of sensors, at least one controller, and a user interface. The plurality of sensors is at a building and is configured to generate sensor data associated with an environment in the building. The at least one controller is configured to receive the sensor data, and determine, based at least in part on the sensor data, a first composite index and a second composite index of a space of the building. The user interface is configured to present information associated with the first composite index and the second composite index.

Certain embodiments pertain to systems having at least one controller configured to receive sensor data taken by a plurality of sensors and determine, based at least on part on the sensor data, a plurality of composite indices of one or more enclosures.

Certain embodiments pertain to methods that obtain sensor data taken by a plurality of sensors and determine a plurality of composite indices of one or more enclosures based at least in part on sensor data from the plurality of sensors.

Certain embodiments pertain to non-transitory computer program product comprising a computer readable memory storing computer executable instructions. The computer executable instructions, when read by one or more processors, cause the one or more processors to execute operations of a method that obtains sensor data taken by a plurality of sensors and determines a plurality of composite indices of one or more enclosures based at least in part on sensor data from the plurality of sensors.

These and other features are described in more detail below with reference to the associated drawings.

### BRIEF DESCRIPTION OF THE DRAWINGS

FIG. 1 depicts a schematic drawing of a cross-section of an electrochromic device, in accordance with some embodiments.

FIG. 2 depicts a schematic drawing of a cross-section of an electrochromic device in a bleached state, in accordance with some embodiments.

FIG. 3 depicts a schematic drawing of a cross-section of an electrochromic device in a colored state, in accordance with some embodiments.

FIG. 4 depicts a drawing of a cross-section of an insulated glass unit (IGU), in accordance with some embodiments.

FIG. 5 depicts a schematic drawing of an example of an arrangement of sensors including one or more ensembles of sensors in one or more enclosures, in accordance with some embodiments.

FIG. 6 depicts a schematic diagram of an example of an arrangement of sensor ensembles in an enclosure, in accordance with some embodiments.

FIG. 7A depicts examples of various time windows that include time spans for sensor data collection, in accordance with some embodiments.

FIG. 7B depicts examples of various time windows that include time spans for sensor data collection, in accordance with some embodiments.

FIG. 7C depicts examples of various time windows that include time spans for sensor data collection, in accordance with some embodiments.

FIG. 7D depicts examples of various time windows that include time spans for sensor data collection, in accordance with some embodiments.

FIG. 7E depicts examples of various time windows that include time spans for sensor data collection, in accordance with some embodiments.

FIG. 8 depicts a graph of sensor readings of carbon dioxide (CO₂) concentration level over time in an enclosure, in accordance with some embodiments.

FIG. 9 shows a contour map of a top view of an example an office environment of an enclosure depicting various levels of CO₂ concentrations, in accordance with some embodiments.

FIG. 10 depicts a schematic diagram of an example of a BMS for managing one or more building systems of a building and a control system, in accordance with some embodiments.

FIG. 11 depicts a ventilation system for ventilating an enclosure (e.g., room) inside a building, in accordance with some embodiments.

FIG. 12 depicts a control system for controlling ventilation and other parameters in an enclosure, in accordance with some embodiments.

FIG. 13 depicts a schematic diagram of an example of a system having sensors of a sensor ensemble organized into a sensor module, in accordance with some embodiments.

FIG. 14 depicts a schematic diagram of an example of a control system architecture with a master controller that controls floor controllers, that in turn control local controllers, in accordance with some embodiments.

FIG. 15 depicts a schematic diagram of an example of a computing system and a network, in accordance with some embodiments.

FIG. 16 depicts a representation of an example of a digital twin, in accordance with some embodiments.

FIG. 17 depicts a schematic diagram of an example of a control system that may employ a digital twin in managing and controlling interactive network devices such as building system(s), in accordance with some embodiments.

FIG. 18 depicts a block diagram of an exemplary architecture for managing one or more building systems, in accordance with some embodiments.

FIG. 19 depicts a block diagram of an exemplary computer system for managing one or more building systems, in accordance with some embodiments.

FIG. 20A depicts a graph with an example of a scoring plot for determining scores of a first contributing metric, in accordance with some embodiments.

FIG. 20B depicts a table of values of the first contributing metric of FIG. 20A associated with metric scores derived from the scoring plot in FIG. 20A, in accordance with some embodiments.

FIG. 21A depicts a graph with an example of a scoring plot for determining scores of a second contributing metric, in accordance with some embodiments.

FIG. 21B depicts a table of values of the second contributing metric associated with metric scores derived from the scoring plot in FIG. 21A, in accordance with some embodiments.

FIG. 22 is an example of a dashboard display with real-time data for a facility such as a building, in accordance with some embodiments.

FIG. 23 is an example of a dashboard display with real-time data for a space of a facility, in accordance with some embodiments.

FIG. 24 is an example of a dashboard display with real-time data for carbon dioxide (CO₂) level metric for spaces of a tenant in a facility, in accordance with some embodiments.

FIG. 25 is a flowchart depicting an example of a method of determining a plurality of composite indices for one or more enclosure and/or controlling one or more building systems based on a plurality of composite indices, in accordance with various embodiments.

FIG. 26 is a flowchart depicting operations of a control system that is operatively coupled to one or more devices in an enclosure, in accordance with various embodiments.

FIG. 27A is a graph depicting an example of carbon dioxide sensor data values plotted as a function of time, in accordance with various embodiments.

FIG. 27B is a graph depicting an example of noise sensor data values plotted as a function of time, in accordance with various embodiments.

The figures and components therein may not be drawn to scale. Various components of the figures described herein may not be drawn to scale.

### DETAILED DESCRIPTION

Different aspects are described below with reference to the accompanying drawings. The features illustrated in the drawings may not be to scale. In the following description, numerous specific details are set forth in order to provide a thorough understanding of the presented implementations. The disclosed implementations may be practiced without one or more of these specific details. In other instances, well-known operations have not been described in detail to avoid unnecessarily obscuring the disclosed implementations. While the disclosed implementations will be described in conjunction with the specific implementations, it will be understood that it is not intended to limit the disclosed implementations.

Certain techniques disclosed herein relate generally to methods and systems for determining composite indices or an enclosure (e.g., a building or a space in a building) and/or controlling one or more building systems based on the composite indices. Composite indices may provide a multi-modal assessment of how safe, healthy, and operationally efficient a building may be for its occupants and provide actionable insights into ways of improving the composite indices scores. For example, these techniques may determine remediation action(s) that improve the scores and notify (e.g., via a digital twin of a building) occupants or others that can implement the remediation action(s). These techniques may calculate, for example, three composite indices: a safety index, a health index, and a performance index for the enclosure. Each composite index is calculated by combining weighted scores of contributing metrics from, for example, multiple sources such as sensors in a sensor ensemble including environmental sensors and other sources of data such as building access data, surveys, work orders, user input, and/or utility monitoring. In certain cases, these techniques dynamically adjust contributing metrics and their weighting factors based on availability of data.

Numeric ranges are inclusive of the numbers defining the range. It is intended that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The term “tintable window” refers to a window (e.g., an architectural window) comprising one or more optically switchable devices (e.g., electrochromic devices). An example of a tintable window is an electrochromic window having one or more electrochromic devices. In examples involving commissioning of tintable windows, a tintable window is sometimes referred to as an “insulated glass unit” or “IGU.”

The headings provided herein are not intended to limit the disclosure.

Unless defined otherwise herein, all technical and scientific terms used herein have the same meaning as commonly understood by one of ordinary skill in the art. Various scientific dictionaries that include the terms included herein are well known and available to those in the art. Although any methods and materials similar or equivalent to those described herein find use in the practice or testing of the embodiments disclosed herein, some methods and materials are described.

The terms defined immediately below are more fully described by reference to the Specification as a whole. It is to be understood that this disclosure is not limited to the particular methodology, protocols, and reagents described, as these may vary, depending upon the context they are used by those of skill in the art.

As used herein, the singular terms “a,” “an,” and “the” include the plural reference unless the context clearly indicates otherwise.

### I. Building Systems

In order to orient the reader to certain embodiments of systems, apparatus, and methods disclosed herein, a discussion of building systems including, for example, one or more tintable windows (e.g., electrochromic windows) is provided. This initial discussion is provided for context only, and embodiments described herein are not limited to the specific features or processes of this initial discussion. Moreover, it would be understood that a tintable window may include one or more electrochromic devices in some aspects, and in addition or alternatively, include one or more other optically switchable devices in other aspects.

### Electrochromic Devices

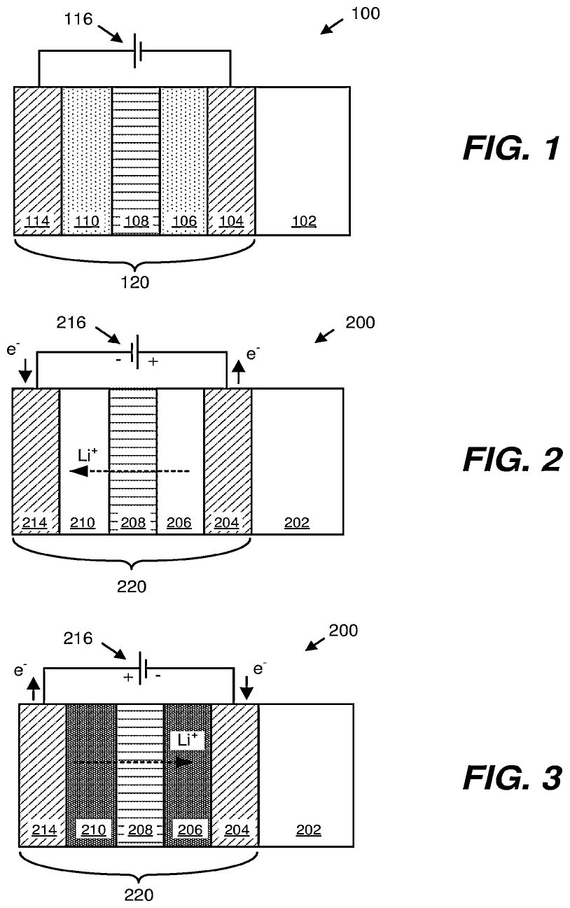

FIG. 1 schematically depicts an electrochromic device 100, in cross-section. Electrochromic device 100 includes a substrate 102, a first conductive layer (CL) 104, an electrochromic layer (EC) 106, an ion conducting layer (IC) 108, a counter electrode layer (CE) 110, and a second conductive layer (CL) 114. Layers 104, 106, 108, 110, and 114 are collectively referred to as an electrochromic stack 120. A voltage source 116 operable to apply an electric potential across electrochromic stack 120 effects the transition of the electrochromic device from, for example, a bleached state to a colored state (depicted). The order of layers can be reversed with respect to the substrate.

Electrochromic devices having distinct layers as described can be fabricated as all solid-state devices and/or all inorganic devices. Such devices and methods of fabricating them are described in more detail in U.S. patent application Ser. No. 12/645,111, entitled “Fabrication of Low-Defectivity Electrochromic Devices,” filed on Dec. 22, 2009, and naming Mark Kozlowski et al. as inventors, and in U.S. patent application Ser. No. 12/645,159, entitled, “Electrochromic Devices,” filed on Dec. 22, 2009 and naming Zhongchun Wang et al. as inventors, both of which are hereby incorporated by reference in their entireties. It should be understood, however, that any one or more of the layers in the stack may contain some amount of organic material. The same can be said for liquids that may be present in one or more layers in small amounts. It should also be understood that solid state material may be deposited or otherwise formed by processes employing liquid components such as certain processes employing sol-gels or chemical vapor deposition.

Additionally, it should be understood that the reference to a transition between a bleached state and colored state is non-limiting and suggests only one example, among many, of an electrochromic transition that may be implemented. Unless otherwise specified herein (including the foregoing discussion), whenever reference is made to a bleached-colored transition (or equivalently a clear-tinted transition), the corresponding device or process encompasses other optical state transitions such as non-reflective-reflective, transparent-opaque, etc. Further, the term “bleached” or “clear” refers to an optically neutral state, for example, uncolored, transparent, or translucent. Still further, unless specified otherwise herein, the “color” or “tint” of an electrochromic transition is not limited to any particular wavelength or range of wavelengths. As understood by those of skill in the art, the choice of appropriate electrochromic and counter electrode materials governs the relevant optical transition.

In embodiments described herein, the electrochromic device reversibly cycles between a bleached/clear state and a colored/tinted state. In some cases, when the device is in a bleached state, a potential is applied to the electrochromic stack 120 such that available ions in the stack reside primarily in the counter electrode 110. When the potential on the electrochromic stack is reversed, the ions are transported across the ion conducting layer 108 to the electrochromic material 106 and cause the material to transition to the colored state. In a similar way, the electrochromic device of embodiments described herein can be reversibly cycled between different tint levels (e.g., bleached state, darkest colored state, and intermediate levels between the bleached state and the darkest colored state).

Referring again to FIG. 1, voltage source 116 may be configured to operate in conjunction with radiant and other environmental sensors. As described herein, voltage source 116 interfaces with a device controller (not shown in this figure). Additionally, voltage source 116 may interface with an energy management system that controls the electrochromic device according to various criteria such as the time of year, time of day, and measured environmental conditions. Such an energy management system, in conjunction with large area electrochromic devices (e.g., an electrochromic window), can dramatically lower the energy consumption of a building.

Any material having suitable optical, electrical, thermal, and mechanical properties may be used as substrate 102. Such substrates include, for example, glass, plastic, and mirror materials. Suitable glasses include either clear or tinted soda lime glass, including soda lime float glass. The glass may be tempered or untempered.

In many cases, the substrate is a glass pane sized for residential window applications. The size of such glass pane can vary widely depending on the specific needs of the residence. In other cases, the substrate is architectural glass. Architectural glass is typically used in commercial buildings, but may also be used in residential buildings, and typically, though not necessarily, separates an indoor environment from an outdoor environment. In certain embodiments, architectural glass is at least 20 inches by 20 inches, and can be much larger, for example, as large as about 80 inches by 120 inches. Architectural glass is typically at least about 2 mm thick, typically between about 3 mm and about 6 mm thick. Of course, electrochromic devices are scalable to substrates smaller or larger than architectural glass. Further, the electrochromic device may be provided on a mirror of any size and shape.

On top of substrate 102 is conductive layer 104. In certain embodiments, one or both of the conductive layers 104 and 114 is inorganic and/or solid. Conductive layers 104 and 114 may be made from a number of different materials, including conductive oxides, thin metallic coatings, conductive metal nitrides, and composite conductors. Typically, conductive layers 104 and 114 are transparent at least in the range of wavelengths where electrochromism is exhibited by the electrochromic layer. Transparent conductive oxides include metal oxides and metal oxides doped with one or more metals. Examples of such metal oxides and doped metal oxides include indium oxide, indium tin oxide, doped indium oxide, tin oxide, doped tin oxide, zinc oxide, aluminum zinc oxide, doped zinc oxide, ruthenium oxide, doped ruthenium oxide and the like. Since oxides are often used for these layers, they are sometimes referred to as “transparent conductive oxide” (TCO) layers. Thin metallic coatings that are substantially transparent may also be used, as well as combinations of TCO's and metallic coatings.

In some embodiments, commercially available substrates such as glass substrates contain a transparent conductive layer coating. Such products may be used for both substrate and conductive layer. Examples of such glasses include conductive layer coated glasses sold under the trademark TEC Glass™ by Pilkington, of Toledo, Ohio and SUNGATE™ 300 and SUNGATE™ 500 by PPG Industries of Pittsburgh, Pennsylvania. TEC Glass™ is a glass coated with a fluorinated tin oxide conductive layer.

In some embodiments of the invention, the same conductive layer is used for both conductive layers (i.e., conductive layers). In some embodiments, different conductive materials are used for each conductive layers. For example, in some embodiments, TEC Glass™ is used for substrate (float glass) and conductive layer (fluorinated tin oxide) and indium tin oxide (ITO) is used for conductive layer. In some embodiments employing TEC Glass™ there is a sodium diffusion barrier between the glass substrate and TEC conductive layer. The function of the conductive layers is to spread an electric potential provided by voltage source 116 over surfaces of the electrochromic stack 120 to interior regions of the stack, with relatively little ohmic potential drop. The electric potential is transferred to the conductive layers though electrical connections to the conductive layers. In some embodiments, bus bars, one in contact with conductive layer 104 and one in contact with conductive layer 114, provide the electric connection between the voltage source 116 and the conductive layers 104 and 114. The conductive layers 104 and 114 may also be connected to the voltage source 116 with other conventional means.

Overlaying conductive layer 104 is electrochromic layer 106. In some embodiments, electrochromic layer 106 is inorganic and/or solid. The electrochromic layer may contain any one or more of a number of different electrochromic materials, including metal oxides. Such metal oxides include tungsten oxide (WO₃), molybdenum oxide (MoO₃), niobium oxide (Nb₂O₅), titanium oxide (TiO₂), copper oxide (CuO), iridium oxide (Ir₂O₃), chromium oxide (Cr₂O₃), manganese oxide (Mn₂O₃), vanadium oxide (V₂O₅), nickel oxide (Ni₂O₃), cobalt oxide (Co₂O₃) and the like. During operation, electrochromic layer 106 transfers ions to and receives ions from counter electrode layer 110 to cause optical transitions.

Generally, the colorization (or change in any optical property—e.g., absorbance, reflectance, and transmittance) of the electrochromic material is caused by reversible ion insertion into the material (e.g., intercalation) and a corresponding injection of a charge balancing electron. Typically some fraction of the ions responsible for the optical transition is irreversibly bound up in the electrochromic material. Some or all of the irreversibly bound ions are used to compensate “blind charge” in the material. In most electrochromic materials, suitable ions include lithium ions (Li+) and hydrogen ions (H+) (that is, protons). In some cases, however, other ions will be suitable. In various embodiments, lithium ions are used to produce the electrochromic phenomena. Intercalation of lithium ions into tungsten oxide (WO_{3-y} (0<y≤˜0.3)) causes the tungsten oxide to change from transparent (bleached state) to blue (colored state).

Referring again to FIG. 1, in electrochromic stack 120, ion conducting layer 108 is sandwiched between electrochromic layer 106 and counter electrode layer 110. In some embodiments, counter electrode layer 110 is inorganic and/or solid. The counter electrode layer may include one or more of a number of different materials that serve as a reservoir of ions when the electrochromic device is in the bleached state. During an electrochromic transition initiated by, for example, application of an appropriate electric potential, the counter electrode layer transfers some or all of the ions it holds to the electrochromic layer, changing the electrochromic layer to the colored state. Concurrently, in the case of NiWO, the counter electrode layer colors with the loss of ions.

In some embodiments, suitable materials for the counter electrode complementary to WO₃ include nickel oxide (NiO), nickel tungsten oxide (NiWO), nickel vanadium oxide, nickel chromium oxide, nickel aluminum oxide, nickel manganese oxide, nickel magnesium oxide, chromium oxide (Cr₂O₃), manganese oxide (MnO₂), and Prussian blue. When charge is removed from a counter electrode 110 made of nickel tungsten oxide (that is, ions are transported from counter electrode 110 to electrochromic layer 106), the counter electrode layer will transition from a transparent state to a colored state.

In the depicted electrochromic device, between electrochromic layer 106 and counter electrode layer 110, there is the ion conducting layer 108. Ion conducting layer 108 serves as a medium through which ions are transported (in the manner of an electrolyte) when the electrochromic device transitions between the bleached state and the colored state. Preferably, ion conducting layer 108 is highly conductive to the relevant ions for the electrochromic and the counter electrode layers, but has sufficiently low electron conductivity that negligible electron transfer takes place during normal operation. A thin ion conducting layer with high ionic conductivity permits fast ion conduction and hence fast switching for high performance electrochromic devices. In certain embodiments, the ion conducting layer 108 is inorganic and/or solid.

Examples of suitable ion conducting layers (for electrochromic devices having a distinct IC layer) include silicates, silicon oxides, tungsten oxides, tantalum oxides, niobium oxides, and borates. These materials may be doped with different dopants, including lithium. Lithium doped silicon oxides include lithium silicon-aluminum-oxide. In some embodiments, the ion conducting layer includes a silicate-based structure. In some embodiments, a silicon-aluminum-oxide (SiAlO) is used for the ion conducting layer 108.

Electrochromic device 100 may include one or more additional layers (not shown), such as one or more passive layers. Passive layers used to improve certain optical properties may be included in electrochromic device 100. Passive layers for providing moisture or scratch resistance may also be included in electrochromic device 100. For example, the conductive layers may be treated with anti-reflective or protective oxide or nitride layers. Other passive layers may serve to hermetically seal electrochromic device 100.

FIG. 2 is a schematic cross-section of an electrochromic device in a bleached state (or transitioning to a bleached state). In accordance with specific embodiments, an electrochromic device 200 includes a tungsten oxide electrochromic layer (EC) 206 and a nickel-tungsten oxide counter electrode layer (CE) 210. Electrochromic device 200 also includes a substrate 202, a conductive layer (CL) 204, an ion conducting layer (IC) 208, and conductive layer (CL) 214.

A power source 216 is configured to apply a potential and/or current to an electrochromic stack 220 through suitable connections (e.g., bus bars) to the conductive layers 204 and 214. In some embodiments, the voltage source is configured to apply a potential of a few volts in order to drive a transition of the device from one optical state to another. The polarity of the potential as shown in FIG. 2 is such that the ions (lithium ions in this example) primarily reside (as indicated by the dashed arrow) in nickel-tungsten oxide counter electrode layer 210.

FIG. 3 is a schematic cross-section of electrochromic device 200 shown in FIG. 2 but in a colored state (or transitioning to a colored state). In FIG. 3, the polarity of voltage source 216 is reversed, so that the electrochromic layer is made more positive to accept additional lithium ions, and thereby transition to the colored state. As indicated by the dashed arrow, lithium ions are transported across ion conducting layer 208 to tungsten oxide electrochromic layer 206. Tungsten oxide electrochromic layer 206 is shown in the colored state. Nickel-tungsten oxide counter electrode 210 is also shown in the colored state. As explained, nickel-tungsten oxide becomes progressively more opaque as it gives up (deintercalates) lithium ions. In this example, there is a synergistic effect where the transition to colored states for both layers 206 and 210 are additive toward reducing the amount of light transmitted through the stack and substrate.

As described above, an electrochromic device may include an electrochromic (EC) layer and a counter electrode (CE) layer separated by an ionically conductive (IC) layer that is highly conductive to ions and highly resistive to electrons. As conventionally understood, the ionically conductive layer therefore prevents shorting between the electrochromic layer and the counter electrode layer. The ionically conductive layer allows the electrochromic and counter electrode layers to hold a charge and thereby maintain their bleached or colored states. In electrochromic devices having distinct layers, the components form a stack which includes the ion conducting layer sandwiched between the electrochromic electrode layer and the counter electrode layer. The boundaries between these three stack components are defined by abrupt changes in composition and/or microstructure. Thus, the devices have three distinct layers with two abrupt interfaces.

In accordance with certain embodiments, the counter electrode and electrochromic layers are formed immediately adjacent one another, sometimes in direct contact, without separately depositing an ionically conducting layer. In some embodiments, electrochromic devices having an interfacial region rather than a distinct IC layer are employed. Such devices, and methods of fabricating them, are described in U.S. Pat. No. 8,300,298 and U.S. patent application Ser. No. 12/772,075 filed on Apr. 30, 2010, and U.S. patent application Ser. Nos. 12/814,277 and 12/814,279, filed on Jun. 11, 2010—each of the three patent applications and patent is entitled “Electrochromic Devices,” each names Zhongchun Wang et al. as inventors, and each is incorporated by reference herein in its entirety.

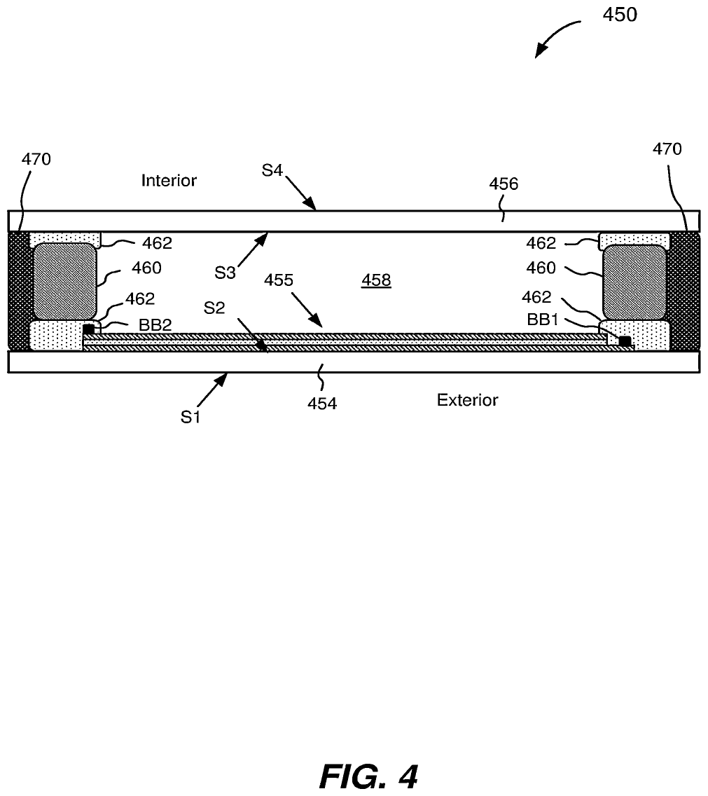

FIG. 4 depicts a cross-sectional view of an example of an electrochromic window including an insulated glass unit (“IGU”) 450, in accordance with some implementations. IGU 450 includes a first pane 454 having a first surface S1 and a second surface S2. In this example, first surface S1 of first pane 454 faces an exterior environment, such as an outdoors or outside environment. IGU 450 also includes a second pane 456 having a third surface S3 and a fourth surface S4. In this example, fourth surface S4 of second pane 456 faces an interior environment, such as an inside environment of a home, building, vehicle, or compartment thereof (e.g., an enclosure therein such as a room). In other examples, the surfaces may face other environments. For example, first surface S1 and fourth surface S4 may face interior environments of a building, for example, when the tintable window functions as a privacy window in a divider wall. Second pane 456 has an electrochromic device coating 455 disposed thereon. Electrochromic device coating 455 includes a first conductive layer, an electrochromic stack, and a second conductive layer. A first bus bar (BB1) is disposed on the first conductive layer and a second bus bar (BB2) is disposed on the second conductive layer.

IGU 500 also includes a 460. Spacer 460 is used to separate the first electrochromic pane (lite) 454 from the second pane (lite) 456. Second pane 456 in IGU 450 is a non-electrochromic lite, however, embodiments disclosed herein are not so limited. For example, second pane 456 may have an electrochromic device thereon and/or one or more coatings such as low-E coatings and the like in other implementations. As another example, second pane 456 can be a laminate of a glass pane laminated to a reinforcing pane with a lamination adhesive such as a resin. Between spacer 460 and first pane 454 is a primary seal material 462. This primary seal material 462 is also between spacer 460 and second pane 456. Around the perimeter of spacer 460 is a secondary seal 470. Secondary seal 470 may be much thicker that depicted. These seals aid in keeping moisture out of an interior volume 458 of IGU 450. They also serve to prevent argon or other gas in the interior volume 458 of IGU 450 from escaping. Bus bar wiring/leads may traverse the seals and/or may pass through spacer 460 to connect to a controller.

In some implementations, the first and the second panes of an IGU (e.g., panes 454 and 456) are transparent or translucent, e.g., at least to light in the visible spectrum. For example, each of the panes can be formed of a glass material. The glass material may include architectural glass, and/or shatter-resistant glass. The glass may comprise a silicon oxide (SOₓ). The glass may comprise a soda-lime glass or float glass. The glass may comprise at least about 75% silica (SiO₂). The glass may comprise oxides such as Na₂O, or CaO. The glass may comprise alkali or alkali-earth oxides. The glass may comprise one or more additives. The first and/or the second panes can include any material having suitable optical, electrical, thermal, and/or mechanical properties. Other materials (e.g., substrates) that can be included in the first and/or the second panes are plastic, semi-plastic and/or thermoplastic materials, for example, poly(methyl methacrylate), polystyrene, polycarbonate, allyl diglycol carbonate, SAN (styrene acrylonitrile copolymer), poly(4-methyl-1-pentene), polyester, and/or polyamide. The first and/or second pane may include mirror material (e.g., silver). In some implementations, the first and/or the second panes can be strengthened. The strengthening may include tempering, heating, and/or chemically strengthening.

A lite of an IGU lite may be a single substrate or a multi-substrate construct. The lite may comprise a laminate, e.g., of two substrates. IGUs (e.g., having double- or triple-pane configurations) can provide a number of advantages over single pane configurations. For example, multi-pane configurations can provide enhanced thermal insulation, noise insulation, environmental protection and/or durability, when compared with single-pane configurations. A multi-pane configuration can provide increased protection for an electrochromic device (ECD). For example, the electrochromic films (e.g., as well as associated layers and conductive interconnects) can be formed on an interior surface of the multi-pane IGU and be protected by an inert gas fill in the interior volume of the IGU. The inert gas fill may provide at least some (heat) insulating function for an IGU. Electrochromic IGUs may have heat blocking capability, e.g., by virtue of a tintable coating that absorbs (and/or reflects) heat and light.

In some embodiments, an insulated glass unit (IGU) includes two (or more) substantially transparent substrates. For example, the IGU may include two panes of glass. At least one substrate of the IGU may include an electrochromic device disposed thereon. The one or more panes of the IGU may have a separator disposed between them. An IGU can be a hermetically sealed construct, e.g., having an interior region that is isolated from the ambient environment. A tintable window may include an IGU and/or a laminate. The tintable window may include one or more electrical leads to supplying power and/or communicating with one of more devices in the tintable window. For example, the electrical leads may operatively couple (e.g. connect) one or more electrochromic devices to a voltage source, switches and the like, and may include a frame that supports the IGU or laminate. An assembly of a tintable window (also referred to herein as a window assembly) may include a window controller, and/or components of a window controller (e.g., a dock).

### Sensors and Ensembles

In some embodiments, an enclosure (e.g., a room) of a structure such as a building includes one or more sensors. The sensor may facilitate controlling the environment of the enclosure such that inhabitants of the enclosure may have an environment that is more comfortable, delightful, beautiful, healthy, productive (e.g., in terms of inhabitant performance), easer to live (e.g., work) in, or any combination thereof. The sensor(s) may be configured as low or high resolution sensors. In some cases, a sensor may provide on/off indications of the occurrence and/or presence of a particular environmental event (e.g., one pixel sensors).

In some embodiments, accuracy and/or resolution of a sensor may be improved via artificial intelligence analysis of its measurements. Examples of artificial intelligence techniques that may be used include: reactive, limited memory, theory of mind, and/or self-aware techniques known to those skilled in the art.

In various implementations, a plurality of sensors may be configured to process, measure, analyze, detect and/or react to one or more of: data, temperature, humidity, sound, force, pressure, electromagnetic waves, position, distance, movement, flow, acceleration, speed, vibration, dust, light, glare, color, gas(es), and/or other aspects (e.g., characteristics) of an environment (e.g., of an enclosure). The gases may include, e.g., volatile organic compounds (VOCs). In addition or alternatively, the gases may include carbon monoxide, carbon dioxide, water vapor (e.g., humidity), oxygen, radon, and/or hydrogen sulfide.

In one embodiment, one or more sensors may be calibrated in a factory setting. For example, a sensor may be optimized to be capable of performing accurate measurements of one or more environmental characteristics present in the factory setting. In some instances, such a factory calibrated sensor may be less optimized when operating in a target environment. For example, a factory setting may comprise a different environment than a target environment. The target environment can be an environment in which the sensor is deployed. The target environment can be an environment in which the sensor is expected and/or destined to operate. The target environment may differ from a factory environment. A factory environment corresponds to a location at which the sensor was assembled and/or built. The target environment may comprise a factory in which the sensor was not assembled and/or built. In some instances, the factory setting may differ from the target environment to the extent that sensor readings captured in the target environment are erroneous (e.g., to a measurable extent). In this context, “erroneous” may refer to sensor readings that deviate from a specified accuracy (e.g., specified by a manufacture of the sensor). In some situations, a factory-calibrated sensor may provide readings that do not meet accuracy specifications (e.g., by a manufacturer) when operated in the target environments.

In one embodiment, one or more shortcomings in sensor operation may be at least partially corrected and/or alleviated by allowing a sensor to be self-calibrated in its target environment (e.g., where the sensor is installed). In some instances, a sensor may be calibrated and/or recalibrated after installation in the target environment. In some instances, a sensor may be calibrated and/or recalibrated after a certain period of operation in the target environment. The target environment may be the location at which the sensor is installed in an enclosure. In comparison to a sensor that is calibrated prior to installation, in a sensor calibrated and/or recalibrated after installation in the target environment may provide measurements having increased accuracy (e.g., that is measurable). In certain embodiments, one or more previously-installed sensors in an enclosure provide readings that are used to calibrated and/or recalibrate a newly-installed sensor in the enclosure. A calibrated and/or localized component may be utilized as a standard for calibrating and/or localizing other components. Such component may be referred to as the “golden component.” The golden component be utilized as a reference component. Such component may be the one most calibrated and/or accurately localized in the facility. The component (e.g., sensor, emitter, or transceiver) may be calibrated and/or localized via a traveler. The traveler may be human or non-human (e.g., robotic). The traveler may be a field service engineer. The traveler may comprise a mobile robot such as a drone, a wheeled robot, or any other maneuverable robot. Examples of components (e.g., devices), control, calibration, and travelers can be found in International Patent Application Serial No. PCT/US21/15378 that is incorporated herein by reference in its entirety.

In some embodiments, a target environment corresponding to a first enclosure differs from a target environment corresponding to a second enclosure. For example, a target environment of an enclosure that corresponds to a cafeteria or to an auditorium may present sensor readings different than a target enclosure that corresponds to a conference room. A sensor may consider the target environment (e.g., one or more characteristics thereof) when performing sensor readings and/or outputting sensor data. For example, during lunchtime a carbon dioxide sensor installed in an occupied cafeteria may provide higher readings than a sensor installed in an empty conference room. In another example, ambient noise sensor located in an occupied cafeteria during lunch may provide higher readings than an ambient noise sensor located in a library.

In some embodiments, a sensor (e.g., occasionally) provides an output signal indicating an erroneous measurement. The sensor may be operatively coupled to at least one controller. The controller(s) may obtain erroneous sensor reading from the sensor. The controller(s) may obtain readings of the same type, at a similar time (e.g., or simultaneously), from one or more other (e.g., nearby) sensors. The one or more other sensors may be disposed at the same environment as the one sensor. The controller(s) may evaluate the erroneous sensor reading in conjunction with one or more readings of the same type made by one or more other sensors of the same type to identify the erroneous sensor reading as an outlier. For example, the controller may evaluate an erroneous temperature sensor reading and one or more readings of temperature made by one or more other temperature sensors. The controller(s) may determine that the sensor reading is erroneous in response to consideration (e.g., including evaluating and/or comparing with) the sensor reading with one or more readings from other sensors in the same environment (e.g., in the same enclosure). Controller(s) may direct the one sensor providing the erroneous reading to undergo recalibration (e.g., by undergoing a recalibration procedure). For example, the controller(s) may transmit one or more values and/or parameters to the sensor(s) providing the erroneous reading. The sensor(s) providing the erroneous reading may utilize the transmitted value and/or parameter to adjust its subsequent sensor reading(s). For example, the sensor(s) providing the erroneous reading may utilize the transmitted value and/or parameter to adjust its baseline for subsequent sensor reading(s). The baseline can be a value, a set of values, or a function.

In some embodiments, a sensor has an operational lifespan. An operational lifespan of a sensor may be related to one or more readings taken by the sensor. Sensor readings from certain sensors may be more valuable and/or varied during certain time periods and may be less valuable and/or varied during other time periods. For example, movement sensor readings may be more varied during the day than during the night. The operational lifespan of the sensor may be extended. Extension of the operational lifespan may be accomplished by permitting the sensor to reduce sampling of environmental parameters at certain time periods (e.g., having the lower beneficial value). Certain sensors may modify (e.g., increase or decrease) a frequency at which sensor readings are sampled. Timing and/or frequency of the sensor operation may depend on the sensor type, location in the (e.g., target) environment, and/or time of day. A sensor type may require constant and/or more frequent operation during the day (e.g., CO₂, volatile organic compounds (VOCs), occupancy, and/or lighting sensor). Volatile organic compounds may be animal and/or human derived. VOCs may comprise a compound related to human produced odor. A sensor may require infrequent operation during at least a portion of the night. A sensor type may require infrequent operation during at least a portion of the day (e.g., temperature and/or pressure sensor). A sensor may be assigned a timing and/or frequency of operation. The assignment may be controlled (e.g., altered) manually and/or automatically (e.g., using at least one controller operatively coupled to the sensor). Operatively coupled may include communicatively coupled, electrically coupled, optically coupled, or any combination thereof. Modification of the timing and/or frequency at which sensor readings are taken may be responsive to detection of an event by a sensor of the same type or of a sensor of a different type. Modification of the timing and/or frequency at which sensor readings may utilize sensor data analysis. The sensor data analysis may utilize artificial intelligence (abbreviated herein as “AI”). The control may be fully automatic or partially automatic. The partially automatic control may allow a user to (i) override a direction of the controller, and/or (ii) indicate any preference (e.g., of the user).

In some embodiments, processing sensor data comprises performing sensor data analysis. The sensor data analysis may comprise at least one rational decision making process, and/or learning. The sensor data analysis may be utilized to adjust the environment, e.g., by adjusting one or more components that affect the environment of the enclosure. The data analysis may be performed by a machine based system (e.g., a circuitry). The circuitry may be of a processor. The sensor data analysis may utilize artificial intelligence. The sensor data analysis may rely on one or more models (e.g., mathematical models). In some embodiments, the sensor data analysis comprises linear regression, least squares fit, Gaussian process regression, kernel regression, nonparametric multiplicative regression (NPMR), regression trees, local regression, semiparametric regression, isotonic regression, multivariate adaptive regression splines (MARS), logistic regression, robust regression, polynomial regression, stepwise regression, ridge regression, lasso regression, elasticnet regression, principal component analysis (PCA), singular value decomposition, fuzzy measure theory, Borel measure, Han measure, risk-neutral measure, Lebesgue measure, group method of data handling (GMDH), Naive Bayes classifiers, k-nearest neighbors algorithm (k-NN), support vector machines (SVMs), neural networks, support vector machines, classification and regression trees (CART), random forest, gradient boosting, or generalized linear model (GLM) technique.

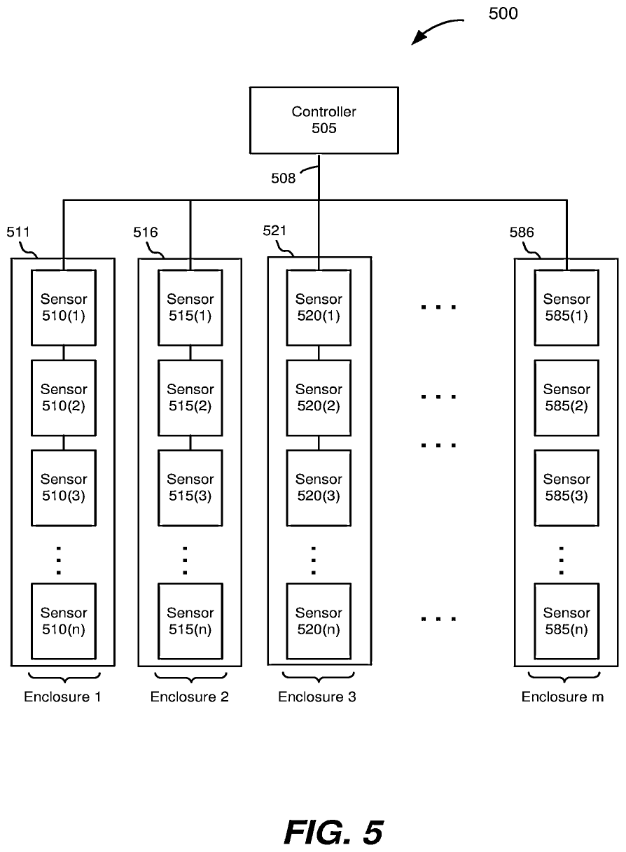

FIG. 5 depicts a schematic diagram 500 of an example of an arrangement of sensors distributed among one or more enclosures. In the example shown in FIG. 5, controller 505 is communicatively linked 508 with sensors 510(1), 510(2), 510(3), . . . 510(n) located in enclosure 1, sensors 515(1), 515(2), 515(3), . . . , 515(n) located in enclosure 2, sensors 520(1), 520(2), 520(3), . . . , 520(n) located in enclosure 3, . . . , and sensors 585(1), 585(2), 585(3), . . . , 585(n) located in enclosure m (where m=1, 2, 3, 4, 5, 6, 7, 8, 9, etc. and n=1, 2, 3, 4, 5, 6, 7, 8, 9, etc.). In other implementations, fewer or more sensors may be located in the enclosures and/or additional or fewer enclosures may be communicatively linked to controller 505. Communicatively linked comprises wired and/or wireless communication.

In some embodiments, an ensemble of sensors may refer to a collection of diverse sensors. In some cases, a sensor ensemble includes at least two sensors of differing types. In other embodiments, a sensor ensemble may include at least two sensors of the same type.

FIG. 5 depicts a first ensemble 511 including sensors 510(1), 510(2), 510(3), . . . , 510(n), a second ensemble 516 including sensors 515(1), 515(2), 515(3), . . . , 515(n), a third ensemble 521 including sensors 520(1), 520(2), 520(3), . . . , 520(n), . . . , and an m^{th} ensemble 586 including sensors 585(1), 585(2), 585(3), . . . , 584(n) (where m=1, 2, 3, 4, 5, 6, 7, etc.). In other implementations, fewer or more ensembles may be included. In the illustrated example, the first ensemble 511 may represent an ensemble of diverse sensors where at least two of sensors 510(1), 510(2), 510(3), . . . , 510(n) are of different types.

In some embodiments, at least two of the sensors in an ensemble cooperate to determine environmental parameters, e.g., of an enclosure in which they are disposed. For example, a sensor ensemble may include a carbon dioxide sensor, a carbon monoxide sensor, a volatile organic chemical sensor, an ambient noise sensor, a visible light sensor, a temperature sensor, and/or a humidity sensor. A sensor ensemble may comprise other types of sensors, and claimed subject matter is not limited in this respect. The enclosure may comprise one or more sensors that are not part of an ensemble of sensors. The enclosure may comprise a plurality of ensembles. At least two of the plurality of ensembles may differ in at least one of their sensors. At least two of the plurality of ensembles may have at least one of their sensors that is similar (e.g., of the same type). For example, an ensemble can have two motion sensors and one temperature sensor. For example, an ensemble can have a carbon dioxide sensor and an IR sensor. The ensemble may include one or more devices that are not sensors. The one or more other devices that are not sensors may include sound emitter (e.g., buzzer), and/or electromagnetic radiation emitters (e.g., light emitting diode). In some embodiments, a single sensor (e.g., not in an ensemble) may be disposed adjacent (e.g., immediately adjacent such as contacting) another device that is not a sensor.

In some embodiments, sensors of a sensor ensemble collaborate with one another (e.g., using the control system). The sensors can comprise an array of sensors. The array of sensors can collaborate synergistically (e.g., using the network and/or controller(s)). The controllers may be included in a control system (e.g., as disclosed herein). A sensor of one type may have a correlation with at least one other type of sensor. A situation in an enclosure may affect one or more of different sensors. Sensor readings of the one or more different may be correlated and/or affected by the situation. The correlations may be predetermined. The correlations may be determined over a period of time (e.g., using a learning process). The period of time may be predetermined. The period of time may have a cutoff value. The cutoff value may consider an error threshold (e.g., percentage value) between a predictive sensor data and a measured sensor data, e.g., in similar situation(s). The time may be ongoing. The correlation may be derived from a learning set (also referred to herein as “training set”). The learning set may comprise, and/or may be derived from, real time observations in the enclosure. The observations may include data collection (e.g., from sensor(s)). The learning set may comprise sensor(s) data from a similar enclosure. The learning set may comprise third party data set (e.g., of sensor(s) data). The learning set may derive from simulation, e.g., of one or more environmental conditions affecting the enclosure. The learning set may compose detected (e.g., historic) signal data to which one or more types of noise were added. The correlation may utilize historic data, third party data, and/or real time (e.g., sensor) data. The correlation between two sensor types may be assigned a value. The value may be a relative value (e.g., strong correlation, medium correlation, or weak correlation). The learning set that is not derived from real-time measurements, may serve as a benchmark (e.g., baseline) to initiate operations of the sensors and/or various components that affect the environment (e.g., HVAC system, and/or tinting windows). Real time sensor data may supplement the learning set, e.g., on
an ongoing basis or for a defined time period. The (e.g., supplemented) learning set may increase in size during deployment of the sensors in the environment. The initial learning set may increase in size, e.g., with inclusion of additional (i) real time measurements, (ii) sensor data from other (e.g., similar) enclosures, (iii) third party data, (iv) other and/or updated simulation.

In some embodiments, data from sensors may be correlated. Once a correlation between two or more sensor types is established, a deviation from the correlation (e.g., from the correlation value) may indicate an irregular situation and/or malfunction of a sensor of the correlating sensors. The malfunction may include a slippage of a calibration. The malfunction may indicate a requirement for re-calibration of the sensor. A malfunction may comprise complete failure of the sensor. In an example, a movement sensor may collaborate with a carbon dioxide sensor. In an example, responsive to a movement sensor detecting movement of one or more individuals in an enclosure, a carbon dioxide sensor may be activated to begin taking carbon dioxide measurements. An increase in movement in an enclosure, may be correlated with increased levels of carbon dioxide. In another example, a motion sensor detecting individuals in an enclosure may be correlated with an increase in noise detected by a noise sensor in the enclosure. In some embodiments, detection by a first type of sensor that is not accompanied by detection by a second type of sensor may result in a sensor posting an error message. For example, if a motion sensor detects numerous individuals in an enclosure, without an increase in carbon dioxide and/or noise, the carbon dioxide sensor and/or the noise sensor may be identified as having failed or as having an erroneous output. An error message may be posted. A first plurality of different correlating sensors in a first ensemble may include one sensor of a first type, and a second plurality of sensors of different types. If the second plurality of sensors indicate a correlation, and the one sensor indicates a reading different from the correlation, there is an increased likelihood that the one sensor malfunctions. If the first plurality of sensors in the first ensemble detect a first correlation, and a third plurality of correlating sensors in a second ensemble detect a second correlation different from the first correlation, there is an increased likelihood that the situation to which the first ensemble of sensors is exposed to is different from the situation to which the third ensemble of sensors are exposed to.

Sensors of a sensor ensemble may collaborate with one another. The collaboration may comprise considering sensor data of another sensor (e.g., of a different type) in the ensemble. The collaboration may comprise trends projected by the other sensor (e.g., type) in the ensemble. The collaboration may comprise trends projected by data relating to another sensor (e.g., type) in the ensemble. The other sensor data can be derived from the other sensor in the ensemble, from sensors of the same type in other ensembles, or from data of the type collected by the other sensor in the ensemble, which data does not derive from the other sensor. For example, a first ensemble may include a pressure sensor and a temperature sensor. The collaboration between the pressure sensor and the temperature sensor may comprise considering pressure sensor data while analyzing and/or projecting temperature data of the temperature sensor in the first ensemble. The pressure data may be (i) of a pressure sensor in the first ensemble, (ii) of pressure sensor(s) in one or more other ensembles, (iii) pressure data of other sensor(s) and/or (iv) pressure data of a third party.

In some embodiments, sensor ensembles, are distributed throughout an enclosure. Sensors of a same type may be dispersed in an enclosure, e.g., to allow measurement of environmental parameters at various locations of an enclosure. Sensors of the same type may measure a gradient along one or more dimensions of an enclosure. A gradient may include a temperature gradient, an ambient noise gradient, or any other variation (e.g., increase or decrease) in a measured parameter as a function of location from a point. A gradient may be utilized in determining that a sensor is providing erroneous measurement (e.g., the sensor has a failure).

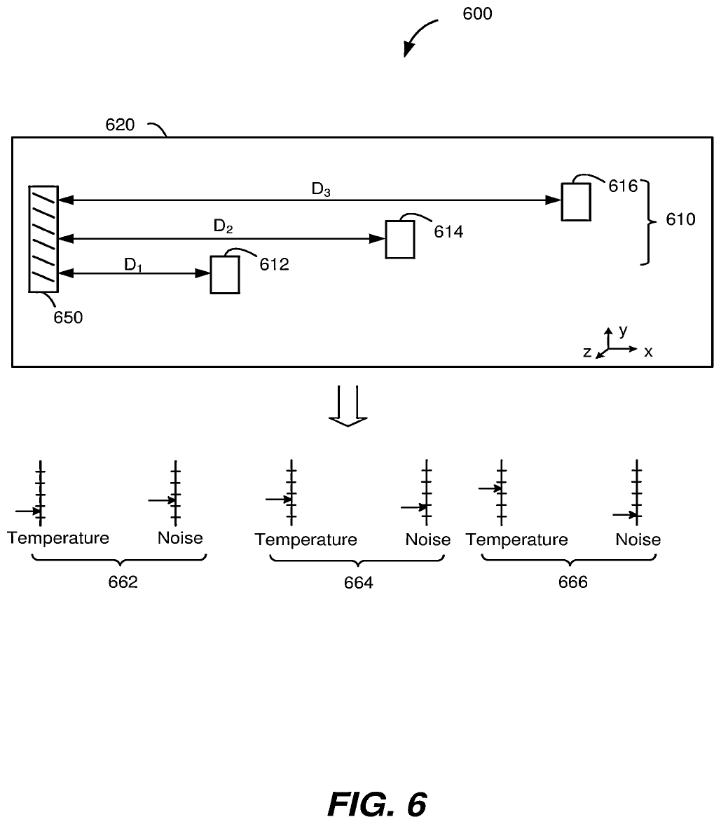

FIG. 6 depicts a schematic diagram 600 of an example of an arrangement of sensor ensembles 610 in an enclosure 620. In the illustrated example, a first ensemble 612 is positioned at a distance D₁ from a vent 650, a second sensor ensemble 614 is positioned at a distance D₂ from vent 650, and a third sensor ensemble 616 is positioned at a distance D₃ from vent 650. Vent 650 may correspond to an air conditioning vent, which represents a relatively constant source of cooling atmosphere and a relatively constant source of white noise. Thus, in this example, temperature and noise measurements may be made by first sensor ensemble 612. Temperature and noise measurements made by sensor 612 are shown by output reading profile 662. Output reading profile 612 indicates a relatively low temperature and a significant amount of noise. Temperature and noise measurements made by second sensor ensemble 614 are shown by output reading profile 664. Output reading profile 664 indicates a somewhat higher temperature, and a somewhat reduced noise level. Temperature and noise measurements made by third sensor ensemble 616 are shown by output reading profile 666. Output reading profile 666 indicates a temperature somewhat higher than the temperature measured by sensor ensembles 614 and 612. Noise measured by third sensor ensemble 616 indicates a lower level than noise measured by sensor ensembles 612 and 614. In an example, if a temperature measured by third sensor ensemble 616 indicates a lower temperature than a temperature measured by first sensor ensemble 612, one or more processors and/or controllers may identify third sensor ensemble 616 sensor as providing erroneous data.

In another example of a temperature gradient, a temperature sensor installed near a window may measure increased temperature fluctuations with respect to temperature fluctuations measured by a temperature sensor installed at a location opposite the window. A sensor installed near a midpoint between the window and the location opposite the window may measure temperature fluctuations in between those measured near a window with respect to those measured at the location opposite the window. In an example, an ambient noise sensor installed near an air conditioner (or near a heating vent) may measure greater ambient noise than an ambient noise sensor installed away from the air conditioning or heating vent.

In some embodiments, a sensor of a first type cooperates with a sensor of a second type. In an example, an infrared radiation sensor may cooperate with a temperature sensor. Cooperation among sensor types may comprise establishing a correlation (e.g., negative or positive) among readings from sensors of the same type or of differing types. For example, an infrared radiation sensor measuring an increase in infrared energy may be accompanied by (e.g., positively correlated to) an increase in measured temperature. A decrease in measured infrared radiation may be accompanied by a decrease in measured temperature. In an example, an infrared radiation sensor measuring an increase in infrared energy that is not accompanied by a measurable increase in temperature, may indicate failure or degradation in operation of a temperature sensor.

In some embodiments, one or more sensors are included in an enclosure. For example, an enclosure may include at least 1, 2, 4, 5, 8, 10, 20, 50, or 500 sensors. The enclosure may include a number of sensors in a range between any of the aforementioned values (e.g., from about 1 to about 1000, from about 1 to about 500, or from about 500 to about 1000). The sensor may be of any type. For example, the sensor may be configured (e.g., and/or designed) to measure concentration of a gas (e.g., carbon monoxide, carbon dioxide, hydrogen sulfide, volatile organic chemicals, or radon). For example, the sensor may be configured (e.g., and/or designed) to measure ambient noise. For example, the sensor may be configured (e.g., and/or designed) to measure electromagnetic radiation (e.g., RF, microwave, infrared, visible light, and/or ultraviolet radiation). For example, the sensor may be configured (e.g., and/or designed) to measure security-related parameters, such as (e.g., glass) breakage and/or unauthorized presence of personnel in a restricted area. Sensors may cooperate with one or more (e.g., active) devices, such as a radar or lidar. The devices may operate to detect physical size of an enclosure, personnel present in an enclosure, stationary objects in an enclosure and/or moving objects in an enclosure.

In some embodiments, the sensor is operatively coupled to at least one controller. The coupling may comprise a communication link. A communications link (e.g., 508 in FIG. 5) may comprise any suitable communications media (e.g., wired and/or wireless). The communication link may comprise a wire, such as one or more conductors arranged in a twisted-pair, a coaxial cable, and/or optical fibers. A communications link may comprise a wireless communication link, such as Wi-Fi, Bluetooth, ZigBee, cellular, or optical. One or more segments of the communications link may comprise a conductive (e.g., wired) media, while one or more other segments of a communications link may comprise a wireless link.

In some embodiments, the enclosure is a facility (e.g., building). The enclosure may comprise a wall, a door, or a window. In some embodiments, at least two enclosures of a plurality of enclosures are disposed in the facility. In some embodiments, at least two enclosures of a plurality of enclosures are disposed different facilities. The different facilities may be a campus (e.g. and belong to the same entity). At least two of the plurality of enclosures may reside in the same floor of the facility. At least two of the plurality of enclosures may reside in different floors of the facility. Enclosures as shown in FIG. 5, such as enclosures 1, 2, 3, . . . , m, may correspond to enclosures located on the same floor of a building, or may correspond to enclosures located on different floors of the building. Enclosures of FIG. 4 may be located in different buildings of a multi-building campus. Enclosures of FIG. 4 may be located in different campuses of a multi-campus neighborhood.

In some embodiments, following installation of a first sensor, a sensor performs self-calibration to establish an operating baseline. Performance of a self-calibration operation may be initiated by an individual sensor, a nearby second sensor, or by one or more controllers. For example, upon and/or following installation, a sensor deployed in an enclosure may perform a self-calibration procedure. A baseline may correspond to a lower threshold from which collected sensor readings may be expected to comprise values higher than the lower threshold. A baseline may correspond to an upper threshold, from which collected sensor readings may be expected to comprise values lower than the upper threshold. A self-calibration procedure may proceed beginning with sensor searching for a time window during which fluctuations or perturbations of a relevant parameter are nominal. In some embodiments, the time window is sufficient to collect sensed data (e.g., sensor readings) that allow separation and/or identification of signal and noise form the sensed data. The time window may be predetermined. The time window may be non-defined. The time window may be kept open (e.g., persist) until a calibration value is obtained.

In some embodiments, a sensor may search for an optimal time to measure a baseline (e.g., in a time window). The optimal time (e.g., in the time window) may be a time span during which (i) the measured signal is most stable and/or (ii) the signal to noise ratio is highest. The measured signal may contain some level of noise. A complete absence of noise may indicate malfunction of the sensor or inadequacy for the environment. The sensed signal (e.g., sensor data) may comprise a time stamp of the measurement of the data. The sensor may be assigned a time window during which it may sense the environment. The time window may be predetermined (e.g., using third party information and/or historical data concerning the property measured by the sensor). The signal may be analyzed during that time window, and an optimal time span may be found in the time window, in which time span the measured signal ism most stable and/or the signal no noise ratio is highest. The time span may be equal to, or shorter than, the time window. The time span may occur during the entire, or during part of the time window.

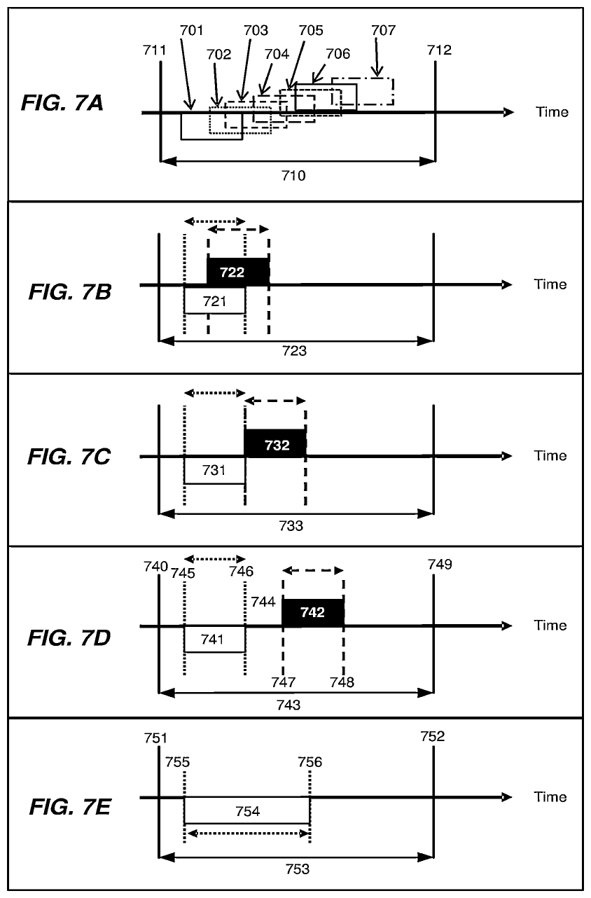

FIG. 7E shows an example of a time window 753 that is indicated having a start time 751 and an end time 752. In the time window 753, a time span 754 is indicated, having a start time 755 and an end time 756. The sensor may sense a property which it is configured to sense (e.g., VOC level) during the time window 753 for the purpose of finding a time span during which an optimal sensed data (e.g., optimal sensed data set) is collected, which optimal data (e.g., data set) has the highest signal to noise ratio, and/or indicates collection of a stable signal. The optimal sensed data may have a (e.g., low) level of noise (e.g., to negate a malfunctioning sensor). For example, a time window may be 12 hours between 5 PM and 5 AM. During that time span, sensed VOC data is collected. The collected sensed data set may be analyzed (e.g., using a processor) to find a time span during the 12h, in which there is a minimal noise level (e.g., indicating that the sensor is functioning) and (i) a highest signal to noise ratio (e.g., the signal is distinguishable) and/or (ii) the signal is most stable (e.g., has a low variability). This time may be of a 1 h duration between 4 AM and 5 AM. In this example, the time window is 12 h between 5 PM and 5 AM, and the time span is 1 h between 4 AM and 5 AM.

In some embodiments, finding the optimal data (e.g., set) to be used for calibration comprises comparing sensor data collected during time spans (e.g., in the time window). In the time window, the sensor may sense the environment during several time spans of (e.g., substantially) equal duration. A plurality of time spans may fit in the time window. The time spans may overlap, or not overlap. The time spans may contract each other. Data collected by the sensors in the various time spans may be compared. The time span having the highest signal to noise and/or having the most stable signal, may be selected as determining the baseline signal. For example, the time window may include a first time span and a second time span. The first time span (e.g., having a first duration, or a first time length) may be shorter than the time windows. The second time span (e.g., having a second duration) may be shorter than the time windows. In some embodiments, evaluating the sensed data (e.g., to find the optimal sensed data used for calibration) comprises comparing a first sensed data set sensed (e.g., and collected) during the first time span, with a second sensed data set sensed (e.g., and collected) during the second time span. The length of the first time span may be different from the length of the second time span. The length of the first time span may be equal (or substantially equal) to the length of the second time span. The first time span may have a start time and/or end time, different than the second time span. The start time and/or end time of the first time span and of the second time span may be in the time window. The start time of the first time span and/or of the second time span, may be equal to the start time of the time window. The end time of the first time span and/or of the second time span, may be equal to the end time of the time window.

FIG. 7D shows an example of a time window 743 having a start time 740 and an end time 749, a first time window 741 having a start time 745 and an end time 746, and a second time window 742 having a start time 747 and an end time 758. In the example shown in FIG. 7D, start times 745 and 747 are in the time window 743, and end times 746 and 748 are in the time window 743.

FIGS. 7A-7D show examples of various time windows that include time spans. FIG. 7A depicts a time lapse diagram in which a time window 710 is indicated having a start time 711 and an end time 712. In the time window 710, various time spans 701-707 are indicated, which time spans overlap each other. The sensor may sense a property which it is configured to sense (e.g., humidity, temperature, or CO₂ level) during at least two of the time spans (e.g., of 701-707), e.g., for the purpose of comparing the signal to find a time at which the signal is most stable and/or has a highest signal to noise ratio. For example, the time window (e.g., 710) may be a day, and the time span (e.g., 701) may be 50 minutes. The sensor may measure a property (e.g., CO₂ level) during overlapping periods of 50 minutes (e.g., during the collective time spans 701-707), and the data may later on be divided into distinct (overlapping) 50 minute time spans, e.g., by using the time stamped measurements. The 50 minutes that indicates the stable CO₂ signal (e.g., at night) and/or having the highest signal to noise, may be designated as an optimal time span for measuring a baseline CO₂ signal. The signal measured may be selected as a baseline for the sensor in one instance. Once the optimal time span has been selected, other CO₂ sensors (e.g., in other locations) can utilize this time span for baseline determination. Finding of the optimal time for baseline determination can speed up the calibration process. Once the optimal time has been found, other sensors may be programmed to measure signal at the optimal time span to record their signal, which may be used for baseline calibration. FIG. 7B depicts a time lapse diagram in which a time window 723 is indicated, during which two time spans 721 and 722 are indicated, which time spans overlap each other. FIG. 7C depicts a time lapse diagram in which a time window 733 is indicated, during which two time spans 731 and 732 are indicated, which time spans contact each other, that is, ending of the first time span 731 is the beginning of the second time span 732. FIG. 7D depicts a time lapse diagram in which a time window 743 is indicated, during which two time spans 741 and 742 are indicated, which time spans are separate by a time gap 744.

In an example, for a carbon dioxide sensor, a relevant parameter may correspond to carbon dioxide concentration. In an example, a carbon dioxide sensor may determine that a time window during which fluctuations in carbon dioxide concentration could be minimal corresponds to a two-hour period, e.g., between 5:00 AM and 7:00 AM. Self-calibration may initiate at 5:00 AM and continue while searching for a duration within these two hours during which measurements are stable (e.g., minimally fluctuating). In some embodiments, the duration is sufficiently long to allow separation between signal and noise. In an example, data from a carbon dioxide sensor may facilitate determination that a 5-minute duration (e.g., between 5:25 AM and 5:30 AM) within a time window between 5:00 AM and 7:00 AM forms an optimal time period to collect a lower baseline. The determination can be performed at least in part (e.g., entirely) at the sensor level. The determination can be performed by one or more processors operatively couple to the sensor. During a selected duration, a sensor may collect readings to establish a baseline, which may correspond to a lower threshold.

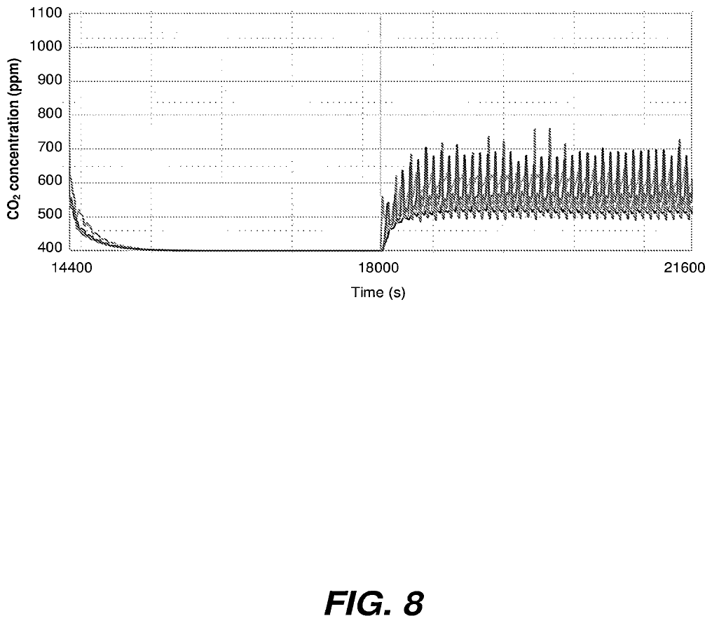

In an example, for gas sensors disposed in a room (e.g., in an office environment), a relevant parameter may correspond to gas (e.g., CO₂) levels, where requested levels are typically in a range of about 1000 ppm or less. In an example, a CO₂ sensor may determine that self-calibration should occur during a time window where CO₂ levels are minimal such as when no occupants are in the vicinity of the sensor (e.g., see CO₂ levels before 18000 seconds in FIG. 8). Time windows during which fluctuations in CO₂ levels are minimal, may correspond to, e.g., a one-hour period during lunch from about 12:00 PM to about 1:00, and during closed business hours.

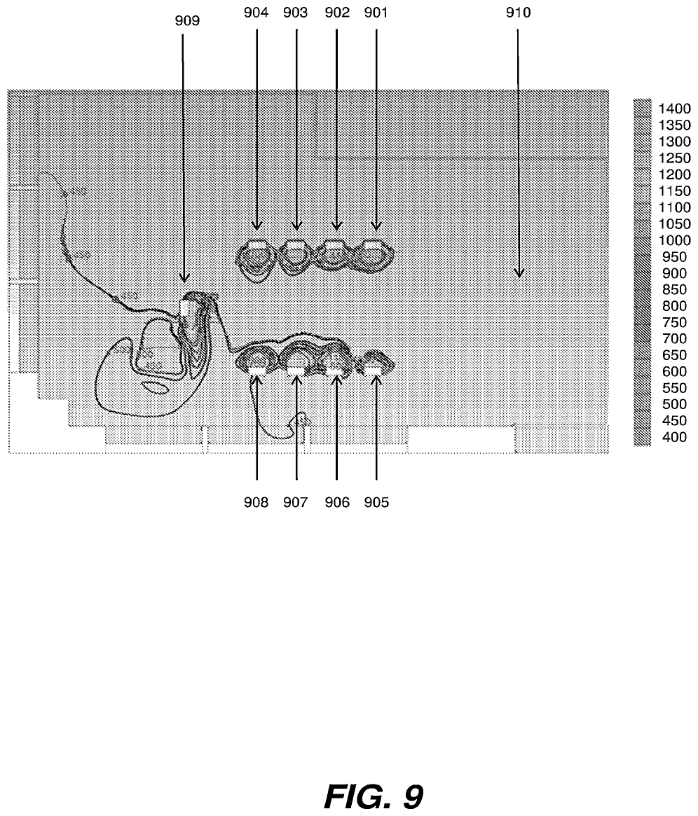

FIG. 9 shows a contour map of a top view of an example an office environment of an enclosure depicting various levels of CO₂ concentrations. The office environment includes a first occupant 901, a second occupant 902, a third occupant 903, a fourth occupant 904, a fifth occupant 905, a sixth occupant 906, a seventh occupant 907, an eighth occupant 908, and a ninth occupant 909. The gas (CO₂) concentrations may be measured by one or more sensors placed at various locations of the enclosure (e.g., office).

In some examples, a source chemical component(s) of the atmosphere material (e.g., VOC) is located using a plurality of sensors in the room. A spatial profile indicating distribution of the chemical(s) in the enclosure may indicate various (e.g., relative or absolute) concentrations of the chemical(s) as a function of space. The profile may be a two or three dimensional profile. The sensors may be disposed in different locations of the room to allow sensing of the chemical(s) in different room locations. Mapping the (e.g., entire) enclosure (e.g., room) may require (i) overlap of sensing regions of the sensors and/or (i) extrapolating distribution of the chemical(s) in the enclosure (e.g., in regions of low or absence of sensor coverage (e.g., sensing regions)). For example, FIG. 9 shows an example of relatively steep and high concentration of carbon dioxide towards where an occupant 905 is present, relative to low concentration in an unoccupied region 910 of the enclosure. This can indicate that in the position of occupant 905 there is a source of carbon dioxide expulsion. Similarly, one can find a location (e.g., source) of chemical removal by finding a (e.g., relatively steep) low concentration of a chemical in the environment. Relative is with respect to the general distribution of the chemical(s) in the enclosure.

### Network

Certain disclosed embodiments provide a network infrastructure in the enclosure (e.g., a facility such as a building). The network infrastructure is available for various purposes such as for providing communication and/or power services. The communication services may comprise high bandwidth (e.g., wireless and/or wired) communications services. The communication services can be to occupants of a facility and/or users outside the facility (e.g., building). The network infrastructure may work in concert with, or as a partial replacement of, the infrastructure of one or more cellular carriers. The network infrastructure can be provided in a facility that includes electrically switchable windows. Examples of components of the network infrastructure include a high speed backhaul. The network infrastructure may include at least one cable, switch, physical antenna, transceivers, sensor, transmitter, receiver, radio, processor and/or controller (that may comprise a processor). The network infrastructure may be operatively coupled to, and/or include, a wireless network. The network infrastructure may comprise wiring. At least a portion of the wiring may be disposed at an envelope of the enclosure (e.g., outer walls of a building). One or more sensors can be deployed (e.g., installed) in an environment as part of installing the network and/or after installing the network.

In various embodiments, a network infrastructure supports a control system. The control system may control one or more building systems including, for example, windows such as tintable (e.g., electrochromic) windows. The control system may comprise one or more controllers operatively coupled (e.g., directly or indirectly) to the one or more windows. The one or more windows may be an optically switchable window, a tintable windows, and/or a smart window. Concepts disclosed herein for electrochromic windows may apply to other types of smart and/or tintable windows (e.g., comprising switchable optical devices) comprising a liquid crystal device, an electrochromic device, suspended particle device (SPD), NanoChromics display (NCD), Organic electroluminescent display (OELD), suspended particle device (SPD), NanoChromics display (NCD), or an Organic electroluminescent display (OELD). The display element may be attached to a part of a transparent body (such as the windows). The tintable window may be disposed in a (non-transitory) facility such as a building, and/or in a transitory vehicle such as a car, buss, train, airplane, helicopter, ship, recreational vehicle, or boat.

### Building Management Systems

In some embodiments, a building management system (BMS) includes a control system installed in a building, that controls (e.g., monitors) one or more building systems such as, e.g., mechanical and/or electrical equipment of an enclosure. The control system may comprise a hierarchy of controllers (e.g., controllers configured for hierarchical communication). The control system may comprise at least one controller that is directed to at least one tintable window. The at least one tintable window may change color, transparency, and/or hue in response to electrical current and/or voltage differential. For example, a control system may control ventilation, lighting, power system, elevator, fire system, and/or security system, of an enclosure of a building. The control systems (e.g., comprising nodes and/or processors) described herein may be suited for integration with a BMS.

A BMS may consist of hardware, including interconnections by communication channels to computer(s) and/or associated software for maintaining conditions in the building, e.g., according to preferences set by at least one user. The user can be an occupant, an owner, a lessor, and/or a building manager. For example, a BMS may be implemented using a local area network, such as Ethernet. The software can include open standards and/or comply with internet protocols, and cellular network protocols (e.g., of at least third generation, fourth generation, or fifth generation cellular network protocol). One example is software from Tridium, Inc. (of Richmond, Va.). One communication protocol commonly used with a BMS is building automation and control networks (BACnet).

In some embodiments, a BMS is disposed in an enclosure such as a facility. The facility can comprise a building such as a multi-story building. The BMS may functions at least to control the environment in the building. The control system and/or BMS may control at least one environmental characteristic of the enclosure. The at least one environmental characteristic may comprise temperature, humidity, fine spray (e.g., aerosol), sound, electromagnetic waves (e.g., light glare, and/or color), gas makeup, gas concentration, gas speed, vibration, volatile compounds (VOCs), debris (e.g., dust), and/or biological matter (e.g., gas borne bacteria and/or virus). The gas(es) may comprise oxygen, nitrogen, carbon dioxide, carbon monoxide, hydrogen sulfide, Nitric oxide (NO) and nitrogen dioxide (NO₂), inert gas, Nobel gas (e.g., radon), cholorophore, ozone, formaldehyde, methane, and/or ethane. For example, a BMS may control temperature, carbon dioxide levels, and/or humidity in an enclosure. Mechanical devices that can be controlled by a BMS and/or control system may comprise lighting, a heater, air conditioner, blower, or vent. To control the enclosure (e.g., building) environment, a BMS and/or control system may turn on and off one or more of the devices it controls, e.g., under defined conditions. A (e.g., core) function of a modern BMS and/or control system may be to maintain a comfortable, healthy, and/or productive environment for the occupant(s) of the enclosure, e.g., while minimizing energy consumption (e.g., while minimizing heating and cooling costs/demand). A modern BMS and/or control system can be used to control (e.g., monitor), and/or to optimize the synergy between various systems, for example, to conserve energy and/or lower enclosure (e.g., facility) operation costs.

In some embodiments, the control system controls at least one environmental characteristic of an enclosure (e.g., atmosphere of the enclosure). The environmental characteristic can be any environmental characteristic disclosed herein. For example, environmental characteristic may be a level of a gas borne and/or gaseous component of the atmosphere. For example, the environmental characteristic may be a level of an atmospheric accumulant. For example, the environmental characteristic may be a level of an atmospheric depletant.

In some embodiments, the control system is operatively (e.g., communicatively) coupled to an ensemble of devices (e.g., comprising one or more sensors and/or emitters). The ensemble facilitates the control of the environment and/or the alert. The control may utilize a control scheme such as feedback control, or any other control scheme delineated herein (e.g., feed forward, closed loop, and/or open loop). The ensemble may comprise at least one sensor configured to sense electromagnetic radiation. The electromagnetic radiation may comprise (humanly) visible, infrared (IR), and/or ultraviolet (UV) radiation. The at least one sensor may comprise an array of sensors. For example, the ensemble may comprise an infrared (IR) sensor array. In addition or alternatively, the ensemble may comprise a sound detector and/or emitter. In addition or alternatively, the ensemble may comprise a microphone. The ensemble may comprise any sensor and/or emitter disclosed herein.

In some embodiments, the ensemble (or a group of ensembles) may be utilized to detect characteristics of enclosure occupant(s). The ensemble may be utilized to detect abnormal bodily characteristic of enclosure occupant(s). The abnormal bodily characteristic may comprise bodily temperature, coughing, sneezing, perspiration (e.g., humidity and/or VOCs expulsion), or CO₂ level. The ensemble(s) may be utilized to locate an absolute and/or relative positioning of enclosure occupant(s). For example, the ensemble(s) may be utilized to measure relative distances between occupants in the enclosure, and/or between occupant(s) and hard and/or dense objects in the enclosure (e.g., fixtures and/or non-fixtures). The hard and/or dense objects may comprise fixtures (e.g., wall, ceiling, floor, window, door, shelf, ceiling light, or wall light) or mobile furniture (e.g., chair, desk, or lamp).

In some examples, one or more sensors in the enclosure may be volatile organic compound (VOC) sensors. A VOC sensor can be specific to a VOC compound (e.g., as disclosed herein), or to a class of compounds (e.g., having similar chemical characteristic). For example, the VOC sensor can be sensitive to aldehydes, esters, thiophenes, alcohols, aromatics (e.g., benzenes and/or toluenes), or olefins. In some cases, one or more VOC sensors may output a total VOC output (also referred to herein as “TVOC”). Sensing can be over a period of time.

In one example, a group of chemical sensors (e.g., sensor array) is sensitive to various chemical compounds (e.g., VOCs) (e.g., having different chemical characteristics). The group of compounds may comprise identified or non-identified chemical compounds. The chemical sensor(s) can output a sensed value of a particular compound, class of compounds, or group of compounds. The sensor output may be of a total (e.g., accumulated) measurements of the class, or group of compounds sensed. The sensor output may be of a total (e.g., accumulated) measurements of multiple sensor outputs of (i) individual compounds, (ii) classes of compounds, or (iii) groups of compounds.

In some embodiments, a local (e.g., window) controller can be integrated with a BMS and/or control system. The local controller can be configured to control one or more devices comprising tintable windows (e.g., comprising an electrochromic window), sensors, emitters, antennas, or any other element communicatively coupled to the network (that is controllable by communication). In one embodiment, the electrochromic windows include at least one all solid state and inorganic electrochromic device. The electrochromic window may include more than one electrochromic device, e.g. where at least two lites (e.g., each lite) are tintable. In one embodiment, the electrochromic windows include (e.g., only) all solid state and inorganic electrochromic devices. In one embodiment, the one or more electrochromic windows include organic electrochromic devices. In one embodiment, the electrochromic windows are multistate electrochromic windows. Examples of tintable windows and their control can be found in U.S. patent application Ser. No. 12/851,514, filed on Aug. 5, 2010, and titled “MULTI-PANE ELECTROCHROMIC WINDOWS” that is incorporated herein by reference in its entirety.

In some embodiments, a plurality of devices may be operatively (e.g., communicatively) coupled to the control system. The plurality of devices may be disposed in a facility (e.g., including a building and/or room). The control system may comprise the hierarchy of controllers. The devices may comprise an emitter, a sensor, or a window (e.g., IGU). The device may be any device as disclosed herein. At least two of the plurality of devices may be of the same type. For example, two or more IGUs may be coupled to the control system. At least two of the plurality of devices may be of different types. For example, a sensor and an emitter may be coupled to the control system. At times the plurality of devices may comprise at least 20, 50, 100, 500, 1000, 2500, 5000, 7500, 10000, 50000, 100000, or 500000 devices. The plurality of devices may be of any number between the aforementioned numbers (e.g., from 20 devices to 500000 devices, from 20 devices to 50 devices, from 50 devices to 500 devices, from 500 devices to 2500 devices, from 1000 devices to 5000 devices, from 5000 devices to 10000 devices, from 10000 devices to 100000 devices, or from 100000 devices to 500000 devices). For example, the number of windows in a floor may be at least 5, 10, 15, 20, 25, 30, 40, or 50. The number of windows in a floor can be any number between the aforementioned numbers (e.g., from 5 to 50, from 5 to 25, or from 25 to 50). At times the devices may be in a multi-story building. At least a portion of the floors of the multi-story building may have devices controlled by the control system (e.g., at least a portion of the floors of the multi-story building may be controlled by the control system). For example, the multi-story building may have at least 2, 8, 10, 25, 50, 80, 100, 120, 140, or 160 floors that are controlled by the control system. The number of floors (e.g., devices therein) controlled by the control system may be any number between the aforementioned numbers (e.g., from 2 to 50, from 25 to 100, or from 80 to 160). The floor may be of an area of at least about 150 m², 250 m², 500 m², 1000 m², 1500 m², or 2000 square meters (m²). The floor may have an area between any of the aforementioned floor area values (e.g., from about 150 m² to about 2000 m², from about 150 m² to about 500 m², from about 250 m² to about 1000 m², or from about 1000 m² to about 2000 m²). The building may comprise an area of at least about 1000 square feet (sqft), 2000 sqft, 5000 sqft, 10000 sqft, 100000 sqft, 150000 sqft, 200000 sqft, or 500000 sqft. The building may comprise an area between any of the above mentioned areas (e.g., from about 1000 sqft to about 5000 sqft, from about 5000 sqft to about 500000 sqft, or from about 1000 sqft to about 500000 sqft). The building may comprise an area of at least about 100 m², 200 m², 500 m², 1000 m², 5000 m², 10000 m², 25000 m², or 50000 m². The building may comprise an area between any of the above mentioned areas (e.g., from about 100 m² to about 1000 m², from about 500 m² to about 25000 m², from about 100 m² to about 50000 m²). The facility may comprise a commercial or a residential building. The commercial building may include tenant(s) and/or owner(s). The residential facility may comprise a multi or a single family building. The residential facility may comprise an apartment complex. The residential facility may comprise a single family home. The residential facility may comprise multifamily homes (e.g., apartments). The residential facility may comprise townhouses. The facility may comprise residential and commercial portions. The facility may comprise at least about 1, 2, 5, 10, 50, 100, 150, 200, 250, 300, 350, 400, 420, 450, 500, or 550 windows (e.g., tintable windows). The components of the facility (e.g., devices such as the windows) may be allocated into zones (e.g., based at least in part on the location, façade, floor, ownership, utilization of the enclosure (e.g., room) in which they are disposed, any other assignment metric, random assignment, or any combination thereof. Allocation of components (e.g., devices such as windows) to the zone may be static or dynamic (e.g., based on a heuristic). There may be at least about 2, 5, 10, 12, 15, 30, 40, or 46 components (e.g., devices such as sensor and/or windows) per zone. The zones may be grouped into groups (e.g., each having a distinguishable name and/or notation). The zones may be clustered (e.g., with each cluster having a distinguishable name and/or notation). The zones, their grouping and/or clustering may form a hierarchy of zones.

In some embodiments, the various components (e.g., IGUs) are grouped into zones of components (e.g., of EC windows). At least one zone (e.g., each of which zones) can include a subset of components (e.g., devices). For example, at least one (e.g., each) zone of components may be controlled by one or more respective floor controllers and one or more respective local controllers (e.g., window controllers) controlled by these floor controllers. In some examples, at least one (e.g., each) zone can be controlled by a single floor controller and two or more local controllers controlled by the single floor controller. For example, a zone can represent a logical grouping of the components (e.g., devices). Each zone may correspond to a set of components (e.g., of the same type) in a specific location or area of the facility that are driven together based at least in part on their location. For example, a facility (e.g., building) may have four faces or sides (a North face, a South face, an East Face, and a West Face) and ten floors. In such a didactic example, each zone may correspond to the set of smart windows (e.g., tintable windows) on a particular floor and on a particular one of the four faces. At least one (e.g., each) zone may correspond to a set of components (e.g., devices) that share one or more physical characteristics (for example, device parameters such as size or age). In some embodiments, a zone of components (e.g., devices) is grouped based at least in part on one or more non-physical characteristics such as, for example, a security designation or a business hierarchy (for example, IGUs bounding managers' offices can be grouped in one or more zones while IGUs bounding non-managers' offices can be grouped in one or more different zones).

In some embodiments, at least one (e.g., each) floor controller is able to address all of the components (e.g., devices) in at least one (e.g., each) of one or more respective zones. The components in the zone may be of the same type or of different types. For example, the master controller can issue a primary tint command to the floor controller that controls a target zone.

In some embodiments, the facility may be divided into one or more zones. The zones may be defined at least in part by a customer, or by the facility manager. The zones may be at least in part automatically defined. For example, the zone of devices (e.g., comprising tintable windows, sensors, or emitters) may associate with (i) a façade of a building they are facing, (ii) a floor they are disposed in, (iii) a building in the facility they are disposed in, (iv) a functionality of the enclosure they are disposed in (e.g., a conference room, a gym, an office, or a cafeteria), (iv) prescribed and/or in fact occupation (e.g., organizational function) to the enclosure they are disposed in, (v) prescribed and/or in fact activity in the enclosure they are disposed in, (vi) tenant, owner, and/or manager of the enclosure of the facility (e.g., for a facility having various tenants, owners, and/or managers), and/or (vii) their geographic location. The zones may be alterable (e.g., using the software app). The status of the zone (e.g., in conjunction to the status of the components (such as devices) therein), may be displayed by the app (e.g., updated in real-time, or substantially in real time). One or more zones may be grouped. For example, all zones in a certain floor may be groped. There may be a zone hierarchy using any of the zone in association with (i) a façade of a building they are facing, (ii) a floor they are disposed in, (iii) a building in the facility they are disposed in, (iv) a functionality of the enclosure they are disposed in (e.g., a conference room, a gym, an office, or a cafeteria), (iv) prescribed and/or in fact occupation (e.g., organizational function) to the enclosure they are disposed in, (v) prescribed and/or in fact activity in the enclosure they are disposed in, (vi) tenant, owner, and/or manager of the enclosure of the facility (e.g., for a facility having various tenants, owners, and/or managers), and/or (vii) their geographic location.

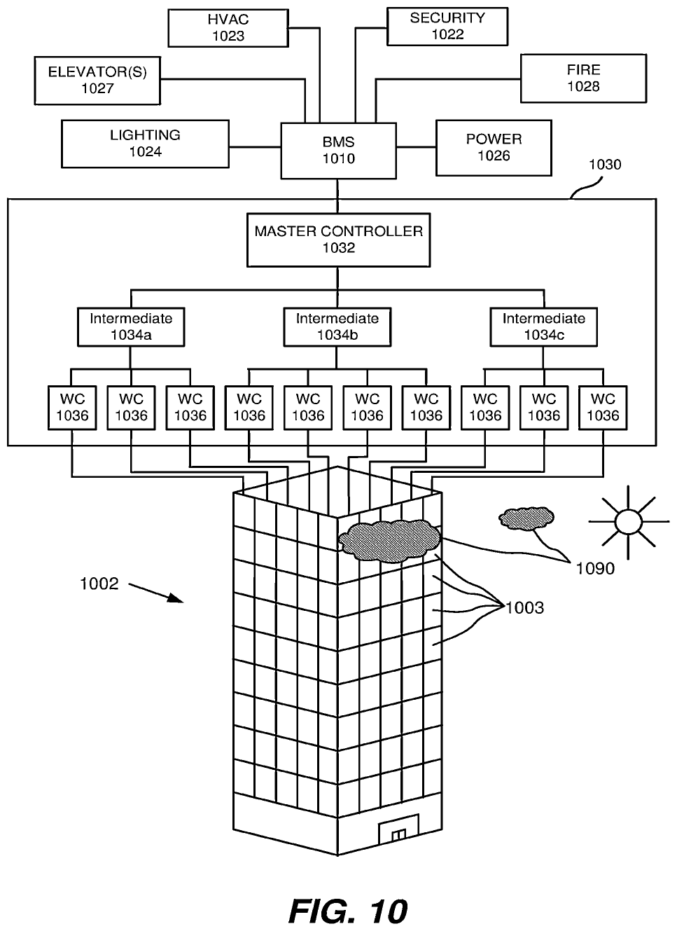

FIG. 10 depicts a schematic diagram of an example of a BMS 1010 for managing one or more building systems of a building 1002 and a control system 1030, according to embodiments. In this example, BMS 1010 manages a number of building systems of building 1002, including security system(s) 1022, a heating ventilation and air conditioning (HVAC) system 1023, a lighting system 1024, power system(s) 1026, elevator(s) system(s) 1027, fire system(s) 1028, and the like. The BMS 1010 may also manage the one or more tintable windows 1002 (e.g., electrochromic windows). Security system(s) 1022 may include, for example, magnetic card access, turnstile, solenoid driven door lock, (e.g., surveillance) camera, (e.g., burglar) alarm, and/or metal detector. BMS 1010 and/or control system 1004 may control at least one fire system and/or fire suppression system (e.g., fire system(s) 1028). The fire system(s) may include one or more fire alarms. The fire suppression system(s) may include a water plumbing control. Lighting system 1024 may include interior lighting, exterior lighting, emergency warning light, emergency exit sign, and/or emergency floor (e.g., egress or ingress) lighting. Power system 1026 may include the main power for the enclosure (e.g., facility), a backup power generator, and/or an uninterrupted power source (UPS). BMS 1010 may manage control system 1030 according to certain aspects. BMS 1010 can be managed by the control system in other aspects. BMS 1010 may be included in control system 1030 in one implementation. At the instant in time shown in FIG. 10, clouds 1090 are passing by building 1002 obstructing sunlight from impinging at least one of the tintable windows 1003.

In FIG. 10, control system 1030 is depicted as a distributed network of controllers. Control system 1030 may have 1, 2, 3, or more hierarchal control levels. In FIG. 10, control system 1030 includes a master controller 1032, intermediate controllers 1034a, 1034b, and 1034c (that can be floor controllers and/or network controllers), and local controllers (e.g., end or leaf controllers such as window controllers) 1036. Other number of intermediate controllers and local controllers may be used in other implementations. Master controller 1032 may or may not be in physical proximity to BMS 1010. At least one floor (e.g., each floor) of building 1002 may have one or more intermediate controllers 1034a, 1034b, and 1034c. At least one device (e.g., window) may have its own local controller 1036. Each local controller 1036 may control any number of devices such as, e.g., at least 1, 2, 3, 4, 5, 6, 7, 8, 9, or 10 devices. Control system 1030 may or may not have intermediate controller(s) in certain implementations.

In the example shown in FIG. 10, at least one (e.g., each) local controller 1036 controls one or more devices. The one or more devices may include a window, a sensor, an emitter, an antenna, a receiver, and/or a transceiver. At least one (e.g., each) local controller 1036 can be disposed in a separate location from the device it controls or be integrated into the device. In the example shown in FIG. 10, ten (10) electrochromic windows of building 1002 are depicted as controlled by master controller 1032. In alternative implementations, there may be fewer or larger number of devices being controlled by master controller 1032.

In some embodiments, the control system may comprise, or be operatively (e.g., communicatively) coupled to, a BMS. By incorporating a (e.g., feedback) control scheme, a BMS and/or control system can provide enhanced: (1) environmental control, (2) energy savings, (3) security, (4) flexibility in control options, (5) improved reliability and usable life of other systems (e.g., coordination of systems may reduce overall operating time of individual systems, leading to less system maintenance), (6) information availability and diagnostics, and/or (7) effective use of, and higher productivity from, staff, and any combination thereof (e.g., because the electrochromic windows can be automatically controlled). In some embodiments, (i) a BMS may not be present, (ii) a BMS may be present but may not communicate with a control system (e.g., with a master controller), or (iii) a BMS may communicate at a high level with the control system (e.g., with a master controller). In certain embodiments, maintenance on the BMS would not interrupt control of the one or more devices (e.g., electrochromic windows) to which the BMS and/or control system is coupled to.

In some embodiments, the BMS and/or the control system controls ventilation within an enclosure. A ventilation system (e.g., as part of an HVAC system) may providing a comfortable environment and good atmospheric (e.g., air) quality. A ventilation system may have significant energy demands. Providing good atmospheric quality to occupants of an enclosure may result in increased wellbeing, comfort, and/or productivity. Such enclosures (e.g., facilities) may be occupied by large number of individuals and/or may be occupied by frequently changing individuals. Such enclosures may include large work environments, health and/or entertainment centers. For example, transportation hubs, sporting hubs, hospitals, exhibition centers, shopping malls, financial centers, movie theaters, museums, and/or cruise ships. The ventilation of an enclosure can exchange the internal environment of the enclosure with the external environment. For example, the ventilation system can bring in outside atmosphere and evacuate inside atmosphere to the environment external to the enclosure (e.g., outside of the facility). The exchange of external and internal atmosphere may adjust one or more components of the internal atmosphere. For example, the exchange of external and internal atmosphere (e.g., by the ventilation system) may reduce any accumulates atmospheric components emitted within the enclosure (e.g., CO₂ from human respiration and VOCs emanating from human breath, saliva, and skin). For example, the exchange of external and internal atmosphere (e.g., by the ventilation system) may alter the levels of oxygen and/or humidity (when their internal and external levels differ). Industry standard(s) may provide recommended ventilation flow rates based at least in part on full occupancy (e.g., number of people), room size, and/or type of facility (e.g., people in an office space generate less CO₂/VOCs than in a gym). Operating a ventilation rate at the recommendation of the industry standard(s) may (e.g., significantly) over-ventilate the enclosure (e.g., when an enclosure (e.g., a room) is occupied at less than maximum occupancy), which may lead to an undesirable energy waste. In addition, a ventilation system may utilize a mixture of outside atmosphere and recirculated inside atmosphere (e.g., at an unknown ratio) for its ventilation. Since the quantity of external atmosphere (e.g., one or more component thereof) may be unknown, the concentration of atmospheric components (e.g., pollutants) may varied (e.g., increased or decreased) to an undesirable level. Further, levels of the atmospheric component(s) may vary as a function of occupancy (e.g., when they are emitted by the occupant), and thus a constant ventilation rate may inadequately maintain a requested indoor environmental atmosphere. Thus, it would be desirable to optimize ventilation rates in a manner that optimizes both the concentration of the one or more atmospheric component of the enclosure, and energy use of the enclosure (e.g., of the ventilation system servicing the enclosure).

In some embodiments, a ventilation system (e.g., as part of an HVAC system) supplies conditioned, fresh, external, and/or recirculated atmosphere to an enclosure. The ventilation system may include a heat pump and/or gas (e.g., air) handler. The gas handler may include one or more blowers (e.g., single speed or variable speed), one or more mixing chambers, one or more filters, one or more dampers, and/or one or more ducts. The ventilation system may deliver conditioned atmosphere to an enclosure (e.g., a room such as an office, a conference room, a cafeteria, a corridor, an elevator, or a lobby) via delivery and/or return ducts. In some embodiments, one or more sensors or sensor ensembles in an enclosure are configured to (e.g., and do) measure concentration of one or more atmospheric components, room occupancy, and/or ventilation flow rate. In some embodiments, sensed (e.g., measured) quantities are utilized to estimate the concentration of atmospheric components, zone (e.g., room) occupancy, or ventilation rate. A control system may use the sensed and/or the estimated concentration of the (i) atmospheric component, (ii) occupancy, and/or (iii) ventilation rate, together with knowledge of an outside (fresh air) concentration of the atmospheric component(s), to issue commands to the ventilation system. The commands to issue to the ventilation system may be for adjusting ventilation rates to optimize atmospheric quality in the enclosure, and energy usage of the enclosure (e.g., of the ventilation system servicing the enclosure). In some embodiments, combining atmospheric components(s) (such as VOC, particulate matter, or CO₂) detection and occupancy detection enables calculation of an existing ventilation rate and estimating what ventilation rate is needed to purge stale atmosphere in a given amount of time. The particulate matter may comprise particles associated with smoke and/or soot (e.g., having a FLS of at most one micrometer). The particulate matter sensor may be utilized to detect smoke and/or fire in the facility (or in the vicinity thereof). Particulate matter may affect the air quality (e.g., per air quality index). A rate of change in the atmospheric component(s) can be used to predict future levels and proactively control ventilation (with or without taking occupancy into account). Furthermore, by obtaining indoor and outdoor measurements of particulate matter, a filter efficiency can be evaluated in order to detect a need for any filter changing and/or pathogen buildup.

In some embodiments, the particulate matter sensor may use sensing an optical density of a body of gas (e.g., air), e.g., through which an energy beam travels. The particular matter sensor may measure a dispersion (e.g., dispersion pattern) of the energy beam as it travels through the body of gas. The particulate matter sensor may measure an intensity (e.g., an optical density) of the energy beam after it has passed through the body of gas, e.g., as compared to that of the energy beam as it is entering into the body of gas (e.g., as it is emitted from the energy source such as from a laser). The particulate matter sensor may utilize an energy beam that travels through a body of gas, e.g., and is dispersed on encountering a particulate matter in that body of gas (e.g., air). The energy beam may comprise a laser beam. The laser beam may be configured to an energy of at least 500 nanometers (nm), 525 nm, 550 nm, 600 nm, 650 nm, 660 nm, 700 nm, 750 nm, or 800 nm. The energy beam may comprise an infrared (IR) energy beam. The particulate matter may sense at a frequency of every 1 second (sec), 2.5 sec, 5 sec, 7.5 sec, 10 sec, 20 sec, 30 sec, or 60 sec. The particulate matter may be configured to sense at least nanometer, or micrometer sized particles. The particulate matter sensed by a particulate matter sensor may comprise particles of a FLS (e.g., diameter or diameter of its bounding circle) of at least a nanometer or a micrometer scale. For example, the particulate matter sensed by the particulate matter sensor may be of a FLS of at least 1 micrometer (μm), 2 μm, 2.5 μm, 5 μm, 7 μm, 10 μm, or 20 μm. The particulate matter sensed by the particulate matter sensor may be of any value between the aforementioned values, e.g., from about 1 μm (PM₁) to about 20 μm (PM₂₀), from about 1 μm (PM₁) to about 5 μm (PM₅), from about 2.5 μm (PM_{2.5}) to about 10 μm (PM₁₀), or from about 5 μm (PM₅) to about 20 μm (PM₂₀). The particulate matter sensor alone or in conjunction with data of other sensor(s) (e.g., VOC sensor, light sensor, noise sensor, and/or personnel ID sensor) may be utilized to monitor, notify, and/or optimize cleaning service in a facility. For example, the sensor(s) may be utilized to alert that a cleaning service is required in a portion (e.g., an enclosure) of a facility, e.g., based on sensing elevated foul odor, elevated particulate matter, and/or high number of personnel (e.g., beyond a threshold value, and/or as a function of time such as at a certain timespan). For example, the sensor(s) may be utilized to alert that a cleaning service is taking place in a portion (e.g., an enclosure) of a facility, e.g., based on sensing elevated VOC levels associated with cleaning supplies and/or particulate matter emitted during cleaning, noise of the cleaning machine, sensing ID of the cleaning personnel, and/or turning light on an off as the cleaning personnel passes through the facility. Such monitoring may allow cleaning a facility on demand, e.g., based on sensor(s), e.g., as opposed to following a scheduled cleaning service that is not sensitive to the degree of cleaning required. Such sensor(s) may also allow monitoring the rate of cleaning, certain aspects regarding the manner of cleaning (e.g., level of cleaning supplies utilized, time it takes to clean certain areas of the facility, sequence of cleaning, cleaning path, or any combination thereof). The sensor(s) (e.g., along or in synergy) may be utilized to detect odor detection in a facility (e.g., enclosure thereof such as a restroom or an office). The enclosure may constitute a space type, e.g., any space type disclosed herein. The odor may comprise volatile organic compound(s). The synergy may be of data from one sensor type with data from other sensor type(s). The synergy may be of data from one sensor type with data from other sensor of the same type. At least two of the sensor types may be disposed at (e.g., approximately) the same location, e.g., as part of a device ensemble. At least two of the sensor types may be disposed at different locations. The sensor(s) may be disposed internally in the facility (e.g., in the enclosure).

In some embodiments, an occupant in a zone is discovered and/or located via locating technology (e.g., auto-location technology). At least a portion of the locating technology may be embedded in an identification tag of an occupant (e.g., as a microchip). In some embodiments, and identification (ID) tag of a user can include a micro-chip. The micro-chip can be a micro-location chip. The micro-chip can incorporate auto-location technology (referred to herein also as “micro-location chip”). The micro-chip may incorporate technology for automatically reporting high-resolution and/or high accuracy location information. The auto-location technology can comprise Global Positioning System (GPS), Bluetooth, or radio-wave technology. The auto-location technology can comprise electromagnetic wave (e.g., radio wave) emission and/or detection. The radio-wave technology may be any RF technology disclosed herein (e.g., high frequency, ultra-high frequency, super high frequency. The radio-wave technology may comprise UWB technology. The micro-chip may facilitate determination of its location within an accuracy of at most about 25 centimeters, 20 cm, 15 cm, 10 cm, or 5 cm. In various embodiments, the control system and/or antennas (that are operatively coupled to the network) are configured to communicate with the micro-location chip. In some embodiments, the ID tag may comprise the micro-location chip. The micro-location chip may be configured to broadcast one or more signals. The signals may be omnidirectional signals. One or more component operatively coupled to the network may (e.g., each) comprise the micro-location chip. The micro-location chips (e.g., that are disposed in stationary and/or known locations) may serve as anchors. By analyzing the time taken for a broadcast signal to reach the anchors within the transmittable distance of the ID tag, the location of the ID tag may be determined. One or more processors (e.g., of the control system) may perform an analysis of the location related signals. For example, the relative distance between the micro-chip and one or more anchors and/or other micro-chip(s) (e.g., within the transmission range limits) may be determined. The relative distance, location, and/or anchor information may be aggregated. At least one of the anchors may be disposed in a floor, ceiling, wall, and/or mullion of a building. There may be at least 1, 2, 3, 4, 5, 8, or 10 anchors disposed in the enclosure (e.g., in the room, in the building, and/or in the facility). At least two of the anchors may have at least of (e.g., substantially) the same X coordinate, Y coordinate, and Z coordinate (of a Cartesian coordinate system).

In some embodiments, a control system enables locating and/or tracking one or more devices (e.g., comprising auto-location technology such as the micro location chip) and/or at least one user carrying such device. The relative location between two or more such devices can be determined from information relating to received transmissions, e.g., at one or more antennas and/or sensors. The location of the device may comprise geo-positioning and/or geolocation. The location of the device may an analysis of electromagnetic signals emitted from the device and/or the micro-location chip. Information that can be used to determine location includes, e.g., the received signal strength, the time of arrival, the signal frequency, and the angle of arrival. When determining a location of the one or more devices from these metrics, a triangulation module may be implemented. The triangulation module may comprise a calculation and/or algorithm. The triangulation may account for and/or utilize the physical layout of a building. The auto-location may comprise geolocation and/or geo-positioning. Examples of location methods may be found in International Patent Application Serial No. PCT/US17/31106, filed May 4, 2017, titled “WINDOW ANTENNAS,” which is incorporated herein by reference in its entirety.

In some embodiments, pulse-based ultra-wideband (UWB) technology (e.g., ECMA-368, or ECMA-369) is a wireless technology for transmitting large amounts of data at low power (e.g., less than about 1 millivolt (mW), 0.75 mW, 0.5 mW, or 0.25 mW) over short distances (e.g., of at most about 300 feet (′), 250′, 230′, 200′, or 150′). The short distances can be of at most about 100 meters (m), 90 m, 80 m, 70 m, 60 m, 50 m, 40 m, 30 m, 20 m, 15 m, 10 m or 5 m. A UWB signal can occupy at least about 750 MHz, 500 MHz, or 250 MHz of bandwidth spectrum, and/or at least about 30%, 20%, or 10% of its center frequency. The UWB signal can be transmitted by one or more pulses. A component broadcasts digital signal pulses may be timed (e.g., precisely) on a carrier signal across a number of frequency channels at the same time. Information may be transmitted, e.g., by modulating the timing and/or positioning of the signal (e.g., the pulses). Signal information may be transmitted by encoding the polarity of the signal (e.g., pulse), its amplitude and/or by using orthogonal signals (e.g., pulses). The UWB signal may be a low power information transfer protocol. The UWB technology may be utilized for (e.g., indoor) location applications. The broad range of the UWB spectrum comprises low frequencies having long wavelengths, which allows UWB signals to penetrate a variety of materials, including various building fixtures (e.g., walls). The wide range of frequencies, including the low penetrating frequencies, may decrease the chance of multipath propagation errors (without wishing to be bound to theory, as some wavelengths may have a line-of-sight trajectory). UWB communication signals (e.g., pulses) may be short (e.g., of at most about 70 cm, 60 cm, or 50 cm for a pulse that is about 600 MHz, 500 MHz, or 400 MHz wide; or of at most about 20 cm, 23 cm, 25 cm, or 30 cm for a pulse that is has a bandwidth of about 1 GHz, 1.2 GHz, 1.3 GHZ, or 1.5 GHz). The short communication signals (e.g., pulses) may reduce the chance that reflecting signals (e.g., pulses) will overlap with the original signal (e.g., pulse).

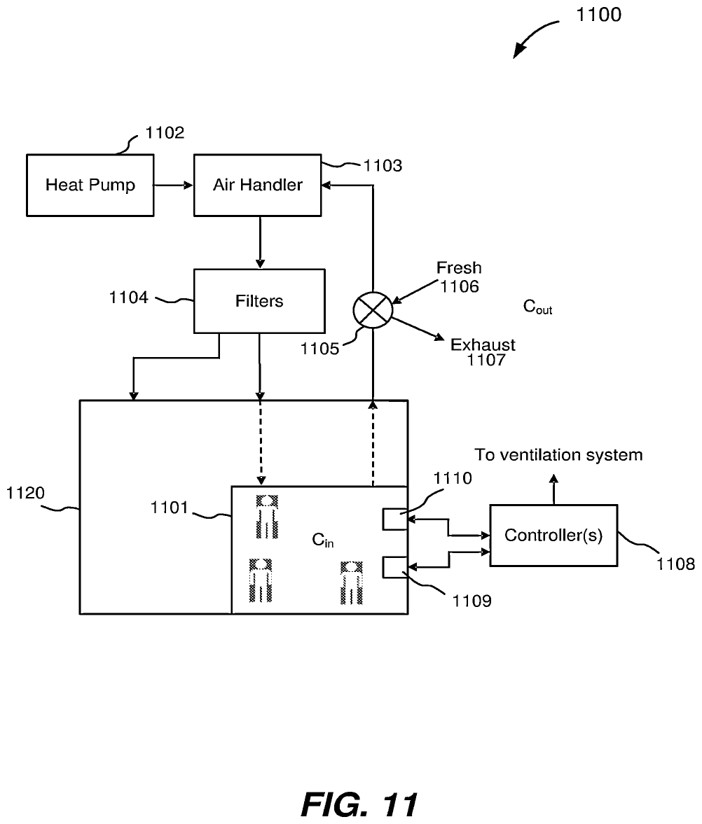

FIG. 11 depicts a ventilation system 1100 for ventilating an enclosure (e.g., room) 1101 inside a building 1120. A heat pump 1102 provides a heated or cooled heat exchange media to a gas handling system having blowers 1103, filters 1104, and mixing chamber 1105. After filtration, conditioned atmosphere is delivered to enclosure 1101 and mixes with the atmosphere in enclosure 1101 resulting in an inside atmospheric component concentration Cᵢₙ. Return atmosphere from enclosure 1101 is delivered to mixing chamber 1105 where some or all may be directed to an exhaust 1107 and replaced by fresh atmosphere (e.g., air) 1106 having an ambient outside atmospheric component concentration Cₒᵤₜ. A controller 1108 may be part of a controller network in building 1120 for controlling, e.g., one or more devices (e.g., tintable windows) and/or other aspects of a BMS. Controller 1108 is coupled to sensors 1109 and 1110 deployed in enclosure 1101 to monitor environmental characteristics such as atmosphere component concentration (e.g., CO₂, VOC, and/or particulate matter concentration). Controller 1108 may be configured to perform operations that identify adjustments to a ventilation rate that optimizes atmospheric component concentration and atmosphere quality, and the adjustments are transmitted to the ventilation system 1100 (e.g., directly or via a BMS).

Industry standards (e.g., from the American Society of Heating, Refrigerating and Air-Conditioning Engineers under ANSI) recommend a minimum ventilation rate defined according to a size (e.g., floor space or room volume) of an enclosure (e.g., room), enclosure occupancy, and use case (e.g., an office). Occupancy and/or use case may indicate a requested level of the creation of atmospheric components (e.g., pollutants) (e.g., such as CO₂, hydrogen, methane, and/or VOCs) generated in the room. Occupancy and/or use case may indicate a requested level of any required components (e.g., oxygen and/or humidity). A mass balance equation can be used to calculate a necessary ventilation rate (e.g., including intake of external atmosphere (e.g., fresh air)) to maintain a requested concentration in the room. The external atmosphere may have a lower concentration of the atmospheric component (e.g., accumulant or depletant) as compared to the concentration of that component in the atmosphere of the enclosure. The external atmosphere may have a higher concentration of the atmospheric component (e.g., humidity) as compared to the concentration of that component in the atmosphere of the enclosure. A target (e.g., optimum such as maximum or minimum) concentration of the atmospheric component to be maintained may be different (e.g., higher or lower) than an outdoor concentration. For an accumulant (e.g., VOC, or CO₂) the target may be a maximum optimum. For a depletant (e.g., O₂), the target may be a minimum optimum. At a maximum room occupancy, a minimum ventilation rate can be determined, e.g., by considering the standard recommendations and/or ventilation rate lookup table, so that the target concentration of the component is maintained at or below a threshold. The threshold can be a value, or a function (e.g., temperature dependent function). The target concentration may be specified in terms of a differential concentration (ΔPOL) between in the internal concentration of the component in the enclosure (Cᵢₙ) and an external concentration of the component out of the enclosure (Cₒᵤₜ), e.g., concentration in the ambient atmosphere. If the minimum ventilation rate for maximum occupancy is maintained during times of lower occupancy within the room, then over-ventilation is likely to occur. The (e.g., health and/or jurisdictional) standards may recommend a lower minimum ventilation rate threshold, e.g., for lower occupancy levels within the enclosure (e.g., room). However, such recommendations may over-ventilate the enclosure (even at the lower occupancy levels). Thus, an accurate ventilation rate that relies of (e.g., real time and/or in-situ) sensor measurements may provide a more accurate guideline, may facilitate reduction of energy (e.g., of the ventilation system), and/or may facility reduction in operational cost (e.g., ventilation cost) as compared to following the guidelines. The lookup table may consider (and/or delineate) the zone type (e.g., building part type such as office, conference room, corridor, lobby, etc.), relative geographical location of the zone (e.g., in relation to the sun and/or building), weather condition, zone surface area, zone volume, zone temperature, and/or expected activity in the zone (e.g., exercise in a gym, eating in a cafeteria, talking in a conference room, quiet work in an office). Data in the lookup table may be utilized to estimate the requested ventilation rate. For example, more oxygen is consumed by occupants of a gym as compared to those of an office of the same (e.g., approximate) size. For example, more humidity, VOC, and CO₂ are expelled by occupants of a gym as compared to occupants of an office of the same (e.g., approximate) size. For example, more VOCs are expelled by occupants and/or become otherwise volatile in a hot room (e.g., in a south directed room), than in a cooler room (e.g., in a north directed room).

In some embodiments, at least one of the atmospheric components is a VOC. The atmospheric component (e.g., VOC) may include benzopyrrole volatiles (e.g., indole and skatole), ammonia, short chain fatty acids (e.g., having at most six carbons), and/or volatile sulfur compounds (e.g., Hydrogen sulfide, methyl mercaptan (also known as methanethiol), dimethyl sulfide, dimethyl disulfide and dimethyl trisulfide). The atmospheric component (e.g., VOC) may include 2-propanone (acetone), 1-butanol, 4-ethyl-morpholine, Pyridine, 3-hexanol, 2-methyl-cyclopentanone, 2-hexanol, 3-methyl-cyclopentanone, 1-methyl-cyclopentanol, p-cymene, Octanal, 2-methyl-cyclopentanol, Lactic acid, methyl ester, 1,6-heptadien-4-ol, 3-methyl-cyclopentanol, 6-methyl-5-hepten-2-one, 1-methoxy-hexane, Ethyl (−)-lactate, Nonanal, 1-octen-3-ol, Acetic acid, 2,6-dimethyl-7-octen-2-ol (dihydromyrcenol), 2-ethyl hexanol, Decanal, 2,5-hexanedione, 1-(2-methoxypropoxy)-2-propanol, 1,7,7-trimethylbicyclo[2.2.1]heptan-2-one (camphor), Benzaldehyde, 3,7-dimethyl-1,6-octadien-3-ol (linalool), 1-methyl hexyl acetate, Propanoic acid, 6-hydroxy-hexan-2-one, 4-cyanocyclohexene, 3,5,5-trimethylcyclohex-2-en-1-one (isophoron), Butanoic acid, 2-(2-propyl)-5-methyl-1-cyclohexanol (menthol), Furfuryl alcohol, 1-phenyl-ethanone (acetophenone), Isovaleric acid, Ethyl carbamate (urethane), 4-tert-butylcyclohexyl acetate (vertenex), p-menth-1-en-8-ol (alpha-terpineol), Dodecanal, 1-phenylethylester acetic acid, 2 (5H)-furanone, 3-methyl, 2-ethylhexyl 2-ethylhexanoate, 3,7-dimethyl-6-octen-1-ol (citronellol), 1,1′-oxybis-2-propanol, 3-hexene-2,5-diol, 3,7-dimethyl-2,6-octadien-1-ol (geraniol), Hexanoic acid, Geranylacetone³, 2,4,6-tri-tert-butyl-phenol, Unknown, 2,6-bis(1,1-dimethylethyl)-4-(1-oxopropyl) phenol, Phenyl ethyl alcohol, Dimethylsulphone^{c}, 2-ethyl-hexanoic acid, Unknown, Benzothiazole, Phenol, Tetradecanoic acid, 1-methylethyl ester (isopropyl myristate), 2-(4-tert-butylphenyl) propanal (p-tert-butyl dihydrocinnamaldehyde), Octanonic acid, α-methyl-β-(p-tert-butylphenyl) propanal (lilial), 1,3-diacetyloxypropan-2-yl acetate (triacetin), p-cresol, Cedrol, Lactic acid, Hexadecanoic acid, 1-methylethyl ester (isopropyl palmitate), 2-hydroxy, hexyl ester benzoic acid (hexyl salicylate), Palmitic acid, ethyl ester, Methyl 2-pentyl-3-oxo-1-cyclopentyl acetate (methyl dihydrojasmonate or hedione), 1,3,4,6,7,8-hexahydro-4,6,6,7,8,8-hexamethyl-cyclopenta-gamma-2-benzopyran (galaxolide), 2-ethylhexylsalicylate, Propane-1,2,3-triol (glycerin), Methoxy acetic acid, dodecyl ester, α-hexyl cinnamaldehyde, Benzoic acid, Dodecanoic acid, 5-(hydroxymethyl)-2-furaldehyde, Homomethylsalicylate, 4-vinyl imidizole, Methoxy acetic acid, tetradecyl ester, Tridecanoic acid, Tetradecanoic acid, Pentadecanoic acid, Hexadecanoic acid, 9-hexadecanoic acid, Heptadecanoic acid, 2,6,10,15,19,23-hexamethyl-2,6,10,14,18,22-tetracosahexaene (squalene), Hexadecanoic acid, and/or 2-hydroxyethylester.

In some embodiments, sensor data (e.g., both indoor and outdoor) for atmospheric component(s) of interest (e.g., depletant such as O₂, accumulant such as CO₂) is used in conjunction with occupancy sensor, to estimate a level of the atmospheric component(s) in an enclosure, and/or distribution of the atmospheric component(s) in the enclosure. Sensors (e.g., differential pressure sensors) for measuring ventilation flow rates in ducts and/or into particular rooms, are not used due to high cost and/or low accuracy. Even when present, a gas flow and/or a pressure sensor cannot detect the makeup of the gas(es) (e.g., that arrive from an ambient outdoor environment and/or are recirculated in the enclosure). Absence of gas makeup detection hinders and/or compromises determination of (i) the actual accurate flow rate of external atmosphere (e.g., fresh air) into the enclosure, and/or (ii) the quality of the atmosphere in the enclosure (e.g., at a given time). The enclosure, a portion of the enclosure, or a group of enclosures, can define a zone. In some embodiments, inhabitant population of a zone, area and/or volume of the zone, and typical per-person generation and/or consumption rates of the atmospheric component(s) are used to calculate a difference between atmospheric component(s) levels inside the zone and atmospheric component(s) levels outside of the zone. The zone can be an enclosure. Some atmospheric components of interest are accumulants as occupants expel them. Some atmospheric components of interest are depletants as occupants consume and thus deplete them. An assumption may be used that each person expels an average atmospheric component(s) of interest (e.g., VOC, and/or CO₂) rates. An assumption may be used that each person consumes an average atmospheric component(s) of interest (e.g., O₂) rates.

In some embodiments, room occupancy, ventilation rate, and ΔPOL for one or more component(s) are related such that any one of them can be derived (e.g., calculated) from the other two. In some embodiments, occupancy (n) is calculated from measured ΔPOL (e.g., ΔCO₂ or ΔVOC) and known gas-flow rate. In some embodiments, ΔPOL is determined (e.g., calculated) from known gas-flow rate and occupancy data. The occupancy data may be detected occupancy (e.g., using an occupancy sensor). The occupancy data may consider a schedule. The occupancy data may consider historical occupancy data and/or predictive logic (e.g., using learning algorithm(s)). The learning algorithms may utilize historic data, and/or projected schedule as a learning set to predict occupancy in the enclosure. The predicted occupancy may be based on a schedule (e.g., calendar) for the enclosure and/or for the facility in which the enclosure is disposed. The schedule may be an electronic schedule. The schedule may be considered by the control system. In some embodiments, a gas-flow rate is determined from occupancy data (e.g., detected and/or projected) and measure ΔPOL. In some embodiments, once all three parameters are obtained, they can be used for regulating (e.g., with increased accuracy) ventilation rate and/or atmospheric (e.g., air) quality in the zone (e.g., enclosure). In some embodiments, measured and/or determined (e.g., calculated) values of ΔPOL (e.g., alone) are used for gross adjustment of ventilation rate (e.g., either increased or decreased rate) according to whether the actual ΔPOL is greater than or less than the target ΔPOL.

In some embodiments, a control system (e.g., comprising a processor) is adapted to control, identify and/or implement changes in a ventilation rate. Control identification and/or implementation can be according to one or more of the relationships delineated herein, e.g., between zone occupancy, ventilation rate, and ΔPOL. The control system (e.g., a controller and/or processor thereof) may store and/or retrieve one or more parameters and/or configuration data, e.g., depending upon the control actions to be performed and/or the sensor data available. In some embodiments, ventilation rates are controlled proactively prior to expected changes in zone (e.g., enclosure such as a room) occupancy so that atmosphere quality may be better maintained, e.g., when occupant(s) may enter or exit a room. For example, historical data recording regular fluctuations in one or more atmospheric component concentrations (e.g., ΔPOL for CO₂ and/or VOCs) can be used to anticipate regular gatherings of people. Changes in occupancy can be predicted (e.g., anticipated) based at least in part on other data sources such as an online calendar for the room or a particular person associated with the room. For example, electronic scheduling information may provide a planned meeting and attendance list. Using predicted changes in occupancy, atmospheric component generation in the room may be predicted by multiplying per person generation or consumption rates of the atmospheric component(s) according to the predicted occupancy. Prior to a substantial change in a combined atmospheric component generation/consumption rate, the ventilation rate bringing fresh atmosphere into the room may be varied to avoid spikes in the differential atmospheric component concentration(s).

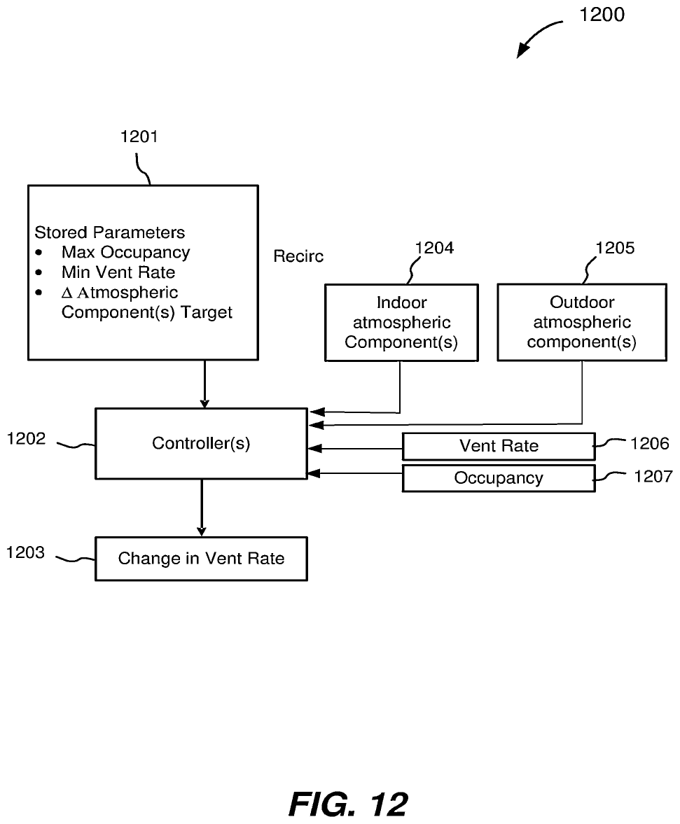

FIG. 12 depicts a control system 1200 configured to control at least ventilation. An electronic memory 1201 stores parameters such as maximum occupancy, minimum ventilation rate, maximum ventilation rate, target ventilation rate, and/or target ΔPOL. The parameter(s) are used in a control system block 1202 in an analysis (e.g., including calculations) to output a changed (e.g., altered) ventilation rate 1203. Provided that corresponding sensors are available, control system block 1202 obtains measured actual indoor (e.g., in situ) atmospheric component concentration 1204 (e.g., in real time), measured actual outdoor atmospheric component concentration 1205 (e.g., in real time), measured actual ventilation rate 1206 (e.g., in real time), and/or measured actual occupancy 1207 (e.g., in real time). When one or more sensors (e.g., sensor types) are not available to facilitate the measured data, then control system block 1202 may use available (e.g., historically measured and/or projected) values to determine (e.g., estimate and/or project) the corresponding actual values as necessary.

In some embodiments, a control system (e.g., a controller) may be configured to adapt a ventilation rate to maintain a requested atmosphere quality according to a zone (e.g., enclosure) occupancy and/or target atmospheric component levels for one or more atmospheric components. Zone occupancy may be measured, estimated, and/or determined (e.g., calculated). For example, without knowing an absolute ventilation rate, the ventilation rate can be adjusted relative to current ventilation rate, e.g., according to a difference between measured atmospheric component(s) and the target atmospheric component(s) (e.g., pollutants, depletants, and/or accumulants). A measured zone (e.g., room) occupancy may be obtained using locating technologies. The location technologies may comprise geolocation. The location technologies may utilize one or more sensors (e.g., an IR sensor array for detecting body heat signatures, a camera for identifying people using pattern recognition techniques on acquired images, or UWB tracking receivers for detect user security badges) and/or use scheduling information (e.g., an online calendar for booking a conference room). In some embodiments, the occupancy data may be used to determine a minimum ventilation rate according to an industry standard and/or other empirical relationship. While maintaining the minimum ventilation rate, one or more sensors deployed in the zone (e.g., room) may monitor atmospheric component concentration affecting atmosphere quality (e.g., O₂, CO₂, VOCs, humidity, and PM). The atmospheric component(s) may be measured both inside the enclosure and outdoors to obtain a differential concentration ΔPOL. In some embodiments, when a ΔPOL for an atmospheric component exceeds a target (e.g., optimum such as maximum or minimum) value, then the ventilation rate is increased to restore a requested atmospheric quality. The increase in ventilation rate may be proportional to the difference between the actual atmospheric component concentration and the target atmospheric component concentration. The increase in ventilation rate may be a predetermined step size. In some embodiments, a plurality (e.g., two or more) of atmospheric components can be controller in the zone (e.g., in the enclosure). The at least two of the plurality of atmospheric components can be controller simultaneously. The at least two of the plurality of atmospheric components can be controller consecutively. At least one of the plurality of atmospheric components can be controller continuously. At least one of the plurality of atmospheric components can be controller intermittently. One or more of the plurality of atmospheric components can be included into recommended changes in the ventilation rate for the zone (e.g., enclosure). Standard ventilation rates relating to one or more of the plurality of atmospheric components can be considered while formulating the recommended changes in the ventilation rate for the zone (e.g., enclosure). When atmospheric components (e.g., or standard ventilation rates thereof) are considered when formulating any change to the ventilation rate, at least two of the atmospheric components can have (e.g., substantially) the same weight, or least two of the atmospheric components can be given different weights. For example, the primary atmospheric component being controlled (e.g., monitored and/or adjusted) can be CO₂ while VOCs (from human or other sources, e.g., perspiration, aldehydes from carpet/furnishing, etc.) and/or other substances are monitored and can be given a lesser weight when included into recommended changes to the ventilation rate. The CO₂ levels can be continuously monitored and can be given the greatest weight. The VOC levels can be intermittently monitored and can be given a lesser weight as compared to the weight given to the CO₂ levels.

Determining occupancy may be performed by sensing the number of occupants in the zone using any suitable locating technology (e.g., using occupancy sensor(s)). Present occupancy or a future occupancy may be determined and/or projected (e.g., based at least in part on an electronic calendar, historical data, and/or learning module). The minimum ventilation rate may be determined based at least in part on the occupancy obtained. The occupancy may be used to look up a corresponding ventilation rate according to a lookup table and/or an industry standard, e.g., as applied to the dimensions and/or usage type of the enclosure. For example, for a 1000 ft² (that is about 92.9 m²) room in an office space, a total gas flow rate at maximum occupancy of 60 people using CO₂ as the controlled variable may be as follows: Total Gas flow=7.5×60+0.06×1000=510 cfm (that is about 896 m³/h). A maximum atmospheric component concentration occurs at this maximum occupancy as follows: max (ΔCO₂)=60×10500/510=1235 ppm. Taking outside ambient concentration for CO₂ at 400 ppm, a maximum absolute indoor concentration (C_{design}) is as follows: C_{design}=ΔCO₂+Cₒᵤₜ=1235+400=1635 ppm. At a lower room occupancy (e.g., 28 people), the industry standard minimum ventilation rate is: Total Gas flow=7.5×28+0.06×1000=270 cfm. Thus, a ventilation rate command for 270 cfm (that is about 459 m³/h) can be sent to the ventilation system. A steady state differential concentration of CO₂ under this occupancy and ventilation rate is: ΔCO₂=28×10500/270=1089 ppm. In the event that a higher atmosphere quality (lower concentration of the atmospheric component) is requested, then an incremental ventilation rate may be requested. For example, in order to limit the differential ΔCO₂ to a value of 800 ppm, the ventilation rate determined above for 28 people would be increased according to a ratio of the differential concentrations as follows: Requested Gas flow=270×(1089/800)=368 cfm. Thus, the ventilation rate would be incremented to 368 cfm (that is about 625 m³/h) for the higher atmosphere quality.

In some embodiments, a ventilation rate is controlled in response to occupancy, a maximum or target atmospheric component concentration, and an actual atmospheric component concentration. For example, ventilation rate may be adjusted up or down in order to provide an exchange of fresh atmosphere into a zone (e.g., an enclosure) to maintain the requested atmospheric component concentration without knowing a proportion of fresh to recirculated atmosphere being supplied, without requiring numerical determination of the actual ventilation rate, and/or without measurement of the actual ventilation rate. In some embodiments, occupancy is measured using at least one sensor operatively (e.g., communicatively coupled) to a network of the building. The at least one of the sensor(s) can be mounted in a sensor ensemble (e.g., a networked module integrating sensor(s), emitter(s) and/or actuator(s)) that may include atmospheric component sensor(s) (e.g., CO₂ sensor, VOC sensor, humidity sensor, oxygen sensor, and/or PM sensor). In some embodiments, occupancy may be inferred (e.g., using the mass balance equation) from atmospheric component concentration measurements, e.g., if an actual fresh atmosphere ventilation rate is available. In some embodiments, an actual atmospheric component differential concentration can be estimated from a known occupancy and actual fresh atmosphere ventilation rate. The actual differential atmospheric component concentration ΔPOL may be compared to a target (e.g., maximum) ΔPOL to determine whether a current ventilation rate should be altered (e.g., increased or decreased). The alteration can be incremental, continuous, linear, or non-linear (e.g., exponential). At least two of the increments of the incremental alteration can be of the same duration. At least two of the increments of the incremental alteration can be of different durations. At least two intermissions of the incremental alteration can be of different durations. At least two intermissions of the incremental alteration can be of the same durations. The duration and/or intermissions of the incremental alteration can follow a linear or non-linear (e.g., exponential) function. If measured atmospheric component(s) ΔPOL is less than maximum ΔPOL atmospheric component(s), then the ventilation gas flow rate may be reduced. The altered gas flow rate may be set to a threshold (e.g., value) expected to reach the target ΔPOL at time t (and thereafter maintain that threshold). The altered gas flow rate may (e.g., briefly, at time<<t) deviate from the target threshold to expediate reaching the target threshold. For example, a reduced gas flow rate may be set to a value expected to reach the target ΔPOL at t (and thereafter maintain it). The reduced gas flow rate may be (e.g., briefly, at time<<t) reduced below the set threshold in order to more quickly reach the target ΔPOL. An absolute value for a target ventilation rate that would be needed to reach and maintain target concentration (e.g., maximum ΔCO₂) may be determined based at least in part on actual and/or projected occupancy. For example, a gas flow per person may be calculated by dividing the generation/consumption rate of the atmospheric component(s) by the target differential concentration (e.g., 10500/ΔCO₂), and multiply by the number of occupants in the zone (e.g., enclosure) to calculate the needed ventilation rate, with the demand being set accordingly. In some embodiments, the change in gas flow demand is proportional to a difference between the current atmospheric component(s) ΔPOL and the target ΔPOL. If measured atmospheric component(s) ΔPOL is greater than the optimum (e.g., maximum) atmospheric component(s) ΔPOL, then the ventilation gas flow rate may be increased. A selected time (t) within which a new steady state is reached can be established by controlling a transitional ventilation rate which results in an atmosphere (air) exchange rate (AER) adapted to lower the ΔPOL. For example, an AER can be calculated as follows: AER=[ln(C_{actual}/C_{design})]/t. The AER may be used to derive a transitional ventilation rate as follows: Gas flow Vt=AER×Room Volume. Using a constant transitional ventilation rate can provide a linear slope for the changing differential concentration ΔPOL. In some embodiments, an adaptive, nonlinear slope is obtained by providing a variable ventilation rate during the transition which may be less noticeable (e.g., distracting) to the occupants.

As an example of a transitional ventilation rate, a hypothetical differential concentration ΔCO₂ will be assumed of 2000 ppm with a target max (ΔCO₂) of 1235 ppm. The time to reach the target is 5 minutes. A requested atmosphere exchange rate (using an outside CO₂ of 400 ppm) is as follows: AER=[ln(2400)−ln(1635)]/5=0.077. Converting to total gas flow for a 10 foot room height in a 1000 square foot room yields: Vt=AER×Room Volume=0.077×1000×10=770 cfm. Thus, the indoor CO₂ concentration is reduced to 1635 ppm in 5 minutes using a ventilation rate of 770 cfm. Rather than a constant 770 cfm (that is about 1308 m³/h), a variable rate may be used provided that the average rate over the 5 minute period is 770 cfm.

In some embodiments, recommendations for changes in a ventilation rate are obtained by activating ventilation mechanisms (e.g., opening and closing of atmosphere handlers). The sensor(s) for measuring atmospheric component concentrations, room occupancy, ventilation pressure, and/or flow rates, may be self-contained. At least two of the sensors (e.g., of different time or of the same type) may be incorporated into a sensor ensemble. One or more sensor ensembles may be disposed in a room being controlled (e.g., monitored). A sensor ensemble may be operatively (e.g., communicatively and/or connectively) coupled to the network. The network may be operatively cooled to the control system and/or BMS. The network may be operatively coupled to the ventilation system. At least a portion of the network may comprise wires disposed in an envelope of an enclosure (e.g., building). A sensor may be configured for continuous or intermittent sensing. The continuous and/or intermittence sensing may be scheduled. For example, scheduling of the sensing can consider the past, present, and/or projected occupancy of the zone of interest. In some embodiments, a sensor ensemble is installed in a window faming (e.g., in a mullion or transom). At least a portion of the devices in the ensemble may be utilized in controlling a tintable window that is operatively coupled to the network (and therethrough to the control system). In some embodiments, the ensemble and/or window framing may incorporates an actuator (e.g., a fan or blower) configured to circulate inside atmosphere and/or exchange atmosphere between the enclosure and the outside ambient atmosphere (as an exhaust and/or an intake). Examples for ventilation system, heat management system components (e.g., fans), smart windows, networks, sensors, and control systems can be found in International Patent Application Serial No. PCT/US15/14453, filed Feb. 4, 2015, titled “FORCED AIR SMART WINDOWS,” which is incorporated herein by reference in its entirety.

In some embodiments, monitoring of atmospheric components and ventilation rates facilitates monitoring of filter efficiency. The filter efficiency may deviate due to accumulated debris (e.g., particular matter). Accumulation of debris on the filter may reduce its filtration efficiency and/or form growth media for pathogens. The efficiency of the filter may be determined using pressure sensor, gas flow sensor, time from filter installation, and/or particulate matter (PM) sensing. Atmosphere quality in an enclosure may depend on the use of filter(s) in an atmosphere handling system to remove various contaminants such as particulate matter (e.g., dust, soot, viruses, bacteria, and/or fungi). Over time, efficiency of a filter declines as it accumulates more and more particulate matter. Based at least in part on (i) knowledge of an outside PM concentration before filtering and an inside PM concentration after filtering (e.g., a differential concentration ΔPOL), (ii) a ventilation rate through the filter (e.g., total volume of contaminated atmosphere treated by the filter per unit time), (iii) time lapse from past installation, (iv) gas pressure before the filter, (v) gas pressure after the filter, (vi) filter morphology, (v) optical density of the gas before the filter, (vi) optical density of the gas after the filter, an actual filter efficiency may be determined and/or estimated. When efficiency of filtration declines below a predetermined threshold from its nominal efficiency, a user (e.g., a building manager) can be notified to perform a corrective action such as a filter replacement. A notification may be generated as a warning message delivered immediately or may be included in a periodically generated report, for example.

Sensors of a sensor ensemble may be organized into a sensor module. A sensor ensemble may comprise a circuit board, such as a printed circuit board, in which a number of sensors are adhered or affixed to the circuit board. Sensors can be removed from a sensor module. For example, a sensor may be plugged and/or unplugged from the circuit board. Sensors may be individually activated and/or deactivated (e.g., using a switch). The circuit board may comprise a polymer. The circuit board may be transparent or non-transparent. The circuit board may comprise metal (e.g., elemental metal and/or metal alloy). The circuit board may comprise a conductor. The circuit board may comprise an insulator. The circuit board may comprise any geometric shape (e.g., rectangle or ellipse). The circuit board may be configured (e.g., may be of a shape) to allow the ensemble to be disposed in a mullion (e.g., of a window). The circuit board may be configured (e.g., may be of a shape) to allow the ensemble to be disposed in a frame (e.g., door frame and/or window frame). The mullion and/or frame may comprise one or more holes to allow the sensor(s) to obtain (e.g., accurate) readings. The circuit board may include an electrical connectivity port (e.g., socket). The circuit board may be connected to a power source (e.g., to electricity). The power source may comprise renewable or non-renewable power source.

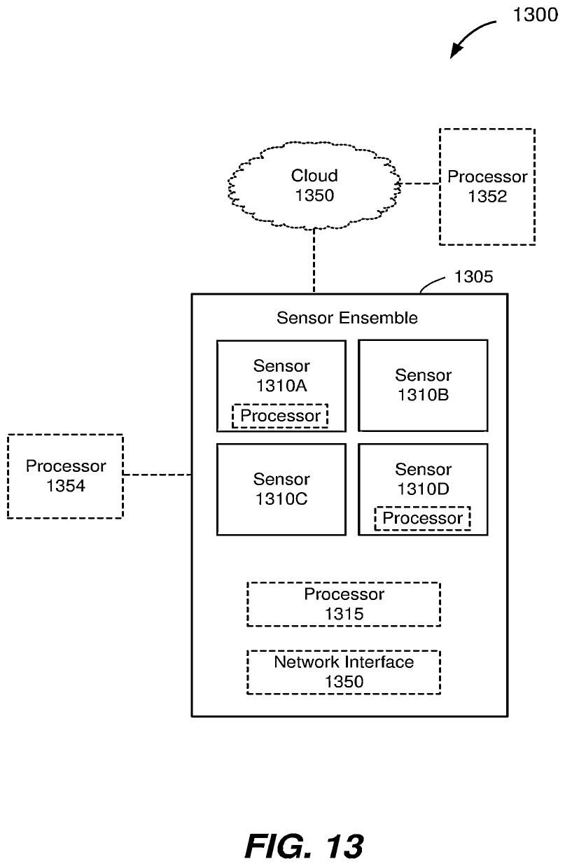

FIG. 13 is a schematic diagram depicting an example of a system having sensors of a sensor ensemble 1305 organized into a sensor module. Sensors 1310A, 1310B, 1310C, and 1310D are shown as included in sensor ensemble 1305. An ensemble of sensors organized into a sensor module may include at least 1, 2, 4, 5, 8, 10, 20, 50, or 500 sensors. The sensor module may include a number of sensors in a range between any of the aforementioned values (e.g., from about 1 to about 1000, from about 1 to about 500, or from about 500 to about 1000). Sensors of a sensor module may comprise sensors configured or designed for sensing a parameter including, for example, temperature, humidity, carbon dioxide, particulate matter (e.g., from about 2.5 μm to about 10 μm), total volatile organic compounds (e.g., via a change in a voltage potential brought about by surface adsorption of volatile organic compound), ambient light, audio noise level, pressure (e.g. gas, and/or liquid), acceleration, time, radar, lidar, radio signals (e.g., ultra-wideband radio signals), passive infrared, glass breakage, or movement. IN some cases, a sensor ensemble (e.g., sensor ensemble 1305) may include one or more non-sensor devices such as, e.g., buzzers and/or light emitting diodes. Examples of sensor ensembles and their uses can be found in U.S. patent application Ser. No. 16/447,169, filed Jun. 20, 2019, titled “SENSING AND COMMUNICATIONS UNIT FOR OPTICALLY SWITCHABLE WINDOW SYSTEMS,” that is incorporated herein by reference in its entirety.

In some embodiments, an increase in the number and/or types of sensors may be used to increase a probability that one or more measured properties is accurate and/or that a particular event measured by one or more sensor has occurred. In some embodiments, sensors of a sensor ensemble may cooperate with one another. In an example, a radar sensor of a sensor ensemble may determine presence of a number of individuals in an enclosure. A processor (e.g., processor 1315) may determine that detection of presence of a number of individuals in an enclosure is positively correlated with an increase in carbon dioxide concentration. In an example, the processor-accessible memory may determine that an increase in detected infrared energy is positively correlated with an increase in temperature as detected by a temperature sensor. In some embodiments, network interface (e.g., 1350) may communicate with other sensor ensembles similar to sensor ensemble. The network interface may additionally communicate with a controller.

Individual sensors (e.g., sensor 1310A, sensor 1310D, etc.) of a sensor ensemble may comprise and/or utilize at least one dedicated processor. A sensor ensemble may utilize a remote processor (e.g., 1354) utilizing a wireless and/or wired communications link. A sensor ensemble may utilize at least one processor (e.g., processor 1352), which may represent a cloud-based processor coupled to a sensor ensemble via the cloud (e.g., 1350). Processors (e.g., 1352 and/or 1354) may be located in the same building, in a different building, in a building owned by the same or different entity, a facility owned by the manufacturer of the window/controller/sensor ensemble, or at any other location. In various embodiments, as indicated by the dotted lines of FIG. 13, sensor ensemble 1305 is not required to comprise a separate processor and network interface. These entities may be separate entities and may be operatively coupled to ensemble 1305. The dotted lines in FIG. 13 designate optional features. In some embodiments, onboard processing and/or memory of one or more ensemble of sensors may be used to support other functions (e.g., via allocation of ensembles(s) memory and/or processing power to the network infrastructure of a building).

In some embodiments, a plurality of sensors of the same type may be distributed in an enclosure. At least one of the plurality of sensors of the same type, may be part of an ensemble. For example, at least two of the plurality of sensors of the same type, may be part of at least two ensembles. The sensor ensembles may be distributed in an enclosure. An enclosure may comprise a conference room. For example, a plurality of sensors of the same type may measure an environmental parameter in the conference room. Responsive to measurement of the environmental parameter of an enclosure, a parameter topology of the enclosure may be generated. A parameter topology may be generated utilizing output signals from any type of sensor of sensor ensemble, e.g., as disclosed herein. Parameter topologies may be generated for any enclosure of a facility such as conference rooms, hallways, bathrooms, cafeterias, garages, auditoriums, utility rooms, storage facilities, equipment rooms, and/or elevators.

In particular embodiments, one or more sensors of the sensor ensemble provide readings. In some embodiments, the sensor is configured to sense a parameter. The parameter may comprise temperature, particulate matter, volatile organic compounds, electromagnetic energy, pressure, acceleration, time, radar, lidar, glass breakage, movement, or gas. The gas may comprise a Nobel gas. The gas may be a gas harmful to an average human. The gas may be a gas present in the ambient atmosphere (e.g., oxygen, carbon dioxide, ozone, chlorinated carbon compounds, or nitrogen). The gas may comprise radon, carbon monoxide, hydrogen sulfide, hydrogen, oxygen, water (e.g., humidity). The electromagnetic sensor may comprise an infrared, visible light, ultraviolet sensor. The infrared radiation may be passive infrared radiation (e.g., black body radiation). The electromagnetic sensor may sense radio waves. The radio waves may comprise wide band, or ultra-wideband radio signals. The radio waves may comprise pulse radio waves. The radio waves may comprise radio waves utilized in communication. The gas sensor may sense a gas type, flow (e.g., velocity and/or acceleration), pressure, and/or concentration. The readings may have an amplitude range. The readings may have a parameter range. For example, the parameter may be electromagnetic wavelength, and the range may be a range of detected wavelengths.

In some embodiments, the sensor data is responsive to the environment in the enclosure and/or to any inducer(s) of a change (e.g., any environmental disruptor) in this environment. The sensors data may be responsive to emitters operatively coupled to (e.g., in) the enclosure (e.g., an occupant, appliances (e.g., heater, cooler, ventilation, and/or vacuum), opening). For example, the sensor data may be responsive to an air conditioning duct, or to an open window. The sensor data may be responsive to an activity taking place in the room. The activity may include human activity, and/or non-human activity. The activity may include electronic activity, gaseous activity, and/or chemical activity. The activity may include a sensual activity (e.g., visual, tactile, olfactory, auditory, and/or gustatory). The activity may include an electronic and/or magnetic activity. The activity may be sensed by a person. The activity may not be sensed by a person. The sensors data may be responsive to the occupants in the enclosure, substance (e.g., gas) flow, substance (e.g., gas) pressure, and/or temperature.

In some embodiments, data from a sensor in a sensor in the enclosure (e.g., and in the sensor ensemble) is collected and/or processed (e.g., analyzed). The data processing can be performed by a processor of the sensor, by a processor of the sensor ensemble, by another sensor, by another ensemble, in the cloud, by a processor of the controller, by a processor in the enclosure, by a processor outside of the enclosure, by a remote processor (e.g., in a different facility), by a manufacturer (e.g., of the sensor, of the window, and/or of the building network). The data of the sensor may have a time indicator (e.g., may be time stamped). The data of the sensor may have a sensor location identification (e.g., be location stamped). The sensor may be identifiably coupled with one or more controllers.

In some embodiments, processing data derived from the sensor comprises applying one or more models. The models may comprise mathematical models. The processing may comprise fitting of models (e.g., curve fitting). The model may be multi-dimensional (e.g., two or three dimensional). The model may be represented as a graph (e.g., 2 or 3 dimensional graph). For example, the model may be represented as a contour map (e.g., as depicted in FIG. 9). The modeling may comprise one or more matrices. The model may comprise a topological model. The model may relate to a topology of the sensed parameter in the enclosure. The model may relate to a time variation of the topology of the sensed parameter in the enclosure. The model may be environmental and/or enclosure specific. The model may consider one or more properties of the enclosure (e.g., dimensionalities, openings, and/or environmental disrupters (e.g., emitters)). Processing of the sensor data may utilize historical sensor data, and/or current (e.g., real time) sensor data. The data processing (e.g., utilizing the model) may be used to project an environmental change in the enclosure, and/or recommend actions to alleviate, adjust, or otherwise react to the change.

In some embodiments, the sensor(s) are operatively coupled to at least one controller and/or processor. Sensor readings may be obtained by one or more processors and/or controllers. A controller may comprise a processing unit (e.g., CPU or GPU). A controller may receive an input (e.g., from at least one sensor). The controller may comprise circuitry, electrical wiring, optical wiring, socket, and/or outlet. A controller may deliver an output. A controller may comprise multiple (e.g., sub-) controllers. The controller may be a part of a control system. A control system may comprise a master controller, floor (e.g., comprising network controller) controller, a local controller. The local controller may be a window controller (e.g., controlling an optically switchable window), enclosure controller, or component controller. For example, a controller may be a part of a hierarchal control system (e.g., comprising a main controller that directs one or more controllers, e.g., floor controllers, local controllers (e.g., window controllers), enclosure controllers, and/or component controllers). A physical location of the controller type in the hierarchal control system may be changing. For example: At a first time: a first processor may assume a role of a main controller, a second processor may assume a role of a floor controller, and a third processor may assume the role of a local controller. At a second time: the second processor may assume a role of a main controller, the first processor may assume a role of a floor controller, and the third processor may remain with the role of a local controller. At a third time: the third processor may assume a role of a main controller, the second processor may assume a role of a floor controller, and the first processor may assume the role of a local controller. A controller may control one or more devices (e.g., be directly coupled to the devices). A controller may be disposed proximal to the one or more devices it is controlling. For example, a controller may control an optically switchable device (e.g., IGU), an antenna, a sensor, and/or an output device (e.g., a light source, sounds source, smell source, gas source, HVAC outlet, or heater). In one embodiment, a floor controller may direct one or more window controllers, one or more enclosure controllers, one or more component controllers, or any combination thereof. The floor controller may comprise a floor controller. For example, the floor (e.g., comprising network) controller may control a plurality of local (e.g., comprising window) controllers. A plurality of local controllers may be disposed in a portion of a facility (e.g., in a portion of a building). The portion of the facility may be a floor of a facility. For example, a floor controller may be assigned to a floor. In some embodiments, a floor may comprise a plurality of floor controllers, e.g., depending on the floor size and/or the number of local controllers coupled to the floor controller. For example, a floor controller may be assigned to a portion of a floor. For example, a floor controller may be assigned to a portion of the local controllers disposed in the facility. For example, a floor controller may be assigned to a portion of the floors of a facility. A master controller may be coupled to one or more floor controllers. The floor controller may be disposed in the facility. The master controller may be disposed in the facility, or external to the facility. The master controller may be disposed in the cloud. A controller may be a part of, or be operatively coupled to, a building management system. A controller may receive one or more inputs. A controller may generate one or more outputs. The controller may be a single input single output controller (SISO) or a multiple input multiple output controller (MIMO). A controller may interpret an input signal received. A controller may acquire data from the one or more components (e.g., sensors). Acquire may comprise receive or extract. The data may comprise measurement, estimation, determination, generation, or any combination thereof. A controller may comprise feedback control. A controller may comprise feed-forward control. Control may comprise on-off control, proportional control, proportional-integral (PI) control, or proportional-integral-derivative (PID) control. Control may comprise open loop control, or closed loop control. A controller may comprise closed loop control. A controller may comprise open loop control. A controller may comprise a user interface. A user interface may comprise (or operatively coupled to) a keyboard, keypad, mouse, touch screen, microphone, speech recognition package, camera, imaging system, or any combination thereof. Outputs may include a display (e.g., screen), speaker, or printer.

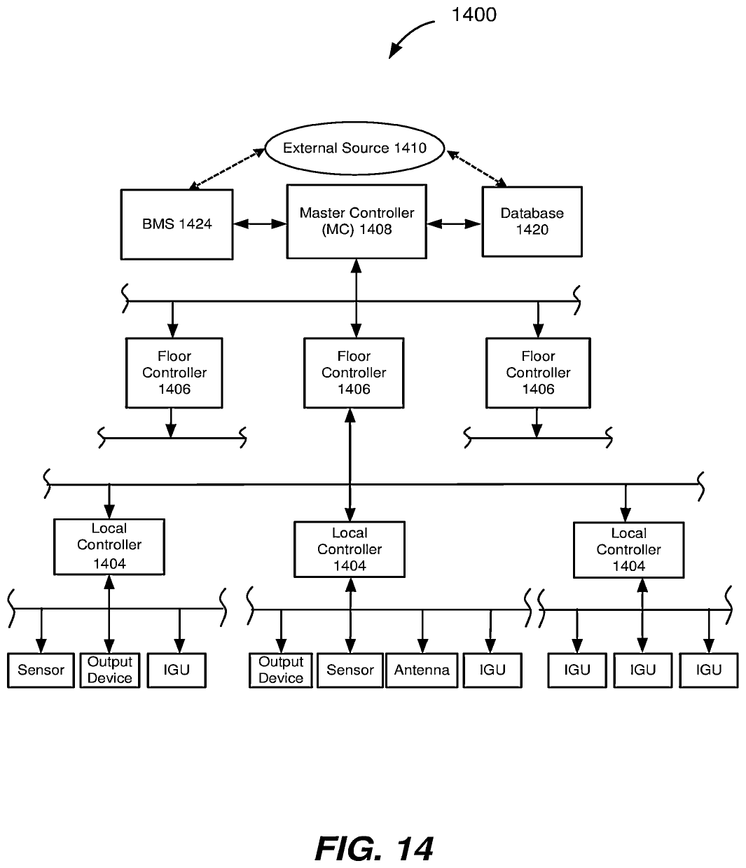

FIG. 14 shows an example of a control system architecture 1400 comprising a master controller 1408 that controls floor controllers 1406, that in turn control local controllers 1404. In some embodiments, a local controller controls one or more IGUs, one or more sensors, one or more output devices (e.g., one or more emitters), or any combination thereof. FIG. 14 shows an example of a configuration in which master controller 1408 is operatively coupled (e.g., wirelessly and/or wired) to a building management system (BMS) 1424 and to a database 1420. Arrows in FIG. 14 represents communication pathways.

A controller may be operatively coupled (e.g., directly/indirectly and/or wired and/wirelessly) to an external source (e.g., external source 1410). The external source may comprise a network. The external source may comprise one or more sensors and/or an output device. The external source may comprise a cloud-based application and/or database. The communication may be wired and/or wireless. The external source may be disposed external to the facility. For example, the external source may comprise one or more sensors and/or antennas disposed, e.g., on a wall or on a ceiling of the facility. The communication may be monodirectional or bidirectional. In the example shown in FIG. 14, the communication of all communication arrows is meant to be bidirectional.

The controller may monitor and/or direct (e.g., physical) alteration of the operating conditions of the apparatuses, software, and/or methods described herein. Control may comprise regulate, manipulate, restrict, direct, monitor, adjust, modulate, vary, alter, restrain, check, guide, or manage. Controlled (e.g., by at least one controller) may include attenuated, modulated, varied, managed, curbed, disciplined, regulated, restrained, supervised, manipulated, and/or guided. The control may comprise controlling a control variable (e.g. temperature, pressure, gas flow, occupancy, power, voltage, and/or current). The control can comprise real time or off-line control. The control can comprise in situ control. A calculation utilized by the controller can be done in real time, and/or offline. The controller may be a manual or a non-manual controller. The controller may be an automatic controller. The controller may operate upon request. The controller may be a programmable controller. The controller may be programed. The controller may comprise a processing unit (e.g., CPU or GPU). The controller may receive an input (e.g., from at least one sensor). The controller may deliver an output. The controller may comprise multiple (e.g., sub-) controllers. The controller may be a part of a control system. The control system may comprise a master controller, floor controller, local controller (e.g., enclosure controller, or window controller). The controller may receive one or more inputs. The controller may generate one or more outputs. The controller may be a single input single output controller (SISO) or a multiple input multiple output controller (MIMO). The controller may interpret the input signal received. The controller may acquire data from the one or more sensors. Acquire may comprise receive or extract. The data may comprise measurement, estimation, determination, generation, or any combination thereof. The controller may comprise feedback control. The controller may comprise feed-forward control. The control may comprise on-off control, proportional control, proportional-integral (PI) control, or proportional-integral-derivative (PID) control. The control may comprise open loop control, or closed loop control. The controller may comprise closed loop control. The controller may comprise open loop control. The controller may comprise a user interface. The user interface may comprise (or operatively coupled to) a keyboard, keypad, mouse, touch screen, microphone, speech recognition package, camera, imaging system, or any combination thereof. The outputs may include a display (e.g., screen), speaker, or printer. The methods, systems and/or the apparatus described herein may comprise a control system. The control system can be in communication with any of the apparatuses (e.g., sensors) described herein. The sensors may be of the same type or of different types, e.g., as described herein. For example, the control system may be in communication with the first sensor and/or with the second sensor. The control system may control the one or more sensors. The control system may control one or more components of a building management system (e.g., lightening, security, and/or air conditioning system). The controller may regulate at least one (e.g., environmental) characteristic of the enclosure. The control system may regulate the enclosure environment using any component of the building management system. For example, the control system may regulate the energy supplied by a heating element and/or by a cooling element. For example, the control system may regulate velocity of gas(es) flowing through a vent to and/or from the enclosure. The control system may comprise a processor. The processor may be a processing unit. The controller may comprise a processing unit. The processing unit may be central. The processing unit may comprise a central processing unit (abbreviated herein as “CPU”). The processing unit may be a graphic processing unit (abbreviated herein as “GPU”). The controller(s) or control mechanisms (e.g., comprising a computer system) may be programmed to implement one or more methods of the disclosure. The processor may be programmed to implement methods of the disclosure. The controller may control at least one component of the forming systems and/or apparatuses disclosed herein.

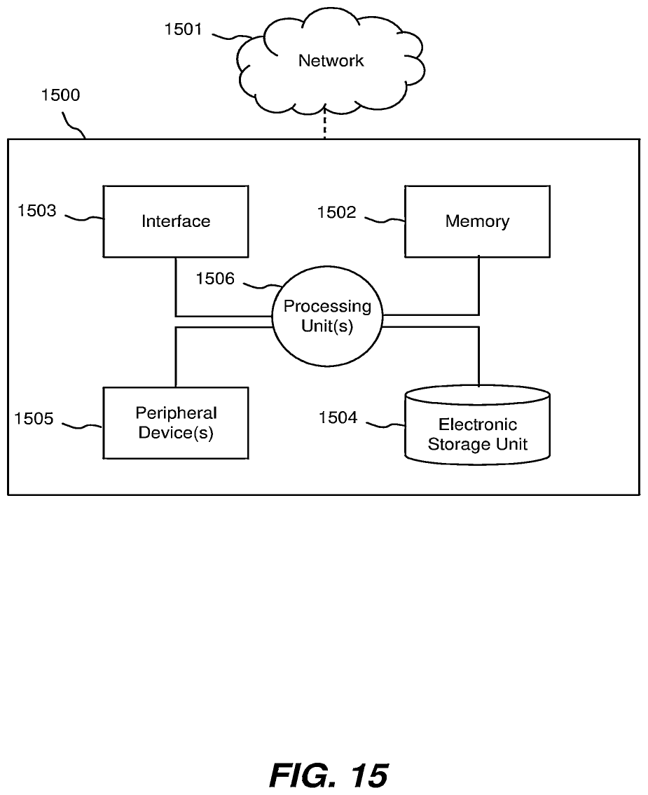

FIG. 15 shows a schematic example of a computing system 1500 that is programmed or otherwise configured to perform one or more operations of any of the methods provided herein and a network 1501. Computing system 1500 may control (e.g., direct, monitor, and/or regulate) various features of methods, apparatuses and systems described herein such as, for example, control heating, cooling, lightening, and/or venting of an enclosure, or any combination thereof. Computing system 1500 can be part of, or be in communication with, any sensor or sensor ensemble disclosed herein. Computing system 1500 may be coupled to one or more mechanisms disclosed herein, and/or any parts thereof. For example, computing system 1500 may be coupled to one or more sensors, valves, switches, lights, windows (e.g., IGUs), motors, pumps, optical components, or any combination thereof. Computing system 1500 may be an example of a controller (e.g., master controller, network controller, or local controller).

Computing system 1500 may include one or more processing units 1506 (also sometimes referred to herein as one or more processors). Computing system 1500 may also include memory or memory location 1502 (e.g., random-access memory, read-only memory, flash memory), an electronic storage unit 1504 (e.g., hard disk), a communication interface (e.g., a network adapter) for communicating with one or more other systems, and one or more peripheral devices 1505 such as, e.g., cache, other memory, data storage and/or electronic display adapters. In the example shown in FIG. 15, memory 1502, storage unit 1504, interface 1503, and peripheral devices 1505 are in communication with processing unit(s) 1506 through a communication bus (denoted by solid lines), such as a motherboard. Storage unit 1504 may be, for example, a data storage unit (or data repository) for storing data. Computing system 1500 may be operatively coupled to computer network 1501 (network) with the aid of the communication interface. Network 1501 may be the Internet, an internet and/or extranet, or an intranet and/or extranet that is in communication with the Internet. In some cases, network 1501 may be a telecommunication and/or data network. Network 1501 may include one or more computer servers, which can enable distributed computing, such as cloud computing. Network 1501, in some cases with the aid of the computer system, can implement a peer-to-peer network, which may enable devices coupled to computing system 1500 to behave as a client or a server.

Processing unit(s) 1506 can execute a sequence of machine-readable instructions, which can be embodied in a program or software. The instructions may be stored in a memory location, such as memory 1502. The instructions can be directed to processing unit(s) 1506, which can subsequently program or otherwise configure the processing unit to implement methods of the present disclosure. Examples of operations performed by processing unit(s) 1506 can include fetch, decode, execute, and write back. Processing unit(s) 1506 may interpret and/or execute instructions. Processing unit(s) 1506 may include a microprocessor, a data processor, a central processing unit (CPU), a graphical processing unit (GPU), a system-on-chip (SOC), a co-processor, a network processor, an application specific integrated circuit (ASIC), an application specific instruction-set processor (ASIPs), a controller, a programmable logic device (PLD), a chipset, a field programmable gate array (FPGA), or any combination thereof. Processing unit(s) 1506 can be part of a circuit, such as an integrated circuit. One or more other components of the computing system 1500 can be included in the circuit.

Storage unit 1504 may store files, such as drivers, libraries and saved programs. Storage unit 1504 may store user data (e.g., user preferences and user programs). In some cases, computing system 1500 may include one or more additional data storage units that are external to computing system 1500, such as located on a remote server that is in communication with computing system 1500 through an intranet or the Internet.

In some cases, computing system 1500 may communicate with one or more remote computer systems through a network. For instance, computing system 1500 may communicate with a remote computer system of a user (e.g., operator). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. A user (e.g., client) may access computing system 1500 via the network 1501.

Methods as described herein can be implemented by way of machine (e.g., computer processor) executable code stored on an electronic storage location of computing system 1500, such as, for example, on memory 1502 or electronic storage unit 1504. The machine executable or machine-readable code can be provided in the form of software. During use, processing unit(s) 1506 may execute the code. In some cases, the code can be retrieved from the storage unit and stored on the memory for ready access by the processor. In some situations, electronic storage unit 1504 can be precluded, and machine-executable instructions are stored on memory. The code can be pre-compiled and configured for use with a machine have a processer adapted to execute the code or can be compiled during runtime. The code can be supplied in a programming language that can be selected to enable the code to execute in a pre-compiled or as-compiled fashion.

In some embodiments, computing system 1500 comprises at least one processor (e.g., processing unit(s) 1506) that include a code. The code can be program instructions. The program instructions may cause the at least one processor (e.g., computer) to direct a feed forward and/or feedback control loop. In some embodiments, the program instructions cause the at least one processor to direct a closed loop and/or open loop control scheme. The control may be based at least in part on one or more sensor readings (e.g., sensor data). One controller may direct a plurality of operations. At least two operations may be directed by different controllers. In some embodiments, a different controller may direct at least two of operations (a), (b) and (c). In some embodiments, different controllers may direct at least two of operations (a), (b) and (c). In some embodiments, a non-transitory computer-readable medium cause each a different computer to direct at least two of operations (a), (b) and (c). In some embodiments, different non-transitory computer-readable mediums cause each a different computer to direct at least two of operations (a), (b) and (c). The controller and/or computer readable media may direct any of the apparatuses or components thereof disclosed herein. The controller and/or computer readable media may direct any operations of the methods disclosed herein.

In some embodiments, the at least one sensor is operatively coupled to a control system (e.g., computing system). The sensor may comprise light sensor, acoustic sensor, vibration sensor, chemical sensor, electrical sensor, magnetic sensor, fluidity sensor, movement sensor, speed sensor, position sensor, pressure sensor, force sensor, density sensor, distance sensor, or proximity sensor. The sensor may include temperature sensor, weight sensor, material (e.g., powder) level sensor, metrology sensor, gas sensor, or humidity sensor. The metrology sensor may comprise measurement sensor (e.g., height, length, width, angle, and/or volume). The metrology sensor may comprise a magnetic, acceleration, orientation, or optical sensor. The sensor may transmit and/or receive sound (e.g., echo), magnetic, electronic, or electromagnetic signal. The electromagnetic signal may comprise a visible, infrared, ultraviolet, ultrasound, radio wave, or microwave signal. The gas sensor may sense any of the gas delineated herein. The distance sensor can be a type of metrology sensor. The distance sensor may comprise an optical sensor, or capacitance sensor. The temperature sensor can comprise Bolometer, Bimetallic strip, calorimeter, Exhaust gas temperature gauge, Flame detection, Gardon gauge, Golay cell, Heat flux sensor, Infrared thermometer, Microbolometer, Microwave radiometer, Net radiometer, Quartz thermometer, Resistance temperature detector, Resistance thermometer, Silicon band gap temperature sensor, Special sensor microwave/imager, Temperature gauge, Thermistor, Thermocouple, Thermometer (e.g., resistance thermometer), or Pyrometer. The temperature sensor may comprise an optical sensor. The temperature sensor may comprise image processing. The temperature sensor may comprise a camera (e.g., IR camera, CCD camera). The pressure sensor may comprise Barograph, Barometer, Boost gauge, Bourdon gauge, Hot filament ionization gauge, Ionization gauge, McLeod gauge, Oscillating U-tube, Permanent Downhole Gauge, Piezometer, Pirani gauge, Pressure sensor, Pressure gauge, Tactile sensor, or Time pressure gauge. The position sensor may comprise Auxanometer, Capacitive displacement sensor, Capacitive sensing, Free fall sensor, Gravimeter, Gyroscopic sensor, Impact sensor, Inclinometer, Integrated circuit piezoelectric sensor, Laser rangefinder, Laser surface velocimeter, LIDAR, Linear encoder, Linear variable differential transformer (LVDT), Liquid capacitive inclinometers, Odometer, Photoelectric sensor, Piezoelectric accelerometer, Rate sensor, Rotary encoder, Rotary variable differential transformer, Selsyn, Shock detector, Shock data logger, Tilt sensor, Tachometer, Ultrasonic thickness gauge, Variable reluctance sensor, or Velocity receiver. The optical sensor may comprise a Charge-coupled device, Colorimeter, Contact image sensor, Electro-optical sensor, Infra-red sensor, Kinetic inductance detector, light emitting diode (e.g., light sensor), Light-addressable potentiometric sensor, Nichols radiometer, Fiber optic sensor, Optical position sensor, Photo detector, Photodiode, Photomultiplier tubes, Phototransistor, Photoelectric sensor, Photoionization detector, Photomultiplier, Photo resistor, Photo switch, Phototube, Scintillometer, Shack-Hartmann, Single-photon avalanche diode, Superconducting nanowire single-photon detector, Transition edge sensor, Visible light photon counter, or Wave front sensor. The one or more sensors may be connected to a control system (e.g., to a processor, to a computer).

In some embodiments, measurements of one or more sensors (e.g., comprising VOC sensor(s)) may be utilized to adjust a smell (e.g., smell profile), gas borne compounds, and/or gaseous compounds of an environment. In some embodiments, gas borne comprises air borne. The smell, gas borne compounds, and/or gaseous compounds may be requested and/or preferred. The gas borne compounds may be volatile compounds. The smell may have a profile composed of one or more chemicals (e.g., gas borne chemicals). The smell may be requested and/or preferred by a user (e.g., as disclosed herein), and/or by jurisdictional (e.g., health) standard(s). The measurements of the one or more sensors may be utilized to form a sensed profile (e.g., sensed map). The profile may be as a function of space and/or time. The profile may be a two, three, or four dimensional profile. At least one of the profile data may relate to (i) space (e.g., compound(s) concentration as a function of space), and/or (ii) time (e.g., compound(s) concentration as a function of space). When the sensed profile of the chemical(s) deviates from the requested profile, the profile in the environment may be adjusted. Adjustment may be at least in part by modifying a chemical make-up of an atmosphere of the environment, changes in air flow, and/or changes in atmospheric temperature. For example, adjustment may be by adding (e.g., injecting) and/or dispersing one or more chemicals into an atmosphere. For example, adjustment may be by subtracting (e.g., expelling, extracting, or ejecting) one or more chemicals out of an atmosphere. The subtraction can be active (e.g., suction) or passive (e.g., absorption). At least one of the adjusted chemical(s) may be the same as the sensed chemical(s) found as deficient. At least one of the adjusted chemical(s) may be different from the sensed chemical(s) found deficient. Adjustment of the chemical(s) into/out of the atmosphere may occur when the requested chemical profile deviates from a requested chemical profile. The adjusted chemical(s) may masque the sensed chemical profile. The masking may be relative to an average user (e.g., smell that is sensed as masque by an average user). The user may be an occupant of the environment. The adjustment may be of individual compounds and/or of a mixture of compounds. The chemical(s) may be chemically identifiable or may be as part of a mixture that is not (e.g., fully) identifiable.

In some embodiments, a control system adjusts an environment based at least in part on preferences. The preferences may include (e.g., personal) preferences of a user. The preference may include jurisdictional (e.g., health) preferences, standards, and/or recommendations. A user may input an environmental preference. The environmental preference may include environmental characteristic types comprising temperature, chemical make-up of an atmosphere, gas movement velocity (e.g., ventilation speed), light intensity, or noise levels. The environmental preference may comprise rejection of one or more environmental conditions. For example, an input of the user may comprise (i) liking an environment, (ii) disliking an environment, and/or (iii) preference of a different specified environment. The specific environment may be enumerated in a menu (e.g., dropdown menu). The specific environment may be generated by the user by selection of one or more of the environmental characteristic types from a menu. An environmental characteristic type may have various levels. For example, the environmental characteristic of temperature may have various temperature levels such as about 10° C., 15° C., 20° C., 25° C., or 30° C. The chemical makeup of the atmosphere may comprise various levels (e.g., indicated as percentage or ppm) of a certain chemical (e.g., CO₂, O₂, or a particular VOC). The user may indicate a preference to a chemical makeup of the atmosphere of the enclosure. The preference may be disliking the current smell, liking the current smell, or preferring a different smell profile. The preference may be registered as user input, and coupled with a time of input entry and/or space of user entry. Various preference of the user as a function of space and/or time, may be used by the learning system as input. The learning system may use these preferences and predict future smell predictions, e.g., optionally as a function of space. The learning system may use preferences of a plurality of users (e.g., a group of users) and predict future smell predictions, e.g., optionally as a function of space and/or space types. The users may occupy the space adjacent to each other (e.g., in one open space region). The users may occupy similar space types. The space types may comprise similar type of rooms such as office rooms, conference rooms, break-rooms, cafeterias, corridors, bathrooms, or elevators. The space types may be defined and/or identified, e.g., in a database. The space types may be identified by a function an occupant performs therein (e.g., studying, lecturing and/or listening to a lecture, conferring, eating, drinking, resting, secreting (e.g., urine), expelling (e.g., defecating), washing, and/or waiting).

In some embodiments, a control system adjusts an environment based at least in part on a learning scheme. The control system may be communicatively coupled to a network (e.g., as disclosed herein). The user input may be entered into a database that is operatively coupled to the network. A learning system may track the user input, e.g., as a function of space and/or time. The learning system may utilize the user input as a learning set. The learning system may form predictions(s) in a future time based at least in part of the user input. The learning system may comprise any learning scheme (e.g., algorithm) as disclosed herein. For example, the learning system may utilize an artificial intelligence scheme. In some embodiments, a control system adjusts the chemical make-up of an environment based at least in part on preferences. The preferences may include (e.g., personal) preferences of a user (e.g., an occupant). The preference may include jurisdictional (e.g., health) preferences, standards, and/or recommendations. A user may enter a smell preference. The smell preference may comprise rejection of a present smell in the environment. The smell preference may comprise liking a present smell in the environment. The smell preference may comprise indication of a requested smell in the environment (e.g., citrus smell). The control system may utilize input from at least one chemical sensor to form a present smell profile in the environment. The control system may analyze (e.g., compare) the present small profile with the requested smell profile, and generate a comparison. The smell profile may comprise indication of time, space, chemical type, and/or level of the chemical type. The control system may include one or more controllers and/or processors. The control system may analyze the comparison with respect to a threshold (e.g., value and/or function). The threshold function may be of time, space, and/or chemical type. When the comparison is greater than the threshold, the control system may adjust the smell profile of the environment by controlling a ventilation system, and/or injecting a smell component(s) (e.g., citrus smell) into the environment. The control system may utilize the learning system to anticipate requests and/or preferences of the user. The control system may automatically (e.g., without explicit user request) adjust one or more environmental characteristic based at least in part on the learning system (e.g., learning module). The user may (e.g., manually) override an environmental adjustment of the control system. Input of environmental preference of the user may be done using an application. The application may be operatively (e.g., communicatively) coupled to a mobile device. While an example of smell adjustment was provided, adjustment may be similarly done to any other atmospheric components and/or characteristic.

In some embodiments, a control system conditions various aspects of an enclosure. For example, the control system may condition an environment of the enclosure. The control system may project future environmental preferences of the user, and condition the environment to these preferences in advance (e.g., at a future time). The preferential environmental characteristic(s) may be allocated according to (i) user or group of users, (ii) time, (iii) date, and/or (iv) space. The data preferences may comprise seasonal preferences. The environmental characteristics may comprise lighting, ventilation speed, atmospheric pressure, smell, temperature, humidity, carbon dioxide, oxygen, VOC(s), particulate matter (e.g., dust), or color. The environmental characteristics may be a preferred color scheme or theme of an enclosure. For example, at least a portion of the enclosure can be projected with a preferred theme (e.g., projected color, picture, or video). For example, a user is a heart patient and prefers (e.g., requires) an oxygen level above the ambient oxygen level (e.g., 20% oxygen) and/or a certain humidity level (e.g., 70%). The control system may condition the atmosphere of the environment for that oxygen and humidity level when the heart patient occupant is in a certain enclosure (e.g., by controlling the BMS). In some embodiments, a control system may operate a component according to preference of a user or a group of users. In some embodiments, the control system may adjust the environment and/or component according to hierarchical preferences.

In some embodiments, the control system considers results (e.g., scientific and/or research based results) regarding environmental conditions that affect health, safety and/or performance of enclosure occupants. The control system may establish thresholds and/or preferred window-ranges for one or more environmental characteristic of the enclosure (e.g., of an atmosphere of the enclosure). The threshold may comprise a level of atmospheric component (e.g., VOC, particulate matter, and/or gas), temperature, and time at a certain level. The certain level may be abnormally high, abnormally low, or average. For example, the controller may allow short instances of abnormally high VOC and/or particulate matter level, but not prolonged time with that VOC and/or particulate matter level. The control system may automatically override preference of a user if it contradicts health and/or safety thresholds. Health and/or safety thresholds may be at a higher hierarchical level relative to a user's preference. The hierarchy may utilize majority preferences. For example, if two occupants of a meeting room have one preference, and the third occupant has a conflicting preference, then the preferences of the two occupants will prevail (e.g., unless they conflict health and/or safety considerations).

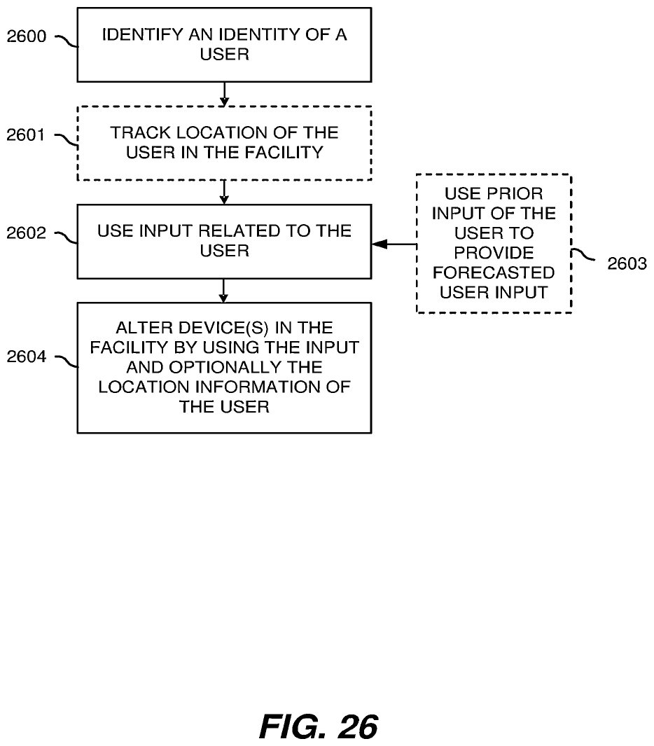

FIG. 26 shows an example of a flow chart depicting operations of a control system that is operatively coupled to one or more devices in an enclosure (e.g., a facility). In block 2600 an identify of a user is identified by a control system. The identity can be identified by one or more sensors (e.g., camera) and/or by an identification tag (e.g., by scanning or otherwise sensing by one or more sensors). In block 2601, a location of the user may optionally be tracked as the user spends time in the enclosure. The use may provide input as to any preference. The preference may be relating to a component such as a target apparatus, and/or environmental characteristics. A learning module may optionally track such preferences and provide predictions as to any future preference of the user in block 2603. Past elective preferences by the user may be recorded (e.g., in a database) and may be used as a learning set for the learning module. As the learning process progress over time and the user provides more and more inputs, the predictions of the learning module may increase in accuracy. The learning module may comprise any learning scheme (e.g., comprising artificial intelligence and/or machine learning) disclosed herein. The user may override recommendations and/or predictions made by the learning module. The user may provide manual input into the control system. In block 2602, the user input is provided (whether directly by the user or by predictions of the learning module) to the control system. The control system may alter (or direct alteration of) one or more devices in the facility to materialize the user preferences (e.g., input) by using the input. The control system may or may not use location of the user. The location may be a past location or a current location. For example, the user may enter a workplace by scanning a tag. Scanning of the identification tag (ID tag) can inform the control system of an identify of the user, and the location of the user at the time of scanning. The user may express a preference for a sound of a certain level that constitutes the input. The expression of preference may be by manual input (including tactile, voice and/or gesture command). A past expression of preference may be registered in a database and linked to the user. The user may enter a conference room at a prescheduled time. The sound level in the conference room may be adjusted to the user preference (i) when the prescheduled meeting was scheduled to initiate and/or (ii) when one or more sensors sense presence of the user in the meeting room. The sound level in the conference room may be return to a default level and/or adjusted to another's preference (i) when the prescheduled meeting was scheduled to end and/or (ii) when one or more sensors sense absence of the user in the meeting room.

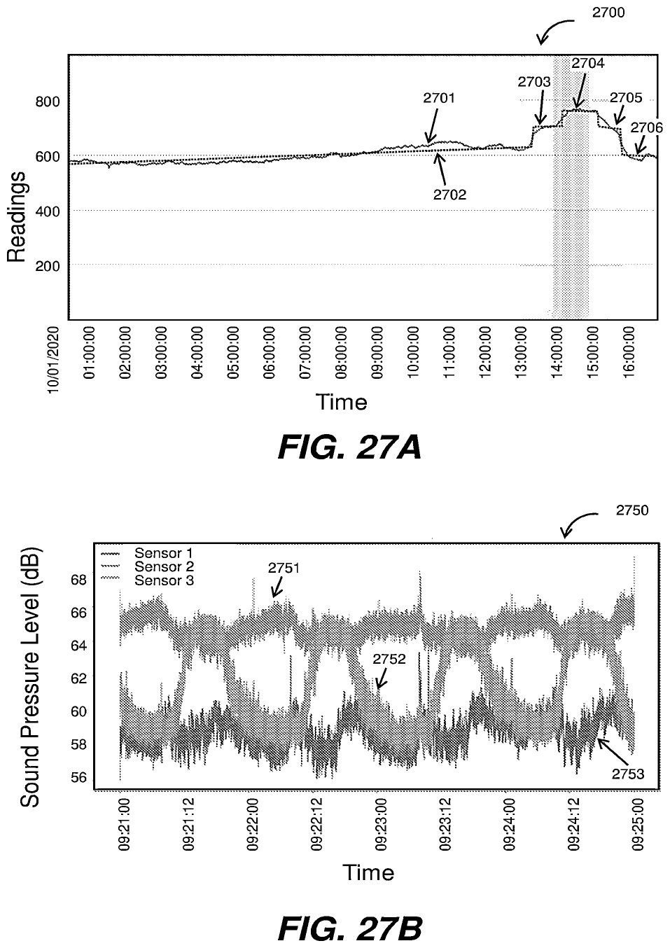

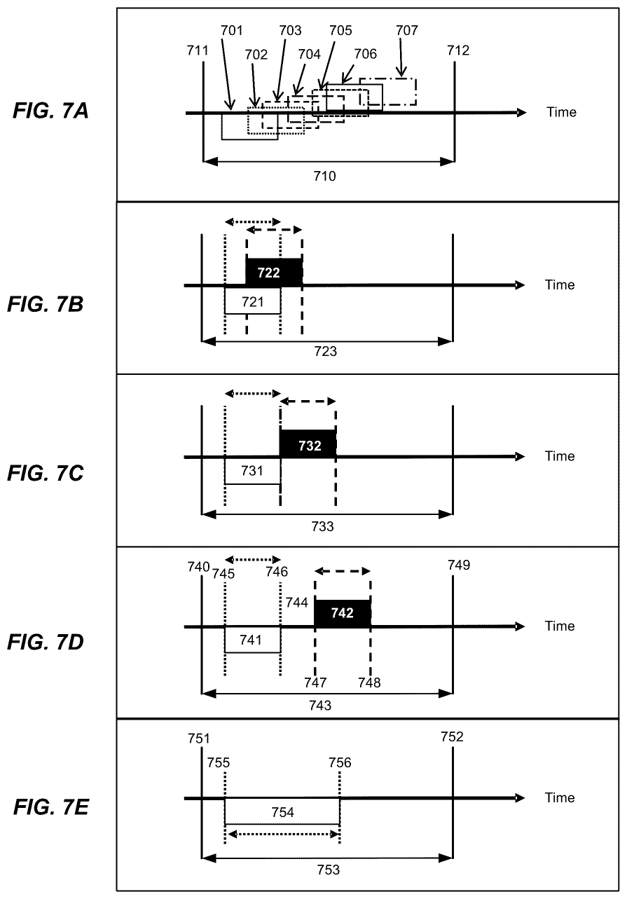

In some embodiments, detection association of personnel interaction with sensor data is obviated from the data. The sensor data may require analysis. For example, the senor data may require finding a baseline of the sensed property (e.g., sensed attribute). For example, the sensor data may require matching to a graph manipulating the data. The data manipulation may comprise filtering (e.g., high pass or low pass filtering); finding mean, average, or median; discretizing data (e.g., according to a threshold). The threshold may comprise a threshold value or a threshold function. FIG. 27A shows an example of carbon dioxide sensor data values plotted as a function of time, in graph 2700 showing sensor data 2701. An average baseline may be matched in 2702 and 2706. The carbon dioxide data may be discretized. For example, discretized values 2703, 2704, and 2705 represent discretization of the sensor data 2701. The discretization may be matched with number of personnel and/or their behavior. For example, a first person may enter a room in which the carbon dioxide sensor(s) are disposed. These sensor(s) generate data 2701. When the first person enters the room, the sensor data may elevate to a level 2703. When a second person enters the room, the sensor data may elevate to a level 2704. When the second sensor leaves the room, the sensor data may reduce to level 2703, and finally, when the first person exits the room, the sensor data will revert to the baseline level 2706. Corroboration of the entry of personnel to the room may be with other sensors. For example, ID sensor(s), or noise sensor(s). Such corroboration and/or accumulation of data over prolonged time may foresee and/or characterize behavior in that room (e.g., or in the facility). FIG. 27B shows an example of noise sensor data values plotted as a function of time, in graph 2750 showing first sensor data 2751, second sensor data 2752, and third sensor data 2753, which sensors are disposed at known and different locations in the facility. Sensor data 2751 discloses a lower noise levels as compared to sensor data 2752 that depicts a noisier environment. Sensor data 2752 depicts regular noise oscillations that could match oscillation of a motor. The level of noise can be monitored, thus obviating when a noise level is above a threshold. This provide an opportunity to alleviate such noise conditions when it arises (e.g., regardless and/or before a complaint is put forward). Such level of knowledge may provide an opportunity to monitor the motorized devices, e.g., using machine learning or another control scheme. For example, when the sound oscillation become non-repetitive, and/or exhibit another change (e.g., altered sound level, altered frequency, altered full-width-at-half-maximum (FWHM), or any combination thereof), an action may be prescribed (e.g., notification is provided). Such knowledge may allow monitoring the facility or any component (e.g., service machinery and/or production machinery) of the facility.

### Digital Twin

To address user control of conditions within a building and/or other settings and status of devices such as sensors at a facility, a digital model and associated file(s) may be associated with the facility and the one or more building systems. In certain implementations, the digital model and its associated file(s) are referred to as a “digital twin” of the facility. An example of digital twin is a Building Information Model or “BIM.” Some examples of BIM files for a BIM model include a Revit file, a Microdesk file such as a ModelStream file, an IMAGINIT file, an ATG USA file, or similar facility-related digital file. The digital twin may have associated centralized files integrating all assets at the facility, which can aid occupants and customer support personnel responsible for the facility and/or control of one or more devices within the facility. For example, the digital twin can be stored in a cloud network accessible and/or updatable by the occupants and customer support personnel.

In one aspect, a digital twin may include the location and identifiers of the devices in the building and the current settings and status of the devices in the building. The digital twin may also include user preferences such as, for example, preferred settings of one or more environmental conditions (e.g., amount of natural light in a space) and preferred settings for one or devices (e.g., transmissivity of a tintable window). In certain implementations, the digital twin of a facility may be updated to reflect real time, or substantially real time status and settings, of the devices at the facility, which can aid in deployment, maintenance, and control of the devices and environmental conditions at the facility. The digital twin can also serve as an interactive tool for customers to control in real time, or substantially real time, the environmental conditions (e.g., amount of natural light or heat load) in their space and see visualizations of their changes to environmental conditions on a three-dimensional model of the building and/or see visualizations on the three-dimensional model of the building of how their changes will cause adjustments to settings of devices in the building. For example, a customer can adjust an amount of light for their space and be provided with a visualization on a 3D model of how transmissivity of tintable windows on two adjacent facades to that space would be adjusted to accommodate their adjustment.

A digital twin may facilitate management of control of devices and building systems at various levels. In certain aspects, a digital twin may be a BIM that is supplemented with device related information such as through an app (software application). For example, input from customers (e.g., through an app) may be fed into control of the facility using the virtual building mode. The digital twin may offer a visual proofing tool prior to commissioning or for purposes of maintenance after commissioning. The digital twin may also offer a virtual reality experience of the facility (including its assets such as devices) to a user of the software application.

In some cases, a three-dimensional (3D) architectural model may be used to initialize files (e.g., BIM files) of a digital twin, to incorporate architectural elements of a facility. Ground truth validation (e.g., from a field service engineer) may be used to verify device data in the files of the digital twin. The digital twin may be initialized prior to commissioning the devices at the facility. In some cases, the initialized BIM files (such as, e.g., a Autodesk Revit file) incorporate architectural elements of a facility, but not the devices installed in the facility. The BIM files may be updated during commissioning and/or updated by the occupants and or customer support personnel.

During commissioning of devices at a facility, devices may be installed and differentiated from one another by the installer, e.g., by consulting an external label having an inscribed serial number, bar code, Quick Response (QR) code, radio frequency identification (RF ID), and/or other printed information. In some cases, the process of locating and documenting the devices during and/or after the commissioning process may be entered into the digital twin by automated and/or manual process. In some cases, commissioning may be performed to provide or correct assignment of window controller addresses and/or other identifying information to specific windows and window controllers, as well the physical locations of the windows and/or window controllers in buildings. For example, commissioning may be used to correct problems made in installing tintable windows in the wrong locations or the connecting of cables to the wrong window controllers. The commissioning process for a particular tintable window involve associating an identification (ID) for the window, or other window-related component, with a network address of its corresponding window controller. The process may (e.g., also) assign a building location and/or absolute location (e.g., latitude, longitude and/or elevation) to the window or other component.

The tintable windows (e.g., comprising electrochromic devices), electronic ensembles (e.g., containing various sensors, actuators, and/or communication interfaces), and/or associated controllers (e.g., master controllers, network controllers, and/or other controllers, e.g., responsible for tint decisions) may be interconnected in a hierarchical network, e.g., for purposes of coordinated control (e.g., monitoring). For example, one or more controllers may need to utilize the network address of the window controller(s) connected to specific windows or sets of windows. To this end, a function of commissioning may be to provide correct assignment of window controller addresses and/or other identifying information to specific windows and window controllers, as well the physical locations of the windows and/or window controllers in buildings. Another function of commissioning may be to correct the installation of windows at the wrong location or the connecting of cables to the wrong window controllers. The commissioning process for a particular window (e.g., insulated glass unit (IGU)) may involve associating an identification (ID) for the window, or other window-related component, with a network address of its corresponding window controller. The process may (e.g., also) assign a building location and/or absolute location (e.g., latitude, longitude and/or elevation) to the window or other component. Some examples to digital twins are described in PCT Application PCT/US2021/057678, filed on Nov. 2, 2021, and titled “VIRTUALLY VIEWING DEVICES IN A FACILITY,” which is hereby incorporated by reference in its entirety.

In certain aspects, a control system and/or control interface comprises, or is in communication with, a “digital twin” of a facility such as a building. For example, the digital twin may comprise a representative model (e.g., a two-dimensional or three-dimensional virtual depiction) containing structural elements (e.g., walls and doors), building fixtures/furnishings, and one or more interactive target devices (e.g., tintable windows, sensors, emitters, and/or media displays). The digital twin may reside on a server which is accessible via a graphical user interface, or which can be accessed using a virtual reality (VR) user interface. The VR interface may include an augmented reality (AR) aspect. The digital twin may be utilized in connection with monitoring and servicing of the building infrastructure and/or in connection with controlling any interactive target devices, and in providing interactive input (e.g., preferences for one or more environmental settings) from, and feedback to, a customer.

When a new device is installed in the facility (e.g., in a room or space thereof) and is operatively coupled to the network, the new device may be detected (e.g., and included into the digital twin). The detection of the new device and/or inclusion of the new device into the digital twin may be done automatically and/or manually. For example, the detection of the new device and/or inclusion of the new device into the digital twin may be without requiring (e.g., any) manual intervention. Whether present in the original design plans of the enclosure or added at a later time, full details regarding (e.g., each) device (including any unique identification codes) may be stored in the digital twin, network configuration file, interconnect drawing, and/or architectural drawing (e.g., BIM file such as a Revit file) to facilitate the monitoring, servicing, and/or control functions.

In some embodiments, a digital twin comprises a virtual three dimensional (3D) model of the facility. The facility may include static and/or dynamic elements. For example, the static elements may include representations of a structural feature of the facility (e.g., fixtures) and the dynamic elements may include representations of an interactive device with a controllable feature. The 3D model may include visual elements. The visual elements may represent facility fixture(s). The fixture may comprise a wall, a floor, wall, door, shelf, a structural (e.g., walk-in) closet, a fixed lamp, electrical panel, elevator shaft, or a window. The fixtures may be affixed to the structure. The visual elements may represent non-fixture(s). The non-fixtures may comprise a person, a chair, a movable lamp, a table, a sofa, a movable closet, or a media projection. The non-fixtures may comprise mobile elements. The visual elements may represent facility features comprising a floor, wall, door, window, furniture, appliance, people, and/or interactive device(s)). The digital twin may be similar to virtual worlds used in computer gaming and simulations, representing the environment of the real facility. Creation of a 3D model may include the analysis of a Building Information Modeling (BIM) model (e.g., an Autodesk Revit file), e.g., to derive a representation of (e.g., basic) fixed structures and movable items such as doors, windows, and elevators. In some embodiments, the digital twin is defined at least in part by using one or more sensors (e.g., optical, acoustic, pressure, gas velocity, and/or distance measuring sensor(s)), to determine the layout of the real facility. Usage of sensor data can be used (e.g., exclusively) to model the environment of the enclosure. Usage of sensor data can be used in conjunction with a 3D model of the facility (e.g., (BIM model) to model and/or control the environment of the enclosure. The BIM model of the facility may be obtained before, during (e.g., in real time), and/or after the facility has been constructed. The BIM model of the facility can be updated (e.g., manually and/or using the sensor data) during operation and/or commissioning of the facility (e.g., in real time).

In some embodiments, dynamic elements in a digital twin include device settings. The device setting may comprise (e.g., existing and/or predetermined): tint values, temperature settings, and/or light switch settings. The device settings may comprise available actions in media displays. The available actions may comprise menu items or hotspots in displayed content. The digital twin may include virtual representation of the device and/or of movable objects (e.g., chairs or doors), and/or occupants (actual images from a camera or from stored avatars). In some embodiments, the dynamic elements can be devices that are newly plugged into the network, and/or disappear from the network (e.g., due to a malfunction or relocation). The digital twin can reside in any circuitry (e.g., processor) operatively coupled to the network. The circuitry in which the digital circuitry resides may be in the facility, outside of the facility, and/or in the cloud. In some embodiments, a two-way (e.g., bidirectional) link is maintained between the digital twin and a real circuitry. The real circuitry may be part of the control system. The real circuitry may be included in the master controller, network controller, floor controller, local controller, or in any other node in a processing system (e.g., in the facility or outside of the facility). For example, the two-way link can be used by the real circuitry to inform the digital twin of changes in the dynamic and/or static elements so that the 3D representation of the enclosure can be updated, e.g., in real time or at a later (e.g., designated) time. The two-way link may be used by the digital twin to inform the real circuitry of manipulative (e.g., control) actions entered by a user on a mobile circuitry. The mobile circuitry can be a remote controller (e.g., comprising a handheld pointer, manual input buttons, or touchscreen).

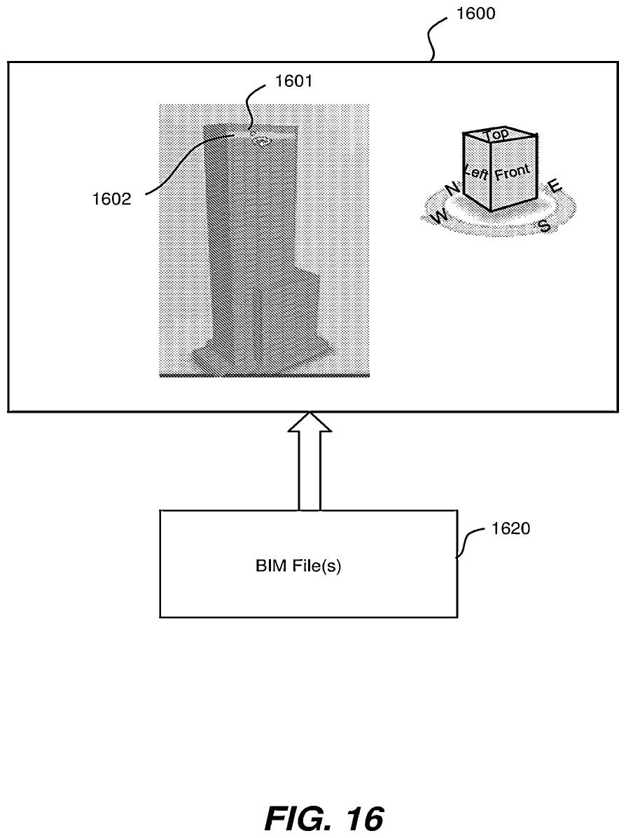

FIG. 16 depicts a user interface 1600 with a visual representation of an example of a digital twin 1601 which may be based, at least in part, on one or more BIM (e.g., Revit) files 1620. In this implementation, digital twin 1601 includes a 3D virtual construct of a facility which may be virtually navigated to view and interact with target devices and/or with different enclosures (e.g., spaces in a building) using an interface device. For example, the user may use the interface device to select and navigate to a floor 1602 of the digital twin 1601 as depicted in FIG. 16. The interface device may be a mobile device (e.g., a smartphone, a laptop, a tablet, a handheld controller, etc.) or another device (e.g., a desktop computer, a wall interface device, etc.). In some embodiments, a virtual representation of the enclosure comprises a virtual augmented reality representation of the digital twin displayed on the mobile device, wherein the virtual augmented reality representation includes virtual representations of at least some of the real target devices and/or spaces in the facility. The navigation within the digital twin using a mobile device may be independent of the actual location of the mobile device, or may coincide with the movement of the mobile device within the real enclosure represented by the digital twin. The mobile device may be operatively (e.g., communicatively) coupled to the network. The mobile device may register its present position in the real facility with a respective position in the digital twin, e.g., using any geo-location technology. For example, the geo-location anchors coupled to the network.

In some embodiments, a mobile device (e.g., a smartphone, tablet, or handheld controller) is utilized to detect commissioning data of respective target devices and transmit the commissioning data to the digital twin and/or BIM system. The mobile device may include geographic tracking capability (e.g., GPS, UWB, Bluetooth, and/or dead-reckoning) so that location coordinates of the mobile device can be transmitted to the digital twin using any suitable network connection established by the user between the mobile device and the digital twin. For example, a network connection may at least partly include the transport links used by a hierarchical controller network within a facility. The network connection may be (e.g., entirely) separate from the controller network of the facility (e.g., using a wireless network such as a cellular network). The target device may be outfitted with an optically recognizable ID tag (e.g., sticker with a barcode or a Quick Response (QR) code). Interaction of the mobile device with the target device may be used to populate a virtual representation of the target device in the digital twin, with a unique identification code and/or other information relating to the target device that is associated with the ID code (e.g., comprised in the ID tag).

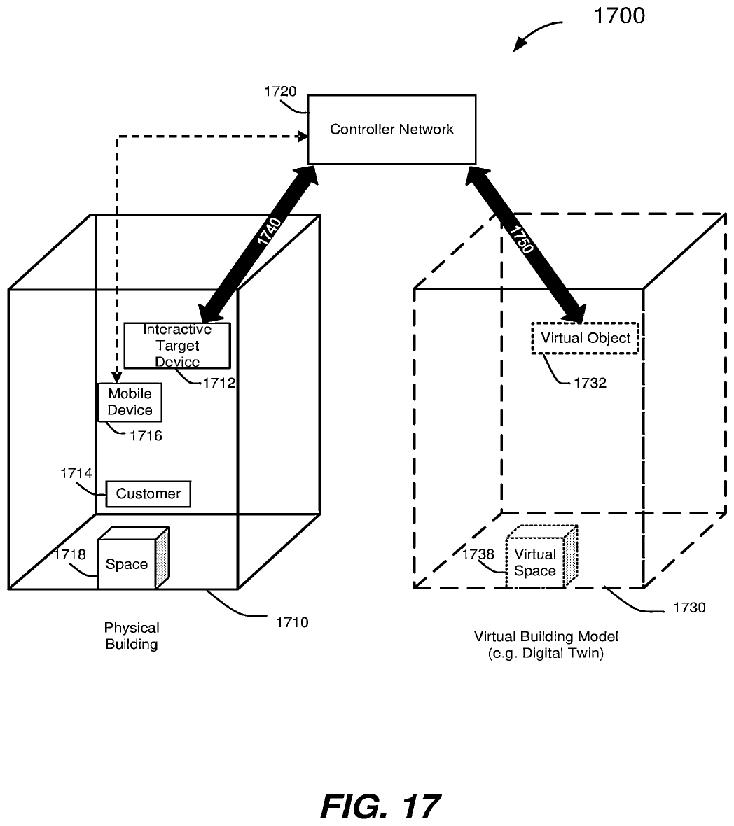

FIG. 17 shows an example embodiment of a control system 1700 includes a controller network 1720 for managing and controlling interactive network devices, e.g., one or more building systems including, for example, one or more tintable windows. The control system 1700 includes a controller network 1720 with one or more controllers such as, for example, one or more of a master controller comprising a processor, a network controller, and local controller. The structure and contents of a real, physical building 1710 are represented in a 3-D model digital twin 1730 as part of a modeling and/or simulation system executed to manage computing assets at the building 1710 and/or control one or more environmental conditions and other conditions at the building 1710. The computing assets may be co-located with or remote from building 1710 and/or the controller network 1720.

In the illustrated example, network link 1740 is connecting controller network 1720 with an interactive target device 1712 (e.g., a tintable window such as an electrochromic window). Interactive target device 1712 is represented as a virtual object 1732 within digital twin 1730. A network link 1740 in building 1710 connects the controller network 1720 with a plurality of network nodes including one or more network interactive target computing devices. In the illustrated example, network link 1740 is connecting controller network 1720 with an interactive target device 1712. Interactive target device 1712 is represented as a virtual object 1732 within digital twin 1730. A network link 1750 connects controller network 1720 with digital twin 1730. In the illustrated example, a customer 1714 is shown located in building 1700 and in communication with a mobile device (e.g., handheld control unit) 1716. Building 1700 includes a physical space 1718 associated with customer 1714. Physical space 1718 in the building 1710 is represented by a virtual space 1738 in digital twin 1730. In certain implementations, physical tintable windows in physical space 1718 are represented by virtual tintable windows in digital twin 1730. Mobile device 1716 may include integrated scanning capability (e.g., a camera for capturing an image of a barcode or QR code), and/or may include, or be in communication with, an identification capture device (e.g., a handheld barcode scanner connected with mobile device 1716, e.g., via a Bluetooth link). ID tags may be comprised of, for example, RFID, UWB, radiogenic, reflective, or absorptive materials to enable use of various scanning tools (e.g., identification capture devices). The code(s) or printed matter on an ID tag may comprise device type, electronic and/or material properties of the target device, serial number, types, identifiers of component parts, manufacturer, manufacturing date, and/or any other pertinent information.

In certain implementations, the digital twin of a facility stored on a server (e.g., server on a cloud network and/or within the facility) may be updated in real-time, or approximately real-time, to include one or more customer preferences such as a condition of an internal space of the facility. In some cases, the digital twin is updated to show real-time or approximately real-time, adjustments to one or more devices (e.g., interactive target device) in the facility. For example, the digital twin may be updated in real-time or approximately real-time, to model future behavior of the facility. For example, a customer may interactively select an internal space in the digital twin to adjust a preferred amount of light in the space. The customer preference of the preferred amount of light is communicated to the server and stored in the digital twin in real-time, or approximately real-time. A visual representation of the future adjusted tint levels of the virtual windows that will meet the preferred condition, and/or the future illuminance levels of the internal spaces in the updated digital twin may be provided in real time, or approximately real-time, to the customer to show future behavior of the facility.

Some examples of digital twins, user interfaces, commissioning, networks, smart objects, 3D representations of buildings and spaces, zones of tintable windows and other groupings of tintable windows, and controlling tintable windows are described in U.S. patent application Ser. No. 17,400,596, titled “AUTOMATED COMMISSIONING OF CONTROLLERS IN A WINDOW NETWORK,” filed on Aug. 12, 2021, which is hereby incorporated by reference in its entirety.

### II. Building Wellness Index and Composite Indices

### Building Wellness Index

Certain embodiments pertain to systems and methods that can be used to obtain and process data for a building for the purpose of calculating a wellness index for the building (i.e. a building wellness index (BWI)). The data can be or include one or more parameters related to, for example, air and water quality, occupancy, body temperature, reported illnesses, and/or previous building maintenance or cleaning. In general, a BWI may provide an overall measure or indication of a wellness of the building. The BWI can indicate, for example, how risky it may be from a personal wellness standpoint for a person to enter or spend time in the building. For example, the BWI can indicate a likelihood that the building may be contaminated with a virus (e.g., a coronavirus) or other pathogen. Additionally, or alternatively, the BWI can provide an indication of how likely it may be that a person who enters or spends time in the building will be exposed to a virus or other pathogen.

In some implementations, the BWI and/or supporting data may be made available to one or more occupants of the building such as tenants or people who otherwise visit or enter the building (e.g., vendors, maintenance staff, etc.). Such information can be made available through a software application (e.g., installed on user mobile phones, personal computers, etc.), digital signage, text messaging or notifications, a tenant interface such as a digital twin, and/or building manager tools. Availability of the building wellness index and related data can empower occupants and building staff to make data-driven decisions around managing staff and resources in the context of any wellness risks associated with conditions in the building. For example, the BWI can be used to facilitate a corrective action to improve the current BWI. Such action can be or include, for example, requiring people to vacate the building or move to specific portions of the building, requiring people to reduce occupancy in the building, requiring use of personal protective equipment, and/or performing maintenance or cleaning on one or more contaminated or damaged building components or areas.

In various examples, the parameters used to calculate the BWI may include data related to a condition of the building and/or the building's occupants such as tenants and/or visitors. The parameters can include, for example, building occupancy data (e.g., a number of occupants and/or a population density for the building), occupant wellness report data (e.g., data indicating one or more occupants is presently sick or recovering from recent illness), air quality data, water quality data (e.g., data describing water quality for a cooling tower), building cleanliness data (e.g., a length of time since a previous deep cleaning or recent pathogen exposure), occupant body temperature data, historical building wellness index data (e.g., a rate of change or trend for the building wellness index), or any combination thereof. Such data can be collected or obtained from one or more building managers, occupants, medical professionals, cleaning professionals, other personnel, or measurement devices.

In certain embodiments, a building wellness index (BWI) may be calculated by combining the values of one or more of the parameters (also referred to herein as “metrics”). For example, each parameter can be assigned a numerical value in a range (e.g., from 0 to 1, or from −1 to 1) that might indicate a wellness risk associated with the parameter. For example, if a parameter is evaluated and indicates a high risk of being detrimental to wellness, the scoring of the parameter may at the high end of the range (e.g., to 0 or −1). If the parameter is evaluated and indicates a low risk of being detrimental to wellness risk, the scoring of the parameter may be set to the low end of the range (e.g. to 1). In another example, a parameter evaluated to be at high risk to wellness may be assigned a value at the low end of the range and a parameter evaluated to be at low risk to wellness may be assigned a value at the high end of the range.

Each parameter can be assigned a weight, and the BWI can be calculated by combining the weighted parameters as follows:

$$Building Wellness Index ( BWI ) = {\sum }_{1}^{N} {W}_{i} {P}_{i} = {W}_{1} {P}_{1} + {W}_{2} {P}_{2} + … + {W}_{N} {P}_{N} \qquad ( Eqn . 1 )$$

where: Pᵢ refers to one or more parameters, Wᵢ refers to one or more corresponding weighting factors, and N is the total number of parameters and corresponding weighting factors.

Other methods for calculating a BWI are contemplated. For example, one or more machine learning models or classifiers can be trained and used to calculate the BWI. For example, one or more parameters can be provided to a machine learning model as input, and the BWI can be provided by the machine learning model as output. The machine learning model can be trained to identify building wellness issues using training data that includes, for example, parameters and corresponding values for the BWI. Additionally, or alternatively, one or more functional forms can be used to combine the parameters (e.g., besides the linear form in Eqn. 1) and calculate the BWI. Such functional forms can be or include, for example, non-linear functions, exponential functions, logarithmic functions, quadratic functions, and the like.

Advantageously, the systems and methods described herein can improve accuracy and/or automation of data processing. Data related to a BWI may be collected from a variety of sources, including sensors in and around buildings (e.g., body temperature scanners, air quality sensors, security cameras, occupancy sensors, social distancing badges, etc.), push buttons, medical testing labs, and/or information provided by occupants (e.g., through surveys, work orders, or self-reporting). In some cases, the systems and methods can aggregate such data in an automated manner to calculate a BWI and take corrective action, as needed. Compared to other approaches that rely on manual data collection and analysis, some computer-implemented systems, connected sensors, algorithms, and machine learning techniques described herein may achieve a more automated approach for processing data related to building wellness that may help ensure issues related to building wellness are identified and addressed in an efficient and accurate manner.

Certain aspects pertain to computer-implemented methods of managing building wellness. In some embodiments, the method includes the steps of: obtaining parameters for a building (e.g., an office building) having an occupant(s) (e.g., an employee(s)); processing the parameters to determine a current BWI for the building; and, based on the current BWI, sending a message regarding the current BWI to a recipient(s) (e.g., a building occupant), (ii) displaying the current BWI for a user(s), and/or identifying a remediation action(s) to improve the current building wellness index. In some variations, the parameters may include building occupancy data, occupant wellness report data, air quality data, water quality data, building cleanliness data, occupant body temperature data, historical building wellness index data, and/or any combination thereof. In these cases, the current BWI may provide an indication of a risk of being exposed to a pathogen (e.g., a virus) inside the building.

In some implementations, the method may use data including one or more of the following: (i) building occupancy data may include a number of occupants for the building and/or a population density for the building, (ii) occupant wellness report data may include data indicating an occupant(s) is presently sick or recovering from recent illness, (iii) water quality data may include data describing water quality for a cooling tower, (iv) building cleanliness data may include a length of time since a previous deep cleaning or recent pathogen exposure (v) and/or historical building wellness index data comprising at least one of a rate of change for the BWI or a trend for the BWI.

In some applications, displaying the current BWI may include presenting the current BWI on a client device of a user(s) and/or identifying a remediation action(s) may include instructing people to vacate the building, move to a specific portion of the building, use personal protective equipment inside the building, and/or clean an area(s) of the building.

Certain aspects pertain to systems for managing building wellness. In some embodiments, system includes a computer processor(s) adapted to perform operations. In some embodiments, stored instructions in the computer processor(s) include: obtaining parameters for a building (e.g., an office building) having an occupant(s) (e.g., an employee(s)); processing the parameters to determine a current BWI for the building; and, based on the current BWI, sending a message including the current BWI to a recipient(s) (e.g., an occupant(s), displaying the current BWI for a user(s), and/or identifying a remediation action(s) to improve the current BWI. In some variations, the parameters may include building occupancy data, occupant wellness report data, air quality data, water quality data, building cleanliness data, occupant body temperature data, historical building wellness index data, and/or any combination thereof. The current BWI provides an indication of a risk of being exposed to a pathogen (e.g., a virus) inside the building.

In some implementations, building occupancy data may include a number of occupants for the building and/or a population density for the building; occupant wellness report data may include data indicating an occupant(s) is presently sick or recovering from recent illness; water quality data may include data describing water quality for a cooling tower; building cleanliness data may include a length of time since a previous deep cleaning or recent pathogen exposure; historical building wellness index data may include a rate of change for the BWI and/or a trend for the BWI.

In some applications, displaying the current BWI may include presenting the current BWI on a client device of a user(s) and/or identifying the remediation action(s) may include instructing people to vacate the building, move to a specific portion of the building, use personal protective equipment inside the building, and/or clean an area(s) of the building.

Certain aspects pertain to non-transitory computer-readable medium having instructions stored thereon that, when executed by a computer processor(s), cause the computer processor(s) to perform operations. In some embodiments, the stored instructions include: obtaining parameters for a building (e.g., an office building) having an occupant(s) (e.g., an employee(s)); processing the parameters to determine a current BWI for the building; and, based on the current BWI, sending a message including the current BWI to a recipient(s) (e.g., an occupant(s), displaying the current BWI for a user(s), or identifying a remediation action(s) to improve the current BWI. In some variations, the parameters may include building occupancy data, occupant wellness report data, air quality data, water quality data, building cleanliness data, occupant body temperature data, historical building wellness index data, and/or any combination thereof. The current BWI provides an indication of a risk of being exposed to a pathogen (e.g., a virus) inside the building.

Advantageously, systems, methods, and apparatus of certain embodiments described herein may be structured and arranged to calculate a building wellness index (BWI) and categorize the BWI score at one of a plurality wellness levels. For example, a BWI score may be assigned one of four wellness levels: “good,” “moderate,” “use caution,” and “alert.” Those of ordinary skill in the art can appreciate that the number and names of wellness levels may vary by implementation and that the following description is meant to be instructive and illustrative of an example of a BWI scoring technique. In calculating a BWI, assumptions and considerations may include the desire to avoid (i) recommendations that violate any lease and (ii) claims that may directly impact personal and/or individual health decisions. Moreover, the BWI calculated may be (i) based at least in part on governmental guidelines when determining any occupancy thresholds and (ii) based at least in part on established (e.g., ASHRAE, CDC, EPA, and the like) baselines for any thresholds relating to health and wellness. Furthermore, although all data will be available for use in calculating the BWI, not all data may factor into the risk level calculation.

For example, when the data used to calculate a BWI results in a “good” level, there may be deemed no increased health risk to occupants (e.g., tenants, building employees, building visitors, and so forth); hence, occupants may be free to enter into and work freely within the building without the need for wearing increased personal protection equipment (PPE), social distancing, or other restrictive practices. Alternatively, when the data used to calculate a BWI results in an “alert” level, conditions within the building may be deemed life threatening or, in the alternative, the building may need to comply with a government “shelter in place” order. Under “alert” level conditions, potential occupants (e.g., tenants, building employees, building visitors, and so forth) may avoid coming to the office building and work from home. The intermediate alert levels (“moderate” and “use caution”) stand somewhere between the ideal conditions of “good” and the heightened risk conditions of “alert.” Thus, the “moderate” and “use caution” levels reflect a decrease in life threatening conditions and/or government restrictions, resulting in a corresponding decrease in usage limitations and required safety practices for occupants.

In calculating a BWI rating, direct, indirect, and other parameter (also referred to herein as metrics) may be taken into account. As previously stated, although all data will be available for use in calculating the BWI, not all data may factor into the calculation of the BWI and assigning of risk level. Direct parameters may include direct indicators of a possible risk, e.g., due to a pathogen (e.g., a virus, such as COVID-19), and are primary contributors to risk level escalation. Indirect parameters may indicate overall risk trends or otherwise contribute to possible risk, e.g., due to a pathogen (e.g., a virus, such as COVID-19), and a secondary contributor(s) to risk level escalation. Other parameters that are neither direct nor indirect may have no correlation to possible risk due to a pathogen (e.g., a virus, such as COVID-19); however, they are important to overall wellness and health of building occupants.

Table I provides exemplary risk rate criteria using direct parameters for each of four wellness levels “good,” “moderate,” “use caution,” and “alert.” In some implementations, these direct parameters may include, e.g., one or more of historic risk, building density, pathogen (e.g., COVID) space testing, reported pathogen (e.g., COVID) cases, or elevated employee temperatures.

TABLE I
LEVEL CRITERIA (Direct Parameters)

| | Level | | | |
|---|---|---|---|---|
| | ALERT | USE CAUTION | MODERATE | GOOD |
| | Parameter | | | |
| | IF ANY OF THE | IF ANY OF THE | IF ANY OF THE | IF ALL OF THE |
| | FOLLOWING ARE | FOLLOWING ARE | FOLLOWING ARE | FOLLOWING ARE |
| | TRUE | TRUE | TRUE | TRUE |
| Historic Risk | N/A | 24 hours < since | 24 hours < since | >24 hours since |
| | | “ALERT” status | “USE CAUTION” | “MODERATE” status |
| | | | status | |
| Building | “Shelter in place” | Building density | Building density | No government |
| Density | or “stay at home” | <110% of t get and | >110% of t get and | agency |
| | order from CDC | government agency | government agency | recommendation in |
| | or government | recommendation in | recommendation in | effect |
| | agency | effect | effect | |
| Pathogen | | 3 samples < detected | Between 1 and 3 | More than one week |
| (COVID) | | in the last week | samples detected in | since last sample was |
| Space Testing | | | the last week | discovered. |
| Reported | | 1% of building | 1 or more cases of | No cases of highly |
| Pathogen | | population < | pathogen reported in | infectious diseases |
| (COVID) | | reported with | the last two (2) | reported in the last |
| Cases | | pathogen in last | weeks | two weeks |
| | | two (2) weeks | | |
| Elevated | | 1 standard deviation < | | Monthly average |
| Employee | | above monthly | | baseline of elevated |
| Temperatures | | average baseline | | temperature |
| | | | | employee |

Table II provides exemplary risk rate criteria due to other parameters for each of four BWI levels “good,” “moderate,” “use caution,” and “alert.” In some implementations, these parameters may include, for example, one or more of carbon monoxide (CO) levels, levels of particulate matter (PM₁₀), ozone levels, volatile organic compounds (VOC) levels, formaldehyde levels, or legionella levels (for buildings that draw potable water from cooling water towers). The other parameters in Table II pertain more to the environment and how it may affect human beings as opposed to parameters that directly affect the building itself.

TABLE II
LEVEL CRITERIA (Other Parameters)

| | Level | | | | | |
|---|---|---|---|---|---|---|
| | ALERT | | USE CAUTION | MODERATE | GOOD | |
| | Parameter | | | | | |
| | IF ANY OF THE | | IF ANY THREE (3) | IF ANY THREE (3) | | |
| | FOLLOWING | | OF THE FOLLOWING | OF THE FOLLOWING | | |
| | ARE TRUE | | ARE TRUE | ARE TRUE | | |
| CO | ≥12.4 | ppm | >9.5 and | >4.4 ppm and | ≤4.4 | ppm |
| (in 8 hours) | | | ≤12.4 ppm | ≤9.4 ppm | | |
| PM10 | >255 | μg/m³ | >155 μg/m³ and | >54 μg/m³ and | ≤54 | μg/m³ |
| (in 24 hours) | | | ≤254 μg/m³ | ≤154 μg/m³ | | |
| OZONE | >0.086 ppm and | | >0.070 ppm and | >0.054 ppm and | ≤0.054 | ppm |
| (in 8 hours) | ≤0.105 ppm | | ≤0.085 ppm | ≤0.070 ppm | | |
| VOC | >1000 μg/m3 | | ≥500 μg/m³ and | >0.027 ppm | ≤500 | μg/m3 |
| Formaldehyde | ≥5 | ppm | <1000 μg/m³ | | | |

(in 8 hours)

| | | |
|---|---|---|
| Legionella | YES | NO |

Table III provides exemplary risk rate criteria due to indirect factors for each of the four levels “good,” “moderate,” “use caution,” and “alert.” These indirect factors may include, for example, one or more of carbon dioxide (CO₂) levels, humidity, levels of particulate matter (PM2-5), or employee absenteeism. The indirect factors in Table III pertain to the environment and how it may affect human beings.

TABLE III
LEVEL CRITERIA (Indirect Parameters)

| | Level | | | |
|---|---|---|---|---|
| | ALERT | USE CAUTION | MODERATE | GOOD |
| | Parameter | | | |
| | | IF ANY OF THE | IF ANY OF THE | IF ANY OF THE |
| | IF ANY OF THE | FOLLOWING | FOLLOWING | FOLLOWING |
| | FOLLOWING | ARE TRUE | ARE TRUE | ARE TRUE |
| CO₂ | | | >10% above | ≤10% below |
| | | | (ambient) outside | (ambient) outside |
| | | | air CO₂ levels | air CO₂ levels |
| Humidity | | | <40% or >60% | ≥40% and ≤60% |
| PM2-5 | >55.4 μg/m³ | ≥35.4 μg/m³ | | ≤35.4 μg/m³ |
| (in 24 hours) | | and ≤55.4 μg/m³ | | |
| Employee | | | Illness-related | Illness-related |
| Absenteeism | | | Employee | Employee |
| | | | absenteeism has | absenteeism |
| | | | trended upward for | has remained |
| | | | 3 days | stable or decreased |
| | | | | for at least 3 days |

### B. Examples of System Architecture and Computing System

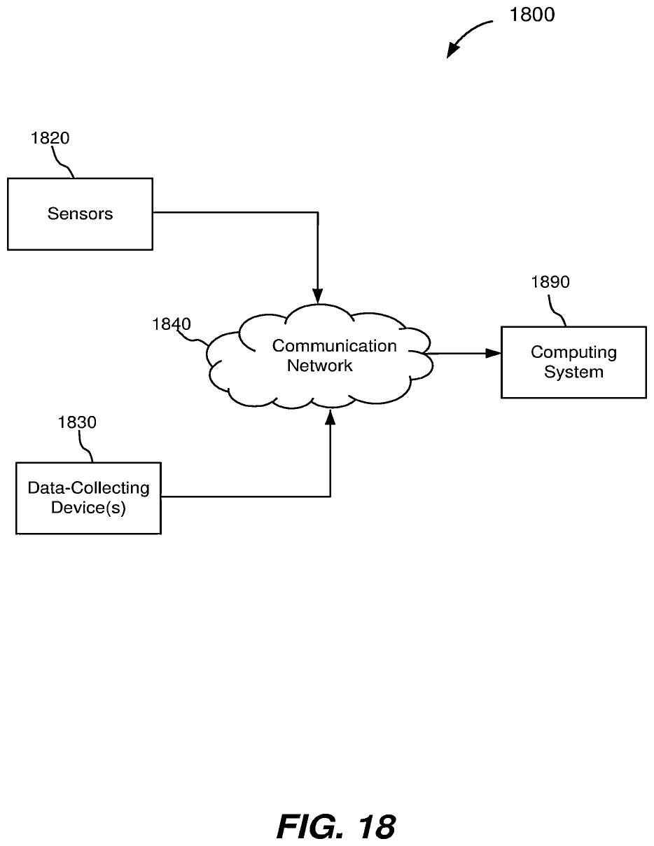

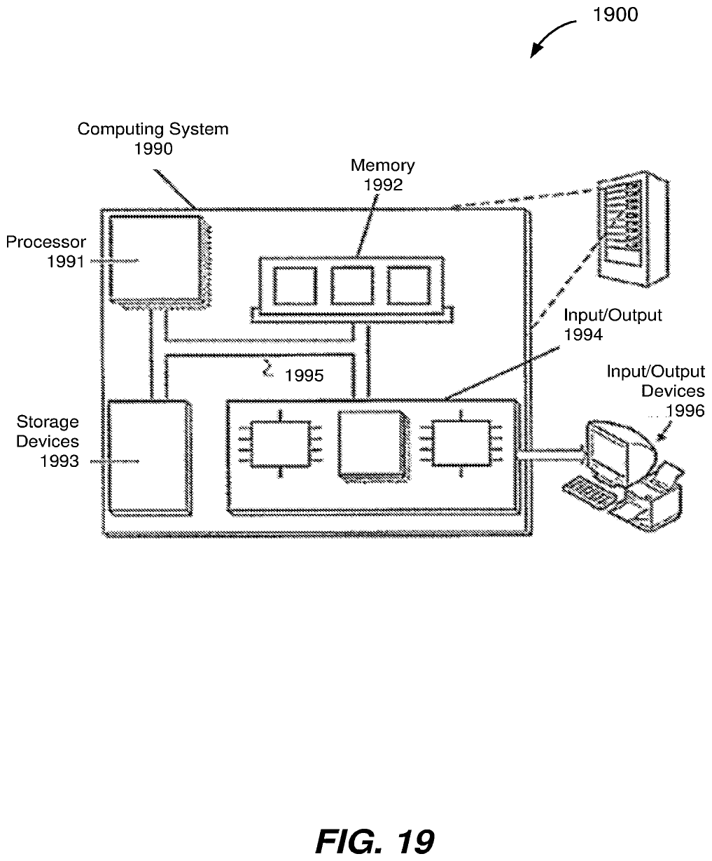

FIG. 18 is a schematic drawing depicting an exemplary architecture of an embodiment of a system 1800 for managing building wellness and/or one or more building system. System 1800 includes a plurality of sensors 1820 (e.g. a sensor ensemble) and/or one or more data-collecting devices 1830, as well as a computing system 1890. Computing system 1890 is configured to calculate a building wellness index and/or one or more composite indices using, inter alia, sensor data from plurality of sensors 1820 and/or other data from data-collecting device(s) 1830. The data-collecting devices may collect and process data including, for example, sensor data from the plurality of sensors 1820, data from one or more surveys, questionnaires, or work orders, data from user input such as operator (e.g. occupant) input, and so forth.

System 1800 also includes a communication network 1840 that enables the transfer of (e.g., communication and/or power) signals and data between computer-based system 1890 and sensors 1820 and data-collecting device(s) 1830, such that data collected by the plurality of sensors 1820 and data-collecting device(s) 1830 may used to evaluate what is occurring, what may be occurring, and/or predict what is likely to occur within the (e.g., office) building to evaluate the contributing metrics and calculate a business wellness index and/or one or more composite business wellness indices. Moreover, the evaluation of the data and insights may be used to determine one or more remediation, preventive, and/or other actions that may be taken to improve the quality of life for one or more occupants within the building. In some implementations, such actions may be communicated (e.g., via email, text message, phone call, digital twin, and the like) to the occupants and/or to other individuals or building departments that may be responsible for effecting the one or more actions.

In some embodiments, computing system 1890 includes stored instructions for performing one or more operations that may include, for example, obtaining and processing data that can be used to determine values of contributing parameters for an enclosure (e.g., a building, a room such as an office in a building, etc.) having one or more occupants (e.g., tenant employees and/or visitors).

Some examples of data used to determine the values of parameters may include, for the purpose of illustration rather than limitation: building occupancy data, occupant wellness report data, air quality data, water quality data, building cleanliness data, occupant body temperature data, historical building wellness index data, and/or any combination thereof. Building occupancy data may include a current number of occupants for an enclosure such as a building or a space in a building. The current number of occupants may be, for example, a floor-by-floor and a room-by-room assessment and/or a population density for a building. Occupant wellness report data may include data, for example, indicating that one or more occupants may be presently sick or recovering from recent illness. Water quality data may include data, for example, describing water quality for a cooling tower. Building cleanliness data may include, e.g., a duration of time since a previous cleaning or recent pathogen exposure. Historical building wellness index data may include, e.g., a rate of change for the building wellness index and/or a trend for the building wellness index.

Computing system 1890 may also include stored instructions for calculating the scores of the parameters, determining their weighting factors, and calculating a current building wellness index (BWI) for a building and/or one or more composite indices for a space of the building. In some cases, the calculated BWI or a composite index may be an indicator of a potential risk to an occupant, e.g., of exposure to a pathogen (e.g., a virus, COVID-19, and so forth). In one implementation, if the calculated BWI or composite index is indicative of a potential risk based on the calculated BWI or composite wellness index, a message or other notification may be sent to the occupant and/or an individual that may be designated, of otherwise have the capability, to take some action to address the potential risk and/or improve the current BWI or composite index. For example, a message or other notification may be sent to a current occupant(s) and/or one or more other individuals with the current BWI or current composite index) displaying the current BWI or composite index to a user(s), and/or identifying a remediation action(s) to improve the current BWI or composite index. The current BWI and/or current composite index(es) may be displayed, for example, on a digital twin of the building or other user interface. The current BWI or composite index may provide an indication of a potential or actual risk, e.g. of exposure to a pathogen (e.g., a virus) inside the building. In one example, displaying the current BWI or composite index may include presenting the current BWI or composite index on a client device of a user(s), while identifying the one or more remediation action(s) may include, e.g., instructing occupant(s) to vacate the building, move to a specific portion of the building, use personal protective equipment inside the building, and/or clean or otherwise perform one or more remediation actions on one or more areas of the building.

In some cases, for the purpose of determining building occupancy (e.g., occupancy on a floor-by-floor and/or room-by-room basis), building density, building foot traffic, tenant usage, and tenant engagement, one or more sensors 1820 and/or data-collecting device(s) 1830 may include one or more threshold counters (e.g., counters at points of access and egress from the building or another enclosure) for counting and recording the number of building occupants (e.g., tenants and/or visitors) that have entered/exited an enclosure such as the building or an office or other space in the building, closed-circuit television (CCTV) for identifying discrete building occupants who have entered/exited the enclosure, and/or individual access badges that may be scanned automatically or manually when the occupant (e.g., tenant(s) or visitor(s)) enters/exits the enclosure. In addition to, for example, predicting future occupancy, ascertaining foot traffic trends, and managing elevator queuing, such data may be used, inter alia, to ensure that the number of personnel within the enclosure does not exceed government (e.g., health and safety) guidelines and/or protocols.

In some cases, data gathered by the one or more sensors 1820 and/or data-collecting device(s) 1830 may be used to estimate optimal cleaning scheduling and staffing so that janitorial and cleaning staff operations may be adjusted. For example, under “Use Caution”, “Moderate,” and/or “Good” levels, janitorial and custodial staff operations may be adjusted to continuously clean all high touch areas and high touch points, for example, in the building lobby and common areas. For the purpose of illustration rather than limitation, high touch points may include, for example, door handles, turnstiles, lobby desks, elevator buttons, sneeze guards, revolving doors, and the like. Furthermore, when a calculated BWI or composite index indicates a condition regarding public health and safety that indicates an elevated risk (e.g., “Use Caution” level), janitorial and custodial personnel may be directed to perform additional cleaning, targeting paths of occupant travel and common areas in line with CDC guidelines. Sensors 1820 and data-collecting device(s) 1830 installed in building restrooms may also include push buttons by which users of the facilitates may indicate facility use, so that restocking of restroom supplies and periodic cleaning may be tailored to such use. Sensors 1820 and data-collecting device(s) 1830 may also be installed at other building amenity centers (e.g., snack bar, cafeteria, and so forth) to provide data regarding amenity usage from which cleaning schedules may be determined.

For the purpose of determining workspace needs and trends as may be used in evaluating metrics, sensors 1820 and data-collecting device(s) 1830 may include (e.g., floor and/or room) one or more occupancy sensors. Occupancy sensors may be employed to take sensor data that can be used to determine office assignment and meeting space needs and utilization, employee space needs, employee work habits, team collaboration, employee interaction, and the like. Sensor data from amenity occupancy sensors may be used to evaluate amenity needs and utilization.

For the purpose of evaluating metrics associated with building and/or occupant wellness and/or occupant comfort, sensors 1820 and data-collecting device(s) 1830 may include (e.g., indoor and/or outdoor) one or more air quality (AQ) sensors, one or more temperature sensors (e.g., non-invasive elevated body temperature sensors), and the like. AQ sensors may include one or more humidity sensors, which may be used, inter alia, to maintain a humidity level within an enclosure (e.g., a building) that may, e.g., suppress pathogen transmission. For example, non-invasive, high-occupancy body temperature scanners may be installed in a building lobby and all occupants (e.g., tenants and/or visitors) may be required to pass through the scanners to determine. In addition to identifying individual occupants whose health may jeopardize that of other building occupants, such data may be used, for example, to determine when to replace and/or recalibrate AQ sensors, when to mitigate AQ events, and so forth. As another example, social distancing badges and/or sensors may also be used to track contact and/or distance between occupants.

In some implementations, a medical screening or care site and/or a medical testing lab may be in a building. In addition to wearing facial masks in accordance with CDC guidelines in all common areas, occupants (tenants and/or visitors) may be required to complete a (e.g., COVID-related) building access questionnaire before accessing the building at, for example, the medical screening or care site. Common areas may include, for example, lobbies, elevators, stairwells, bathrooms, amenity centers, and so forth. Facial masks, protective gloves, hand sanitizer, and the like may also be provided at the medical screening or care site. In some instances, pathogen (e.g., COVID) testing may be performed and/or vaccinations may be provided at, e.g., the medical screening or care site and/or the medical testing lab.

In some implementations, occupants may be provided with a tenant application. In some embodiments, the tenant application is a mobile application (“mobile app”) used by occupants of the building to perform daily activities, including completion of a health attestation. The tenant application also provides mechanisms to publish surveys to the occupants to get their feedback on conditions within the building, such as overall cleanliness. Data from the tenant application, as well as health attestation, surveys, or other employee activity, may be used as inputs for methods described herein.

In some cases, publicly-available data may also be used to determine building wellness. Some examples of publicly-available data includes current data regarding local hospitalization rates for flu-like symptoms, COVID cases in a geographical region, and/or public mobility data that can be used to ascertain local crowding and density.

Sensors 1820 and data-collecting device(s) 1830 may also include one or more sensors that are included in building systems, for example, for monitoring some aspect of a building or other enclosure. Among these building system sensors 1820 and data-collecting device(s) 1830 are utility meters, water and air temperature sensors, furnace or boiler sensors, building work orders, and the like. Data from these building system sensors may be uses to evaluate energy usage and energy cost for the purpose of monitoring energy performance of an enclosure. Such data may also be used to provide an indication of general building and/or building plant preventive maintenance needs that may be addressed, e.g., prior to an emergency repair. In addition or alternatively, sensors 1820 and data-collecting device(s) 1830 may include one or more sensor ensembles with a diverse group of sensors or similar sensors.

FIG. 19 shows a block diagram of an exemplary system 1900 with a computing system 1990 that may be used to implement certain aspects of technology described herein. General-purpose computers, network appliances, mobile devices, or other electronic systems may also include at least portions of computing system 1990. In some implementations, computing system 1990 may include a processor 1991, a memory 1992, a storage device 1993, and an input/output device 1994. Each of the components 1991, 1992, 1993, and 1994 may be interconnected, for example, using a system bus 1995.

Advantageously, the (e.g., single- or multi-threaded) processor 1991 is capable of processing instructions for execution within computing system 1990. In some variations, these instructions may be stored in memory 1992 and/or on storage device 1993.

Memory 1992 stores information within computing system 1990. In some implementations, memory 1992 may be a non-transitory computer-readable medium. In some implementations, memory 1992 may be a volatile memory unit. In some implementations, memory 1992 may be a non-volatile memory unit.

Storage device 1993 is capable of providing mass (e.g., data) storage for computing system 1990. In some implementations, storage device 1993 may be a non-transitory computer-readable medium. In various different implementations, storage device 1993 may include, for example, a hard disk device, an optical disk device, a solid-date drive, a flash drive, and/or some other large capacity storage device. For example, storage device 1993 may store long-term data (e.g., database data, file system data, etc.).

In some embodiments, input/output device 1994 performs input/output operations for computing system 1990. For example, in some implementations, input/output device 1994 may include one or more of: a network interface device (e.g., an Ethernet card), a serial communication device, (e.g., an RS-232 port), a wireless interface device (e.g., an 802.11 card), a 3G wireless modem, and/or a 4G wireless modem. In some implementations, input/output device 1994 may include driver devices configured to receive input data and to send output data to other input/output devices 1996, e.g., keyboard, printer, and display devices. In some examples, mobile computing devices, mobile communication devices, and other devices may be used.

In some implementations, at least a portion of the approaches described above may be realized by instructions that, upon execution, cause one or more processing devices to carry out the processes and functions described above. Such instructions may include, for example, interpreted instructions such as script instructions, or executable code, or other instructions stored in a non-transitory computer readable medium. Storage device 1993 may be implemented in a distributed way over a network, for example as a server farm or a set of widely distributed servers, or may be implemented in a single computing device.

Although an exemplary processing system 1990 has been described in FIG. 19, embodiments of the subject matter, functional operations and processes described in this specification can be implemented in other types of digital electronic circuitry, in tangibly-embodied computer software or firmware, in computer hardware, including the structures disclosed in this specification and their structural equivalents, or in combinations of one or more of them. Embodiments of the subject matter described in this specification can be implemented as one or more computer programs, i.e., one or more modules of computer program instructions encoded on a tangible nonvolatile program carrier for execution by, or to control the operation of, data processing apparatus. Alternatively, or in addition, the program instructions can be encoded on an artificially generated propagated signal, e.g., a machine-generated electrical, optical, or electromagnetic signal that is generated to encode information for transmission to suitable receiver apparatus for execution by a data processing apparatus. The computer storage medium can be a machine-readable storage device, a machine-readable storage substrate, a random or serial access memory device, or a combination of one or more of them.

### C. Composite Indices

Certain embodiments pertain to methods and systems that can dynamically evaluate the safety, health, and/or operational efficiency of an enclosure (e.g., a building or a space in a building) based on data from, for example, environmental sensors, occupancy counters, building access data, surveys, work orders, and/or utilities monitoring. In certain implementations, one or more composite indices may be calculated for the enclosure. For example, a plurality of three composite indices may be calculated: a health index, a safety index, and a performance index. A safety index may provide a measure of whether the enclosure is considered safe for its current occupant(s) from hazards such as, e.g., physical hazards, security risks and/or infectious disease transmission. A health index may provide a measure of whether the enclosure is generally healthy, comfortable, and productive for its current occupants. A performance index may provide a measure of whether the enclosure is operationally performing sustainably from, e.g., an energy, water, and waste perspective, and/or whether one or more building systems at the location of the enclosure are operating properly.

Each composite index may be associated with one or more subgroups of contributing metrics. The score of each subgroup of contributing metrics may be referred to as a subgroup index or a subgroup score. A composite index may be calculated by combining weighted scores of the subgroups, as follows:

$$Composite Index ( C I ) = {\sum }_{j = 1}^{m} {GI}_{j} {GW}_{j} \qquad ( Eqn . 2 )$$

where m (e.g., 1, 2, 3, etc.) refers to the number of subgroups associated with the composite index, GIⱼ refers to a subgroup index, and GWⱼ refers to a subgroup weighting factor corresponding to the subgroup index GIⱼ.

In certain implementations, a plurality of composite indices may be calculated according to the following:

$$Composite {Indices ( CI )}_{i} = {\sum }_{j = 1}^{m} {GI}_{i , j} {GW}_{i , j} \qquad ( Eqn . 3 )$$

where i=1 to p, p (e.g., 2, 3, etc.) refers to the number of composite indices calculated, m (e.g., 1, 2, 3, etc.) refers to the number of subgroups of contributing metrics associated with the composite index, GI_{i,j} refers to the j^{th} subgroup index associated with the i^{th} composite index, and GW_{i,j}, refers to the subgroup weighting factor.

Each subgroup index may be calculated by combining weighted scores of contributing metrics associated with the subgroup, as follows:

$$Subgroup Index ( {GI}_{i , j} ) = {\sum }_{k = 1}^{n} {P}_{i , j , k} {W}_{i , j , k} \qquad ( Eqn . 4 )$$

where n (e.g., 1, 2, 3, etc.) refers to the number of contributing metrics in the subgroup, j refers to the subgroup, and k refers to the composite index, and P_{i,j,k} refers to the k^{th} contributing metric associated with the j^{th} subgroup and i^{th} composite index, and W_{i,j,k} refers to the corresponding weighting factor for the contributing metric (sometimes referred to herein as “sub-weight”).

In one implementation, three composite indices may be calculated for an enclosure: a safety index, a health index, and a performance index, according to the following equations:

$$Safety Index = {\sum }_{j = 1}^{q} {GI}_{1 , j} {GW}_{1 , j} \qquad ( Eqn . 5 a )$$

where: j=1 to q and q (e.g., 1, 2, 3, etc.) refers to the number of subgroups of contributing metrics associated with the composite safety index.

$$Health Index = {\sum }_{j = 1}^{r} {GI}_{2 , j} {GW}_{2 , j} \qquad ( Eqn . 5 b )$$

where: j=1 tor and r (e.g., 1, 2, 3, etc.) refers to the number of subgroups of contributing metrics associated with the composite health index.

$$Performance Index = {\sum }_{j = 1}^{s} {GI}_{3 , j} {GW}_{3 , j} \qquad ( Eqn . 5 c )$$

where: j=1 to s and s (e.g., 1, 2, 3, etc.) refers to the number of subgroups of contributing metrics associated with the composite performance index.

Each subgroup index for the safety index, (GW_{1,j}), in Eqn. 5a, may be calculated as follows:

$$Subgroup Index for Safety Index ( {GW}_{1 , j} ) = {\sum }_{k = 1}^{n} {P}_{1 , j , k} {W}_{1 , j , k} \qquad ( Eqn . 6 a )$$

where k=1 to n, n (e.g., 1, 2, 3, etc.) refers to the number of contributing metrics in the j^{th} subgroup, P_{1,j,k} refers to the k^{th} contributing metric associated with the j^{th} subgroup of the safety index, and W_{1,j,k} refers to the corresponding weighting factor for the contributing metric.

Each subgroup index for the health index, (GW_{2,j}), in Eqn. 5b, may be calculated as follows:

$$Subgroup Index for Health Index ( {GW}_{2 , j} ) = {\sum }_{k = 1}^{n} {P}_{2 , j , k} {W}_{2 , j , k} \qquad ( Eqn . 6 b )$$

where k=1 to n, n (e.g., 1, 2, 3, etc.) refers to the number of contributing metrics in the j^{th} subgroup, P_{2,j,k} refers to the k^{th} contributing metric associated with the j^{th} subgroup of the health index, and W_{2,j,k} refers to the corresponding weighting factor for the contributing metric.

Each subgroup index for the performance index, (GW_{3,j}), in Eqn. 5b, may be calculated as follows:

$$Subgroup Index for Performance Index ( {GW}_{3 , j} ) = {\sum }_{k = 1}^{n} {P}_{3 , j , k} {W}_{3 , j , k} \qquad ( Eqn . 6 c )$$

where k=1 to n, n (e.g., 1, 2, 3, etc.) refers to the number of contributing metrics in the j^{th} subgroup, P_{3,j,k} refers to the k^{th} contributing metric associated with the j^{th} subgroup of the performance index, and W_{3,j,k} refers to the corresponding weighting factor for the contributing metric.

Composite indices may be based on data for contributing metrics from various data sources including, for example, sensor data from one or more sensors (e.g., a sensor ensemble), data from one or more surveys, questionnaires, and/or work orders, utility data, publicly-available data, and/or user input. In some cases, data from different sources may be used to determine a value used to score of a contributing metric. For example, a filtration efficiency metric may be scored based on pressure sensor data, gas flow sensor data, a time from filter installation from building data, and/or particulate matter (PM) sensor data. Generally speaking, if the scores of multiple parameters are used to calculate a subgroup index or a composite index, the weighting factors applied to the scores total 100%. For example, the weighting factors applied to the scores of contributing metrics (also referred to herein as sub-weights) of a subgroup total 100% and/or the weighting factors applied to the scores of subgroups of a composite index total 100%. For example, a safety index may be associated with a virus risk subgroup, a security subgroup, and a safety hazard subgroup and the subgroup weighting factor applied to the virus risk subgroup may be 35%, the weighting factor applied to the security subgroup may be 35%, and the weighting factor applied to the safety hazard subgroup may be 30%, such that the subgroup weighting factors total 100%.

Table IV is an example of criteria that may be used to calculate one or more composite indices, such as a health index, a safety index, or a performance index for an enclosure. Eqns. 5a-5c and 6a-6c or Eqns. 3 and 4 may be employed to calculate a first composite index CI₁, a second composite index CI₂, and a third composite index CI₃; these indices may be a health index, the safety index, or the performance index, respectively, in some embodiments. In Table IV, the subgroups associated with the first composite index CI₁ include a GI_{1,1} subgroup, a GI_{1,2} subgroup, and a GI_{1,3} subgroup. The subgroups associated with the second composite index CI₂ include a GI_{2,1} subgroup, a GI_{2,2} subgroup, a GI_{2,3} subgroup, an GI_{2,4} subgroup, and a GI_{2,5} subgroup. The subgroups associated with the third composite index CI₃ include a GI_{3,1} subgroup, a GI_{3,2} subgroup, and an GI_{3,3} subgroup. Some examples of subgroups used to calculate a composite index include one or more of a virus risk subgroup, a security subgroup, a safety hazard subgroup, a ventilation subgroup, a filtration subgroup, a thermal comfort subgroup, an environmental satisfaction subgroup, a complaints subgroup, a utilities subgroup, a system operation subgroup, and an occupancy subgroup.

Some examples of contributing metrics in a virus risk subgroup may include an air exchange (AER) rate, a current carbon dioxide (CO₂) level, a filtration efficiency, a relative humidity (RH) level, and/or occupancy data (e.g., occupancy capacity limit). An example of a contributing metric in a security subgroup may include a ratio of building access used/issued. Some examples of contributing metrics in a safety hazard subgroup may include a carbon monoxide (CO) level and/or a volatile organic compound (VOC) level. Some examples of contributing metrics in a ventilation subgroup may include an air exchange (AER) rate, a current carbon dioxide (CO₂) level, an ozone level, a formaldehyde level, and/or a total volatile organic compound (TVOC) level. Some examples of contributing metrics in a filtration subgroup may include filtration efficiency, sensing of particulate matter of about 2.5 μm (PM_{2.5}), and/or sensing particulate matter of about 10 μm (PM₁₀). Some examples of metrics in a thermal comfort subgroup may include an ASHRAE thermal comfort, a temperature, and/or a relative humidity level. Some examples of contributing metrics in an environmental satisfaction subgroup may include a percentage (%) of occupant survey response rate, and/or a percentage (%) of occupant survey response rate. Some examples of contributing metrics in a complaints subgroup may include a number of environmental complaints from occupants. Some examples of contributing metrics in a utilities subgroup may include energy use intensity, water, steam, and/or waste. Some examples of contributing metrics in a system operation subgroup may include window (e.g., tintable window) operation and/or operating sensor uptime. Some examples of contributing metrics in an occupancy subgroup may include dwell time, percent room/floor capacity, and/or percent building capacity. In other implementations, different criteria may be used to calculate one or more composite indices. For example, in other implementations fewer, additional, and/or different subgroups may factor into the calculation of a composite index. Additionally or alternatively, in other implementations fewer, additional, or different contributing metrics may be in the subgroups.

TABLE IV
Example criteria for calculating health, safety and performance indices

| | | Weighting | | Weighting |
|---|---|---|---|---|
| | | Factor for | | Factor for |
| Composite | Subgroup | Subgroup | Contributing | Metrics |
| Index (CI) | Index (GI) | Index (GW_{i,j}) | Metrics | (W_{i,j,k}) |
| First | GI_{1,1} | GW_{1,1} | P_{1,1,1} | W_{1,1,1} |
| Composite | | | P_{1,1,2} | W_{1,1,2} |
| Index, CI₁ | | | P_{1,1,3} | W_{1,1,3} |
| | | | P_{1,1,4} | W_{1,1,4} |
| | | | P_{1,1,5} | W_{1,1,5} |
| | GI_{1,2} | GW_{1,2} | P_{1,2,1} | W_{1,2,1} |
| | GI_{1,3} | GW_{1,3} | P_{1,3,1} | W_{1,3,1} |
| | | | P_{1,3,2} | W_{1,3,2} |
| Second | GI_{2,1} | GW_{2,1} | P_{2,1,1} | W_{2,1,1} |
| Composite | | | P_{2,1,2} | W_{2,1,2} |
| Index, CI₂ | | | P_{2,1,3} | W_{2,1,3} |
| | | | P_{2,1,4} | W_{2,1,4} |
| | | | P_{2,1,5} | W_{2,1,5} |
| | GI_{2,2} | GW_{2,2} | P_{2,2,1} | W_{2,2,1} |
| | | | P_{2,2,2} | W_{2,2,2} |
| | | | P_{2,2,3} | W_{2,2,3} |
| | GI_{2,3} | GW_{2,3} | P_{2,3,1} | W_{2,3,1} |
| | | | P_{2,3,2} | W_{2,3,2} |
| | | | P_{2,3,3} | W_{2,3,3} |
| | GI_{2,4} | GW_{2,4} | P_{2,4,1} | W_{2,4,1} |
| | | | P_{2,4,2} | W_{2,4,2} |
| | GI_{2,5} | GW_{2,5} | P_{2,5,1} | W_{2,5,1} |
| | GI_{3,1} | GW_{3,1} | P_{3,1,1} | W_{3,1,1} |
| Third | | | P_{3,1,2} | W_{3,1,2} |
| Composite | | | P_{3,1,3} | W_{3,1,3} |
| Index, CI₃ | | | P_{3,1,4} | W_{3,1,4} |
| | GI_{3,2} | GW_{3,2} | P_{3,2,1} | W_{3,2,1} |
| | | | P_{3,2,2} | W_{3,2,2} |
| | GI_{3,3} | GW_{3,3} | P_{3,3,1} | W_{3,3,1} |
| | | | P_{3,3,2} | W_{3,3,2} |
| | | | P_{3,3,3} | W_{3,3,3} |

In certain implementations, a safety index may be calculated by combining weighted scores of subgroup indices associated with the safety index. Each weighted score of a subgroup index is a combination of weighted scores of contributing metrics. Some examples of contributing metrics that may be used to determine a safety index include environmental sensor data, air exchange rate, a current carbon dioxide (CO₂) level, a filtration efficiency, a relative humidity (RH) level, occupancy data such as occupancy capacity limit, and a ratio of building access used to access issued. Some examples of subgroups that may be contribute to a safety index include, an infectious disease transmissibility (also sometimes referred to herein as “virus risk”) subgroup, a security subgroup, a ventilation subgroup, and/or safety hazard subgroup. These safety index scores may be used by building managers, facility managers and/or tenants to take corrective actions to improve the safety of their buildings or to benchmark their buildings against one another. Some examples of contributing metrics in an infectious disease transmissibility subgroup include an air exchange (AER) rate, a current carbon dioxide (CO₂) level, a filtration efficiency, a relative humidity (RH) level, and/or occupancy data (e.g., occupancy capacity limit). An example of a contributing metric that may be included in a security subgroup includes a ratio of the number of building access credentials used during a period (e.g., in past month) over the total number of building access credentials issued. Some examples of contributing metrics that may be included in a safety hazard subgroup include a carbon monoxide (CO) level and a volatile organic compound (VOC) level.

One or more security-related metrics may contribute to a security subgroup. One example of a security-related contributing metric is a ratio of the number of building access credentials used during a period (e.g., in one week, one month, etc.) over the total number of building access credentials issued. A security system (e.g., security system(s) 1022 in FIG. 10) may include, for example, magnetic card access, turnstile, solenoid driven door lock, (e.g., surveillance) camera, (e.g., burglar) alarm, and/or metal detector that may be used to determine the number of building access credentials used during a period of time. In some cases, one or more sensors disposed in the enclosure may be configured to measure security-related parameters such as (e.g., glass) breakage and/or presence of unauthorized personnel. Sensors may cooperate with one or more (e.g., active) devices, such as a radar or lidar. The devices may operate to detect physical size of an enclosure, personnel present in an enclosure, stationary objects in an enclosure and/or moving objects in an enclosure.

An air exchange rate (AER) (also sometimes referred to herein as an atmosphere exchange rate) may be an indicator of an increased virus risk, ventilation risk, etc. For example, an AER can be calculated as follows: AER=[ln(C_{actual}/C_{design})]/t, where C_{actual} is the measured indoor carbon dioxide (CO₂) concentration and C_{design} refers to a maximum absolute indoor carbon dioxide (CO₂) concentration. The C_{design} can be calculated as follows: C_{design}=ΔCO₂+Cₒᵤₜ where Cₒᵤₜ refers to an ambient outside atmospheric component concentration and ΔCO₂ refers to a steady state differential concentration of CO₂. In one implementation, the AER may be determined by isolating a period of time during which the differential of the carbon dioxide (CO₂) concentration decay is linear. The AER may be calculated as the slope of the linear best fit line of the differential of the carbon dioxide (CO₂) concentration level over time. The carbon dioxide (CO₂) level decay may be linear, for example, at lunchtime or after 5 pm.

In some cases, one or more gas sensors may be disposed in, or at, the enclosure to provide sensor data (readings/measurements) of concentration levels of the one or more gases in, for example, the ambient atmosphere of the enclosure. Some examples of sensor data of concentration levels that may be taken by one or more gas sensors in an enclosure include a carbon dioxide (CO₂) level, a carbon monoxide (CO) level, an ozone level, a hydrogen sulfide level, a hydrogen level, a oxygen level, a formaldehyde level, and/or a relative humidity (RH) level.

In some implementations, one or more sensors disposed in, or at, an enclosure are VOC sensors. A VOC sensor can be specific to a VOC compound, or to a class of compounds (e.g., having similar chemical characteristic). For example, a VOC sensor can be sensitive to aldehydes, esters, thiophenes, alcohols, aromatics (e.g., benzenes and/or toluenes), or olefins. In some example, a group of sensors (e.g., sensor array) may sense a group of VOCs having different chemical characteristics. The group may comprise identified or non-identified compounds. The VOC sensor or group on sensors can output a sensed value of a particular compound, class of compounds, or group of compounds. The sensor output may be of a total (e.g., accumulated) measurements of the class, or group of compounds sensed. The sensor output may be of a total (e.g., accumulated) measurements of multiple sensor outputs of (i) individual compounds, (ii) classes of compounds, or (iii) groups of compounds. The one or more VOC sensors may output a VOC level or a total VOC (also referred to herein as TVOC) level.

A filtration efficiency may be determined using data from a pressure sensor, a gas flow sensor, time from filter installation data, and/or particulate matter (PM) sensing data. Atmosphere quality in an enclosure may depend on the use of filter(s) in an atmosphere handling system to remove various contaminants such as particulate matter (e.g., dust, soot, viruses, bacteria, and/or fungi). In some cases, particulate matter sensors may sense particulate matter of a particular size e.g., particulate matter of about 1 μm (PM₁), particulate matter of about 2.5 μm (PM_{2.5}), particulate matter of about 5 μm (PM₅), particulate matter of about 10 μm (PM₁₀), or particulate matter of about 20 μm (PM₂₀). In some cases, particulate matter sensors may sense particulate matter in a size range, e.g., between about 1 μm (PM₁) to about 20 μm (PM₂₀), between about 1 μm (PM₁) to about 5 μm (PM₅), between about 2.5 μm (PM_{2.5}) to about 10 μm (PM₁₀), or between about 5 μm (PM₅) to about 20 μm (PM₂₀). In one implementation, filtration efficiency is determined based at least in part on a ratio of indoor PM concentrations to outdoor PM concentrations. In one implementation, the filtration efficiency is determined or estimated based at least in part on outside sensing of particulate matter (e.g., particulates of about 2.5 μm (PM_{2.5})) before filtering and an inside sensing of particulate matter (e.g., PM_{2.5}) after filtering. In other implementations, (i) a ventilation rate through the filter (e.g., total volume of contaminated atmosphere treated by the filter per unit time), (ii) time lapse from past installation, (iii) gas pressure before the filter, (iv) gas pressure after the filter, (v) filter morphology, (iv) optical density of the gas before the filter, and/or (v) optical density of the gas after the filter may be used to determine the filter efficiency. In one example, filtration efficiency may be determined based on particulate matter sensed by one or more Wellstat sensors with an Air Type of “Outside Air” or “Fresh Air.” In another example, filtration efficiency may be determined based on a weather API such as Breezometer or like API that provides a value of particulate matter from about 2.5 μm (PM_{2.5}).

Occupancy data may include, for example, data related to social distancing and/or occupancy capacity limits for the enclosure. Some examples of occupancy data may include a number of occupants in the enclosure, an occupancy capacity limit, and/or a ratio of occupants in the enclosure to occupancy capacity limit. In one implementation, an occupancy capacity limit (sometimes referred to herein as “occupancy limit) may be based on building code requirements. In some cases, the number of occupants in an enclosure may be determined based on one or more sensors in or around the enclosure such as (e.g., body temperature scanners, air quality sensors, security cameras, occupancy sensors, social distancing badges, etc.), push buttons, medical testing labs, and/or information provided by occupants (e.g., through surveys or self-reporting). The occupancy capacity limit may be determined, e.g., from a building manager or building data. In one example, the occupancy capacity limit is stored in data of a digital twin, e.g., as part of a REVIT model.

In particular embodiments, one or more sensors, e.g., of a sensor ensemble (e.g., sensor ensemble 1305 shown in FIG. 13), may provide sensor data used to determined values of contributing metrics used to determine one or more composite indices. Some example of sensor data that may be used includes a temperature reading, a sensing of particulate matter, a sensing or reading of volatile organic compounds (VOCs), a measurement of electromagnetic energy, a pressure reading, an acceleration reading, a time, a radar reading, a lidar reading, a sensing of a glass breakage, a sensing or measurement of movement, and/or a gas level. In some cases, diverse sensors may be used to determine a value of a single contributing metric. For example, pressure sensor data, gas flow sensor data, and particulate matter (PM) sensor data may be used to determine a value for a filtration efficiency metric. The values of the contributing metrics may be scored and weighted, and the weighted scores used to calculate the scores of one or more of subgroup indices. The weighted scores of the subgroup indices of a composite index may then be used to calculate a composite index score.

In certain implementations, a health index may be calculated by combining weighted scores of subgroup indices associated with the health index. Each weighted score of a subgroup index is a combination of weighted scores of contributing metrics. Some examples of contributing metrics that may be contribute a health index include environmental sensor data, air exchange rate, filtration efficiency, ASHRAE Temp/RH, a current carbon dioxide (CO₂) level, a current carbon monoxide (CO) level, total volatile organic compound (TVOC) level, a formaldehyde level, an ozone level, sending of particulate matter of about 2.5 μm (PM_{2.5}), sensing of particulate matter of about 10 μm (PM₁₀), a temperature reading, a relative humidity level, a health index include a percentage (%) of occupants survey response rate, a percentage (%) of occupants survey response rate, a number of environmental complaints from occupants, acoustics data, and lighting data. The data for contributing metrics to the health index may be provided by, e.g., one or more sensors (e.g., a sensor ensemble), one or more surveys or questionnaires, and/or one or more work orders.

Some examples of subgroups or a health index include a ventilation subgroup, a filtration subgroup, a thermal comfort subgroup, an environmental satisfaction subgroup, and/or a complaint subgroup. These health index scores may be used by building managers, facility managers and/or tenants to take corrective actions to improve the health performance of their buildings or to benchmark their buildings against one another. A ventilation subgroup score is a composite score of contributing metrics associated with airborne compounds generated inside of an enclosure. Some examples of contributing metrics that may be included in a ventilation subgroup of a health index include an air exchange (AER) rate, a current carbon dioxide (CO₂) level, an Ozone level, a formaldehyde level, and/or a total volatile organic compound (TVOC) level. Some examples of contributing metrics that may be included in a filtration subgroup of a health index include filtration efficiency, sensing of particulate matter such as, for example, including particulate matter of about 2.5 μm (PM_{2.5}) and/or particulate matter of about 10 μm (PM₁₀). The sensing of the particular matter may be based on one or more sensors outside the enclosure to sense particulate matter before filtering and/or inside the enclosure to sense particular matter after filtering. Some examples of contributing metrics that may be included in a thermal comfort subgroup of a health index include a measurement of ASHRAE thermal comfort, a temperature, a radiant temperature, occupant survey responses, air speed from HVAC equipment, clothing values of occupants (CLO) and a relative humidity level. Some examples of contributing metrics that may be included in a environmental satisfaction subgroup of a health index include a percentage (%) of occupant survey response rate, and percentage (%) of occupant survey response rate. An example of a contributing metric that may be included in a complaints subgroup of a health index include a number of environmental complaints from occupants and/or tickets filed for maintenance or repair. In other implementations, other subgroups of wellness factors and/or other contributing metrics may be used to calculate a health index.

An ASHRAE thermal comfort metric may be associated with an ASHRAE standard for thermal environmental conditions for human occupancy such as, e.g., ANSI/ASHRAE Standard 55-2010. A % Occupants Survey Response Rate metric may refer to a % of occupants that completed the survey/total building occupancy. A net promotor score (NPS) may refer to the percentage (%) of respondents giving a 9 or 10 rating out of 10 on how likely they would be to recommend their workplace to a colleague. A number of environmental complaints metric may refer to a daily count of complaints made through facility management reporting system.

In certain implementations, a performance index may be calculated by combining weighted scores of subgroup indices associated with the performance index. Each weighted score of a subgroup index is a combination of weighted scores of contributing metrics. Some examples of contributing metrics that may be contribute a performance index include one or more of an energy use intensity, a water level, a steam level, a waste level, window operation data, operating sensor uptime data, dwell time data, percent room/floor capacity data, and percent building capacity data. Some examples of subgroups of a performance index include a utilities subgroup, a system operation subgroup, and/or a occupancy subgroup. These performance index scores may be used by building managers, facility managers and/or tenants to take corrective actions to improve the environmental performance of their buildings or to benchmark their buildings against one another. Some examples of contributing metrics to the utilities subgroup of the performance index include energy use intensity, water, steam, and/or waste. Some examples of contributing metrics to the system operation subgroup of the performance index include window (e.g., tintable window) operation and/or operating sensor uptime. Some examples of contributing metrics to the occupancy subgroup of the performance index include dwell time, percent room/floor capacity, and/or percent building capacity.

### Scoring Contributing Metrics and Scoring Plots

In certain implementations, a value (e.g., sensor data) of a contributing metric may be assigned a numerical score in a range (e.g., from 0 to 100 or 0 to 1). In one implementation, the high end of the range (e.g., 100) may indicate lowest risk and the low end of the range (e.g., 0) may indicate a highest risk. In another implementation, the high end of the range (e.g., 100) may indicate highest risk and the low end of the range (e.g., 0) may indicate a lowest risk. In some cases, the score of a contributing metric may be determined by employing a scoring plot (curve) for the range of metric scores over various metric values.

In certain implementations, a score (e.g. a metric score, a subgroup index score, and/or a composite index score) may be assigned a rating. For example, where the range of scores is between 0 (high risk)-100 (low risk), a score that is ≥75 may be assigned an “excellent” rating, a score of <75 and ≥50 may be assigned a “good” rating, a score of <50 and ≥25 may be assigned a “moderate” rating, and a score of <25 may be assigned a “poor” rating. In another example, where the range of scores is between 0 (low risk)-100 (high risk), a score that is ≥75 may be assigned an “poor” rating, a score of <75 and ≥50 may be assigned a “moderate” rating, a score of <50 and ≥25 may be assigned a “good” rating, and a score of <25 may be assigned a “excellent” rating. One or more ratings (e.g., rating of contributing metric, rating of subgroup, and/or rating of composite index) may be provided to the current occupant(s) of the enclosure, for example, via a digital twin (e.g., digital twin 1730). For example, an occupant may select a space of the building such as a floor or an office of the digital twin, and an indicator (e.g., a color) may be displayed on the digital twin showing the rating.

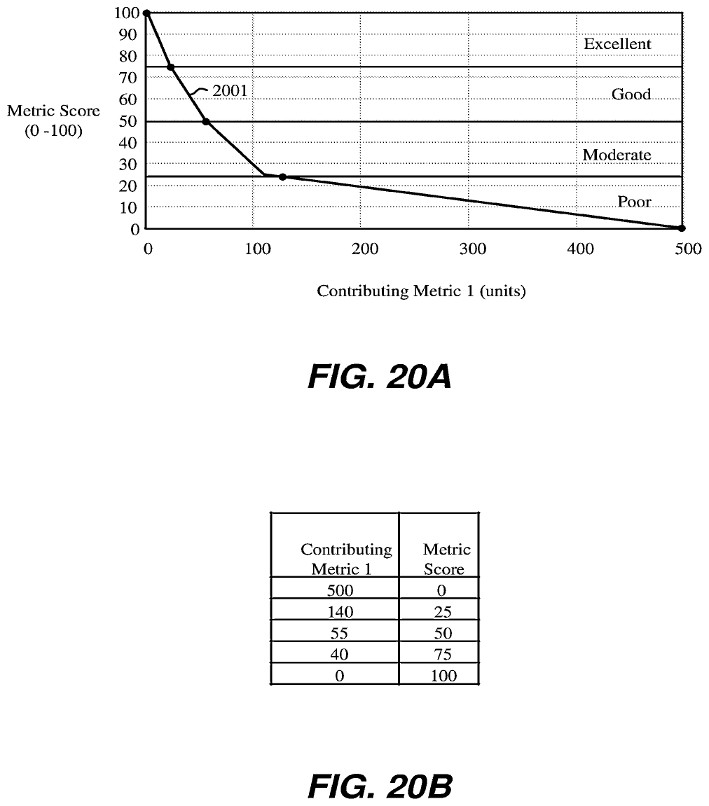

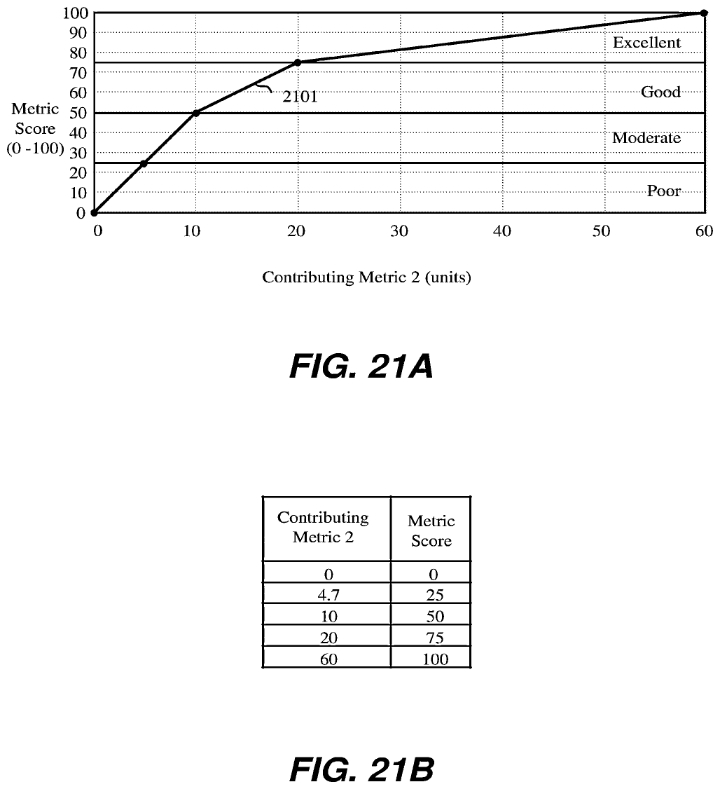

FIG. 20A depicts a graph with an example of a scoring plot 2001 that may be used to determine scores of a first contributing metric. FIG. 21A depicts a graph with an example of a scoring plot 2101 that may be used to determine a score of a second contributing metric. In these illustrated examples, the scores being assigned to the contributing metrics are in the range of 0 (high risk) to 100 (low risk). In other implementations, other ranges may be used. In FIG. 20A, the score is highest at 100 (lowest risk) when the value of contributing metric 1 is at its lowest value of 0. In FIG. 21A, the score is highest at 100 (lowest risk) when the value of contributing metric 2 is at its highest value of 60.

FIG. 20B depicts a table of an example of values 500, 140, 55, 40, and 0 of the first contributing metric associated with metric scores of 0, 25, 50, 75, and 100 derived from the scoring plot 2001 in FIG. 20A. FIG. 21B depicts a table of an example of values 0, 4.7, 10, 20, and 60 of the second contributing metric associated with metric scores of 0, 25, 50, 75, and 100 taken from the scoring plot 2101 in FIG. 21B. In FIGS. 20A and 21B, the graphs are divided into 25 point segments where a metric score of ≥75 is assigned an “excellent” rating, a metric score of <75 and ≥50 is assigned a “good” rating, a metric score of <50 and ≥25 is assigned a “moderate” rating, and a metric score of <25 is assigned a “poor” rating. In other examples, other ratings may be used. In some cases, the metric scores taken from the scoring plot may be rounded up (e.g., to the nearest 25 point segment) or the score may be truncated to a single decimal point. In some cases, the scores for the contributing metrics in a subgroup may be determined using different scoring plots. In other cases, the same scoring plot may be implemented to determine scores of multiple contributing metrics.

In some implementations, contributing metrics of a composite index are grouped into subgroups of contributing metrics. In these cases, the composite index score may be calculated using weighting factors of the subgroups (sometimes referred to herein as “weight”) and weighting factors of the contributing metrics (sometimes referred to herein as “sub-weight”). Referring to the criteria provided in Table IV, for example, to calculate a first composite index CI₁, which may be a safety index in some implementations, the scores of the subgroups GW_{1,1}, GW_{1,2} and GW_{1,3} may be calculated. The scores of each subgroup are determined by combining the weighted scores of the contributing metrics in the corresponding subgroup. In one implementation, each score of a contributing metric may be derived from a scoring plot (e.g., scoring plot 2001 in FIG. 20A and scoring plot 2101 in FIG. 21A). For instance, referring to the criteria in Table IV, the scores of the contributing metrics P_{1,1,1}, P_{1,1,2}, P_{1,1,3}, P_{1,1,4}, P_{1,1,5}, P_{1,2,1}, P_{1,3,1}, and P_{1,3,2} may be determined from eight different scoring plots using the values of the metrics. The plotted scores may be P_{1,1,1} of 50, P_{1,1,2} of 50, P_{1,1,3} of 45, P_{1,1,4} of 70, and P_{1,1,5} of 80. In one example, the weighting factors for the GI_{1,1} subgroup have equal weight of 20%. The calculated GI_{1,1} subgroup index may be calculated based on Eqn. 4 as 50×20% (P_{1,1,1})+50×20% (P_{1,1,2})+45×20% (P_{1,1,3})+70×20% (P_{1,1,4})+80×20% (P_{1,1,5})=59. In one case, a subgroup index score that is ≥75 may represent an “excellent” rating, a score of <75 and ≥50 may represent a “good” score rating, a score of <50 and ≥25 may represent a “moderate” rating, and a score of <25 may represent a “poor” rating. In this case, the calculated subgroup index score of 59 is a “good” rating.

### Overlapping Contributing Metrics

In some cases, the same metric (e.g., sensor data) may contribute to more than one subgroup index and/or more than one composite index. For example, an air exchange rate may affect both ventilation in a building and also the level of virus risk in a building. In certain implementations, composite indices may be calculated by combining weighted scores of common contributing metrics (sometimes referred to herein as overlapping metrics). For example, one or more of the same contributing metrics may be used to calculate different composite indices. The weighting factors applied to calculate the subgroup indices for the common contributing metrics may differ. For example, a carbon dioxide metric may be part of a subgroup of the health index and also part of a subgroup of the safety index. The weighting factor, W_{2,1,2}, applied to the carbon dioxide metric of the subgroup of the health index may be different from the weighting factor, W_{2,1,1}, applied to the carbon dioxide metric of the subgroup of the safety index. In some cases, each composite index is calculated using at least one common contributing metric with another composite index. In one implementation, a first composite index may be based on sensor data from a first set of one or more sensors and a second composite index may be based on sensor data from a second set of one or more sensors where the first set of sensors have at least one sensor in common with the second set of sensors.

### Dynamic Available Data

Different metric data (e.g., sensor data) may be available at different times. For example, certain sensors may not be functional at certain times of the day or on certain days, e.g., occupancy sensors may not be operational on a federal holiday. As another example, a sensor may be unavailable due to sensor malfunction or power outage. In certain implementations, techniques described herein may dynamically adjust contributing metric(s), subgroup(s), and/or weighting factor(s) used to calculate a composite index based on availability of data. In some cases, the calculated composite index may be considered agnostic of any technical data stack or individual sensor and can be adjusted based on what data is available.

In certain implementations, a system (e.g., control system 1200 in FIG. 12, ventilation system 1100 in FIG. 11, control system 1400 in FIG. 14, computing system 1500 in FIG. 15, control system 1700 in FIG. 17, system 1800 in FIG. 18, or computing system 1990 in FIG. 19) including one or more controllers and/or processor thereof may store, retrieve, or utilize certain contributing metrics depending on the availability of the data. Memory (e.g., memory 1502, electronic storage unit 1504 in FIG. 15, memory 1502 in FIG. 15, memory 1992 in FIG. 19) may store one or more metrics for the enclosure (e.g., a building or a space in the building). At least one of the stored values may be an estimated value derived from measured values for one or more (e.g., two or more) of the other metrics. These metrics may be used to calculate a composite index for the enclosure and/or determine remediation actions that will improve the scores of one or more composite indices and/or one or more subgroup indices. Provided that corresponding sensors or other data sources are available to provide the data, the one or more controllers and/or processor may obtain the data. When one or more sensors (e.g., sensor types) or other sources of the data are not available to provide the data, the one or more controllers and/or processor may dynamically adjust to use only data available, according to certain implementations.

In one implementation, for example, the one or more controllers and/or processor may omit a contributing metric that does not have data available when calculating a composite index and adjust the weighting factors of the remaining contributing metrics in the subgroup. For instance, referring to the criteria in Table IV, if data for the P_{1,1,5} metric is not available, the GI_{1,1} subgroup index may be calculated using the remaining metrics in the subgroup: P_{1,1,1}, P_{1,1,2}, P_{1,1,3} and P_{1,1,4}. The weighting factors applied to these remaining contributing metrics may be adjusted, e.g., each weighting factor increased equally. For example, if the original weighting factors were 35% for P_{1,1,1}, 35% for P_{1,1,2}, 10% for P_{1,1,3}, 10% for P_{1,1,4}, and 10% for P_{1,1,5}, the weighting factors may be increased equally (e.g., increasing each of the four remaining factors by 10%/4=2.4%) 1 to 37.5%, 37.5%, 12.5%, and 12.5% respectively. In another example, the plotted metric scores for P_{1,1,1} is 50, for P_{1,1,2} is 50, for P_{1,1,3} is 45, for P_{1,1,4} is 70, for P_{1,1,5} is 80, for P_{1,3,1} is 80, and for P_{1,3,2} is 97 and P_{1,2,1} for the GI_{1,2} subgroup is not available (e.g., not able to be calculated). In this case, the GI_{1,2} subgroup can be omitted from the calculation of the first composite index CI₁ and the weighting factors for the remaining subgroups adjusted, e.g., increased equally. For example, the score for the GI_{1,1} subgroup may be 50×20% (P_{1,1,1})+50×20% (P_{1,1,2})+45×20% (P_{1,1,3})+70×20% (P_{1,1,4})+80×20% (P_{1,1,5})=59 and for the GI_{1,3} subgroup may be 80×50% (P_{1,3,1})+97×50% (P_{1,3,2})=88.5. If the original weighting factors for these remaining subgroups were 50% (GI_{1,1}), 25% (GI_{1,2}), and 25% (GI_{1,3}), upon omission of GI_{1,2} subgroup, the weighting factors of the remaining subgroups may be increased equally to 62.5 and 37.5% and the first composite index CI₁ score may be 59×62.5%+88.5×37.5%=70.06. In one case, a subgroup index score that is >75 may represent an “excellent” rating, a score of <75 and ≥50 may represent a “good” score rating, a score of <50 and ≥25 may represent a “moderate” rating, and a score of <25 may represent a “poor” rating. In this case, the calculated subgroup index score of 70.06 is a “good” rating. In one example, a notification of the unavailable data may be sent to the occupant(s).

In another example, when data is unavailable, the one or more controllers and/or processor may use historical data, predicted or projected values, and/or third party data. In one aspect, a learning algorithm may utilize historical data as a learning set to predict values in the enclosure.

Alternatively, when data is unavailable, the one or more controllers and/or processor may not calculate the composite index and/or return a notification to the user indicating that the composite index is not calculated and/or indicating which data or sensors are not available. For example, if the unavailable data is part of a minimum data set needed to calculate the composite index or a subgroup score, the one or more controllers and/or processor may not calculate the score and/or may return a notification to the user. An example of a minimum data set for calculating a virus risk subgroup index may be an air exchange rate, a CO₂ concentration level, and a filtration efficiency; in another example, the minimum data set may be a CO₂ concentration level, a filtration efficiency, and relative humidity level; in yet another example, the minimum data set may include an air exchange rate, a CO₂ concentration level, a filtration efficiency and relative humidity level. An example of a minimum data set for calculating a ventilation subgroup index may be an air exchange rate, and a TVOC level; in another example the minimum data set may be a CO₂ concentration level; in yet another example, the minimum data set may be an air exchange rate, a CO₂ concentration level, and a TVOC level. An example of a minimum data set for calculating a filtration subgroup index may be a filtration efficiency and a sensing of particulate matter of about 2.5 μm (PM_{2.5}); in another example, the minimum data set may include a filtration subgroup index, a sensing of particulate matter of about 2.5 μm (PM_{2.5}), and a sensing of particulate matter of about 10 μm (PM₁₀); in yet another example, the minimum data set may include a sensing of particulate matter of about 2.5 μm (PM_{2.5}) and a sensing of particulate matter of about 10 μm (PM₁₀). An example of a minimum data set for calculating a thermal comfort subgroup index may be an ASHRAE thermal comfort level and a relative humidity; in another example the minimum data set may include a temperature and a relative humidity level; in yet another example, the minimum data set may include an ASHRAE thermal comfort level, a temperature, and a relative humidity level. An example of a minimum data set for calculating a utilities subgroup index may be energy use intensity. An example of a minimum data set for calculating a system operation subgroup index may be operating sensor uptime or operating sensor downtime. An example of a minimum data set for calculating an occupancy subgroup index may be percent building capacity or capacity of a particular space in the building, such as a conference room.

In certain implementations, trends over time of one or more of a composite index score, a contributing metric score, a contributing metric value, or a subgroup score and/or trends of other data may be determined. For example, a system (e.g., control system 1200 in FIG. 12, ventilation system 1100 in FIG. 11, control system 1400 in FIG. 14, computing system 1500 in FIG. 15, control system 1700 in FIG. 17, system 1800 in FIG. 18, or computing system 1990 in FIG. 19) including one or more controllers and/or processor thereof may store composite index scores, contributing metrics scores and values, and/or subgroup index scores over time (e.g., based on time of sensor reading or time data taken). The stored data may be retrieved and used to calculate trends such as a rate of change over time and/or a overall trend (e.g., a downward trend, an upward trend, a sharp increase, a sharp decline, cyclic fluctuating, random fluctuating, and no change) in the data. In some cases, the calculated trends may be assigned a rating such as, for example, “improving,” “worsening,” or “staying the same.”

### User Interface (e.g., Digital Twin) and Remediation Actions

A user (e.g., an occupant of a building) may navigate within a digital twin (e.g., digital twin 1601 in FIG. 16 or virtual building model 1730 in FIG. 17) or other user interface to view or be notified of, for example, real-time scores of composite indices, real-time scores of contributing metrics, real-time scores of subgroup indices, current values of contributing metrics, current ratings (e.g., “Excellent,” “Good,” “Moderate,” and “Poor”) of composite indices, current ratings of contributing metrics, current ratings of contributing metrics, current values of contributing metrics, and trends in scores, ratings, and values. For example, a user may navigate through a digital twin of a facility such as a building to select a virtual portion of the facility such as a virtual floor (e.g., floor 1602 of digital twin 1601 as depicted in FIG. 16) or a tenant area and/or select a virtual space (e.g., virtual space 1738 in FIG. 17) in the facility to view the virtual portion or space of the facility and/or to view the real-time data associated with that portion or space. For example, a user may navigate through a digital twin of a building to select a floor to receive real-time data associated with the floor and then select different offices or other spaces on the floor to receive real-time data associated with those spaces. In some cases, the data may be provided through color coding of different ranges of values of the data. For example, ratings of scores may be color coded (e.g., red assigned to “Poor” rating, yellow assigned to a “Moderate” rating, blue assigned to a “Good” rating, and green assigned to an “Excellent” rating). For example, a user may select a space in, or portion of, the virtual facility and the space or portion may be displayed in a color based on the current rating and accompanied by a label with the type of score, for example, a floor may be displayed in red with notification that “This floor currently has a Poor Rating for a Health Index”). As another example, upon selecting the space or portion of the virtual facility, a visual display of data such as a dashboard may be provided to the user with the real-time data. In some cases, the source of the problem for a low rating may be displayed and/or a notification one or more remediation actions to improve scores may be provided to the user. In one example, upon navigating to a virtual space of a facility, a user may be provided with warnings of any data with current low ratings.

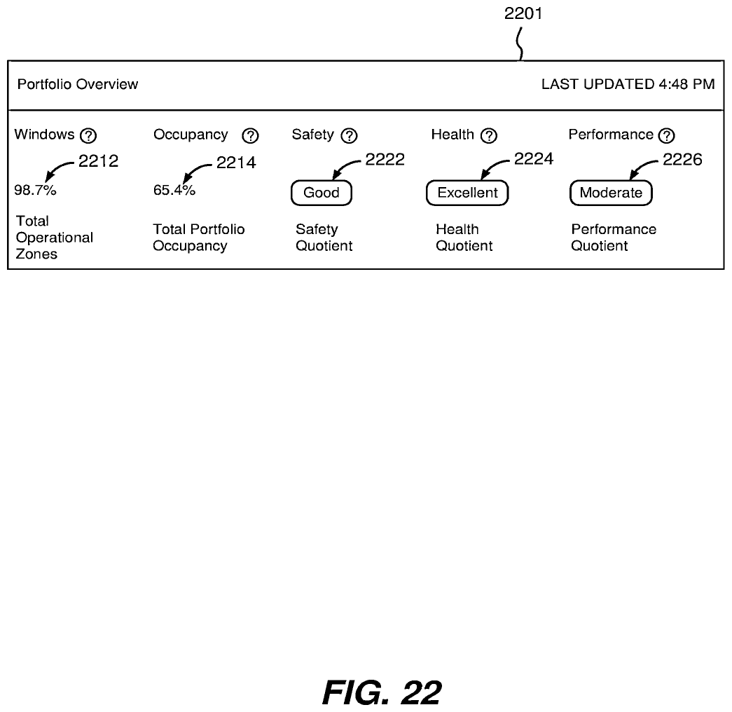

FIG. 22 is an example of a dashboard display 2201 with real-time data for a facility such as a building, in accordance with some embodiments. In one example, the dashboard display 2201 may be provided to a user on a user interface device when the user selects a digital twin (e.g., digital twin 1601 in FIG. 16 or virtual building model 1730 in FIG. 17) of the facility. The dashboard display includes a current number of operational zones (e.g., areas) of the facility 2212, the current subgroup occupancy index score 2214, a current safety index score 2222, a current health index score 2224, and a current performance index score 2226.

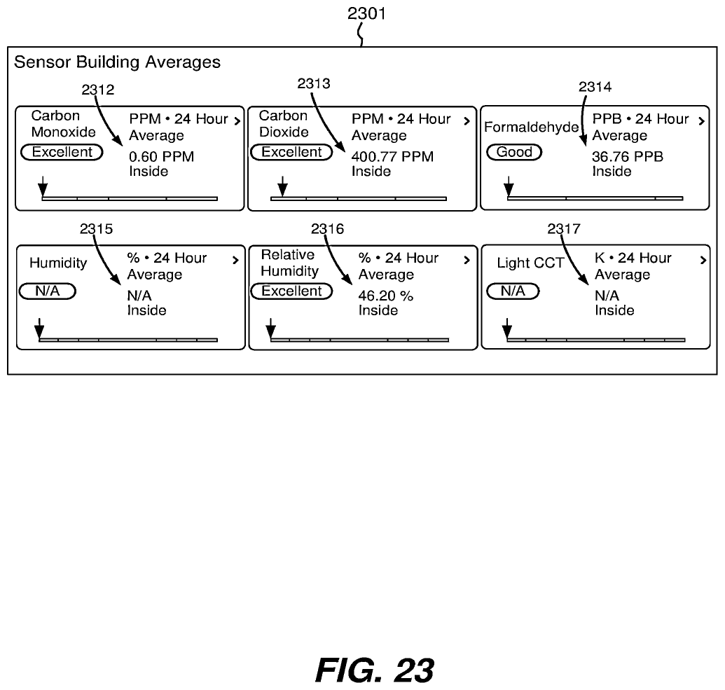

FIG. 23 is an example of a dashboard display 2301 with real-time data for a space of a facility, in accordance with some embodiments. In one example, the dashboard display 2301 may be provided to a user on a user interface device when the user selects a virtual space in the facility on a digital twin (e.g., digital twin 1601 in FIG. 16 or virtual building model 1730 in FIG. 17) of the facility. For example, the user may select a virtual floor (e.g., floor 1602 of digital twin 1601 as depicted in FIG. 16), a tenant area, or a virtual space (e.g., virtual space 1738 in FIG. 17) of the digital twin. The dashboard display 2301 includes a current value of a carbon monoxide (CO) level 2312, a current value of a carbon dioxide (CO₂) level 2313, a current value of a formaldehyde level 2314, a current value of a humidity level 2315, a current value of a relative humidity level 2316, and a light correlated color temperature in Kelvin (CCT) level 2317. One or more of these values may be provided by one or more sensors, e.g., a sensor ensemble of two or more environmental sensors.

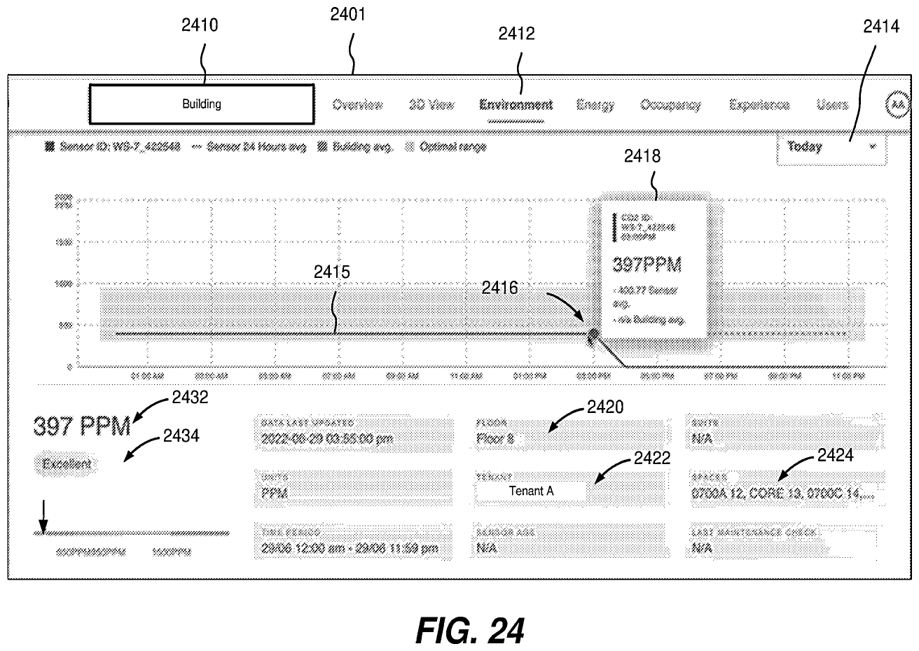

FIG. 24 is an example of a dashboard display 2401 with real-time data for a carbon dioxide (CO₂) level metric for spaces of a tenant in a facility, in accordance with some embodiments. In one example, dashboard display 2401 may be provided to a user (e.g., a tenant or other occupant) on a user interface device when the user selects a tenant name in a digital twin (e.g., digital twin 1601 in FIG. 16 or virtual building model 1730 in FIG. 17) of the facility and requests real-time data for the carbon dioxide (CO₂) level metric via the digital twin. The dashboard display 2301 includes the facility 2410, the environmental condition 2412, the current date 2414, the trend of the carbon dioxide (CO₂) levels over time 2415 showing the current level 2416, carbon dioxide (CO₂) sensor information 2418, a current value of the carbon dioxide (CO₂) level 2432, a rating of the carbon dioxide (CO₂) level 2434, a floor of the facility 2420, a tenant name 2422, and tenant spaces (e.g., enclosures) 2424.

Occupants, building managers, or building maintenance personnel may take one or more remediation actions around managing staff, resources, and one or more building systems to improve, e.g., composite index (ices), contributing metric(s), and/or subgroup index (ices) scores. Some examples of remediation actions include: (i) sending notification to occupants to vacate a space or a building or move to specific portions of the building to reduce occupancy and/or to be able to perform maintenance of cleaning, (ii) sending notification to occupants requiring use of protective equipment, (iii) performing maintenance or cleaning on one or more contaminated or damaged building components or areas, (iv) adjusting HVAC flow, (v) adjusting temperature of an enclosure, (vi) replacing one or more air filters, and (vii) adjusting lighting. For example, when a filtration efficiency metric value falls below a predetermined threshold from its nominal efficiency, e.g., has a “poor” rating, an air filter may be replaced as a remediation action. As another example, when a virus risk index of an enclosure score falls below a predetermined threshold associated with an acceptable risk, a notification may be sent to the occupants to vacate the building and/or to work from home as a remediation action and a notification sent to the building manager to perform cleaning of the enclosure. As another example, when a virus risk index score is exhibiting a trend to higher risk, a building manager may adjust the HVAC to increase flow to increase the air exchange rate. As another example, when a carbon monoxide level or VOC level is exhibiting a trend toward a higher risk but is still at an acceptable rating such as “good” rating, a building manager may adjust the HVAC to increase flow to increase the air exchange rate. As another example, when thermal comfort subgroup index score of an enclosure is rated as “poor,” a building manager may be notified to adjust controls on the HVAC system and/or transition tint of one or more tintable windows (e.g., electrochromic windows) to improve the thermal comfort rating. As another example, when the current temperature of an enclosure increases above a threshold, the building manager may be notified to transition tint of one or more tintable windows (e.g., electrochromic windows) in the enclosure to a darker tint state.

In certain implementations, a user (e.g., occupant) may interact with a building manger via the digital twin, for example, to notify the building manager of a problem, to receive notifications regarding the composite index, subgroup, and metric scores or other data and/or to take remediation measures to improve the scores. A notification to a building manager or an occupant may be generated as a warning message delivered immediately or may be included in a periodically generated report, for example. For example, when filtration efficiency declines below a predetermined threshold from its nominal efficiency, a building manager may be sent an immediate notification to perform a corrective action such as replacing an air filter.

In some cases, a display to a user may include current status and sensor data from one or more sensors (e.g., carbon dioxide (CO₂) sensor information 2418 in FIG. 24) and/or current operational data of one or more building systems (e.g., security system(s) 1022, an HVAC system 1023, a lighting system 1024, a power system(s) 1026, an elevator(s) 1027, a fire system(s) 1028 in FIG. 10) of a facility. The user may generate a ticket or other notification, e.g., via a digital twin, to a building manager of the facility that a sensor is not functioning properly or that one or more building systems is not functioning properly.

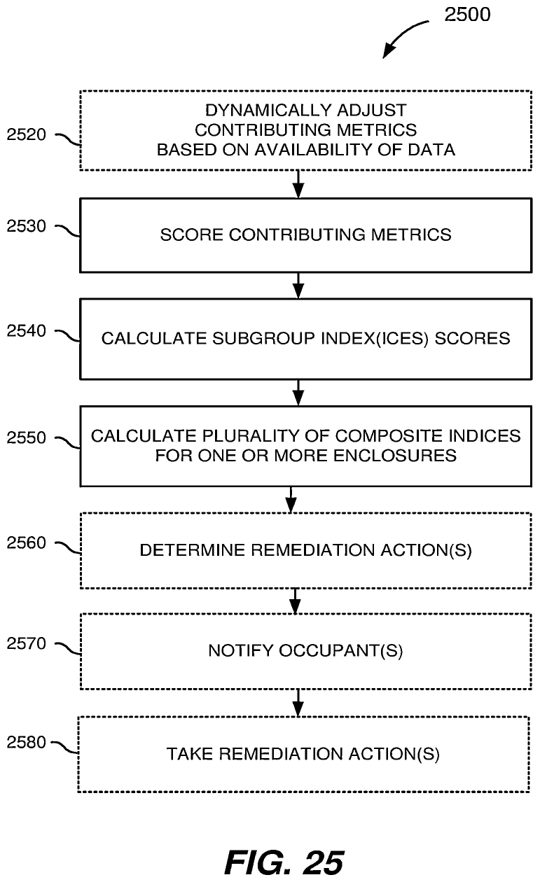

FIG. 25 is a flowchart 2500 depicting an example of a method of determining a plurality of composite indices for one or more enclosure and/or controlling one or more building systems based on a plurality of composite indices, in accordance with various embodiments. The operations shown in flowchart 2500 are for illustration purposes only and are not intended to be limiting. In various implementations, modifications may be made to flowchart 2500 to add or omit one or more operations or change the order of operations. The operations in flowchart 2500 may be performed using a system such as, e.g., control system 1200 in FIG. 12, ventilation system 1100 in FIG. 11, control system 1400 in FIG. 14, computing system 1500 in FIG. 15, control system 1700 in FIG. 17, system 1800 in FIG. 18, or computing system 1990 in FIG. 19.

At optional (denoted by dashed line) operation 2520, the system may dynamically adjust contributing metric(s), subgroup(s), and/or weighting factor(s) used to calculate a composite index based on availability of data. For example, different data for contributing metrics (e.g., sensor data) may be available at different times. For instance, sensors may become unavailable due to malfunction or power outage. The availability of the data may be determined, for example, based on a current value (e.g., sensor reading) of the contributing metric. For example, sensor data for a sensor may be unavailable if a current sensor reading is below a threshold functioning level, there is no sensor reading, or an error message is received. Provided that corresponding sensors or other data sources are available to provide the data, the one or more controllers and/or processor may obtain the data from memory utilize the data to determine the plurality of composite indices. When the one or more sensors or other sources of the data are not available to provide the data, the one or more controllers and/or processor may dynamically adjust to use only data available or may provide a notification to the user. In one implementation, for example, the one or more controllers and/or processor may omit a contributing metric that does not have data available when calculating a composite index and adjust the weighting factors of the remaining contributing metrics in the subgroup. For example, referring to the criteria in Table IV, if data for the occupancy capacity limit metric is not available, the virus risk subgroup index may be calculated using the remaining metrics in the subgroup and the weighting factors applied to these remaining contributing metrics adjusted, e.g., each weighting factor increased equally. A notification of the unavailability of data may be sent to the occupant(s). In another implementation, the one or more controllers and/or processor may use historical data, predicted or projected values, and/or third party data. In one aspect, a learning algorithm may utilize historical data as a learning set to predict values in the enclosure. In yet another implementation, the one or more controllers and/or processor may not calculate the composite index and/or return a notification to the user indicating that the composite index is not calculated and/or indicating which data or sensors are not available.

At operation 2530, the system may score values of the one or more contributing metrics that may be used to calculate the plurality of composite indices. A contributing metric may be assigned a numerical score in a range (e.g., from 0 to 100 or 0 to 1). In one implementation, the high end of the range (e.g., 100) may indicate lowest risk and the low end of the range (e.g., 0) may indicate a highest risk. In another implementation, the high end of the range (e.g., 100) may indicate highest risk and the low end of the range (e.g., 0) may indicate a lowest risk. In one implementation, a score of each contributing metric may be determined by employing a scoring plot (curve) (e.g., scoring plot 2001 in FIG. 20A or scoring plot 2101 in FIG. 21A) for a range of metric scores over various metric values. For example, using the scoring plot 2001 in FIG. 20A, the corresponding metric scores for the values 500, 140, 55, 40, and 0 of a first contributing metric are: 0, 25, 50, 75, and 100 derived from the scoring plot 2001 in FIG. 20A.

In some implementations, contributing metrics of a composite index are grouped into subgroups of contributing metrics. The composite index score may be calculated using weighting factors of the subgroups and weighting factors of the contributing metrics. Each subgroup index score may be calculated by combining weighted scores of contributing metrics associated with the subgroup using Eqn. 4.

At operation 2540, the system may calculate each subgroup index score by combining weighted scores of contributing metrics associated with the subgroup using Eqn. 4. For example, referring to the criteria in Table IV, the scores of the contributing metrics to the GI_{1,1} subgroup includes P_{1,1,1}, P_{1,1,2}, P_{1,1,3}, P_{1,1,4}, and P_{1,1,5}. If, for example, the score of the current P_{1,1,1} is 50, the score of P_{1,1,2} is 50, the score of the P_{1,1,3} is 45, the score of P_{1,1,4} is 70, and the score of P_{1,1,5} is 80, and the weighting factors have equal weight of 20%, the calculated GI_{1,1} subgroup index may be calculated based on Eqn. 4 as 50×20% (P_{1,1,1})+50×20% (P_{1,1,2})+45×20% (P_{1,1,3})+70×20% (P_{1,1,4})+80×20% (P_{1,1,5})=59.

At operation 2550, the system may calculate a plurality of composite indices for one or more enclosures. For example, a safety index, a health index, and a performance index may be calculated. Each composite index score may be calculated using weighting factors and scores of the subgroup index (ices) associated with the composite index using Eqn. 3. The safety index may be calculated by combining the weighted scores of the subgroup indices associated with the safety index. The safety index may be calculated using weighting factors and scores of the subgroups associated with the health index using Eqn. 5a. The health index may be calculated by combining the weighted scores of the subgroup indices associated with the health index. The health index may be calculated using weighting factors and scores of the subgroups associated with the health index using Eqn. 5b. The performance index may be calculated by combining the weighted scores of the subgroup indices associated with the performance index. The health index may be calculated using weighting factors and scores of the subgroups associated with the performance index using Eqn. 5c.

At operation 2560, the system may determine one or more remediation actions around managing staff, resources, and one or more building systems to improve a metric score, a subgroup index score, and/or a composite index score. Some examples of remediation actions include: (i) sending notification to occupants to vacate a space or a building or move to specific portions of the building to reduce occupancy and/or to be able to perform maintenance of cleaning, (ii) sending notification to occupants requiring use of protective equipment, (iii) performing maintenance or cleaning on one or more contaminated or damaged building components or areas, (iv) adjusting HVAC flow, (v) adjusting temperature of an enclosure, (vi) replacing one or more air filters, and (vii) adjusting lighting. To determine a remediation action, the system may, for example, determine that a metric score, a subgroup index score, and/or a composite index score is below an acceptable threshold level and then determine an action around managing staff, resources, and one or more building systems that will improve the score. For example, when a filtration efficiency metric value falls below a predetermined threshold from its nominal efficiency, e.g., has a “poor” rating, an air filter may be replaced as a remediation action. As another example, when a virus risk index of an enclosure score falls below a predetermined threshold associated with an acceptable risk, a notification may be sent to the occupants to vacate the building and/or to work from home as a remediation action and a notification sent to the building manager to perform cleaning of the enclosure. As another example, when a virus risk index score is exhibiting a trend to higher risk, a building manager may adjust the HVAC to increase flow to increase the air exchange rate. As another example, when a carbon monoxide level or VOC level is exhibiting a trend toward a higher risk but is still at an acceptable rating such as “good” rating, a building manager may adjust the HVAC to increase flow to increase the air exchange rate. As another example, when thermal comfort subgroup index score of an enclosure is rated as “poor,” a building manager may be notified to adjust controls on the HVAC system and/or transition tint of one or more tintable windows (e.g., electrochromic windows) to improve the thermal comfort rating. As another example, when the current temperature of an enclosure increases above a threshold, the building manager may be notified to transition tint of one or more tintable windows (e.g., electrochromic windows) in the enclosure to a darker tint state. In some cases, a lookup table of actions that improve scores may be used.

At operation 2570, occupant(s) may be notified of a metric score, value, or rating, a subgroup index score or rating, and/or a composite index score or rating. In addition or alternatively, the occupant(s) may be notified of remediation action(s) that may improved scores and/or receive information regarding trends in metric, subgroup, or composite index scores. In one example, an occupant may navigate within a digital twin (e.g., digital twin 1601 in FIG. 16 or virtual building model 1730 in FIG. 17) or other user interface to select a virtual space and receive notifications of real-time scores of composite indices, contributing metrics, or subgroup indices, current values of contributing metrics, current ratings of composite indices, contributing metrics, or contributing metrics, current values of contributing metrics, and trends in scores, ratings, and values. In some cases, upon selecting a virtual space, visual display (e.g., dashboard 2201 in FIG. 22, dashboard 2301 in FIG. 23, and dashboard 2401 in FIG. 24) of data may be provided to the user with real-time data. In some cases, the source of the problem for a low rating may be displayed and/or a notification one or more remediation actions to improve scores may be provided to the user. In one example, upon navigating to a virtual space of a facility, a user may be provided with warnings of any data with current low ratings.

At operation 2580, occupant(s), building manager(s), and/or building maintenance personnel may take one or more remediation actions around managing staff, resources, and one or more building systems to improve, e.g., composite index (ices), contributing metric(s), and/or subgroup index (ices) scores. For example, occupants may vacate a space or a building or move to specific portions of the building to reduce occupancy and/or to be able to perform maintenance of cleaning. As another example, occupants may be required to use protective equipment such as masks. As another example, a work order may be sent to perform maintenance or cleaning on one or more contaminated or damaged building components or areas. As another example, the HVAC system may be adjusted to increase air flow. As another example, an air filter may be replaced if filtration efficiency is below a certain level. As another example, the tint state of one or more tintable windows in a building may be adjusted to change the heat load into a building and the light level in the building. For instance, the system may send control instructions to increase tint level of the one or more tintable windows to reduce heat load and the light level. Alternatively, system may send control instructions to decrease tint level of, or clear, the one or more tintable windows to increase heat load into the building and increase the light level.

Certain embodiments include a system having a plurality of sensors at a building and configured to generate sensor data associated with an environment in the building, at least one controller, and a user interface. The at least one controller is configured to receive the sensor data, and to determine, based at least in part on the sensor data, a first composite index and a second composite index of a space of the building. The user interface is configured to present information associated with the first composite index and the second composite index. In some cases, (a) the user interface may be a part of a mobile device, a laptop, a computer display, and a wall interface, (b) the first composite index may be one of a safety index, a health index, and a performance index, and the second composite index is a different index of the safety index, the performance index, and the health index, (c) the plurality of sensors includes two or more of a carbon dioxide sensor, a carbon monoxide sensor, a relative humidity sensor, a volatile organic compound (VOC) sensor, an ozone sensor, a formaldehyde sensor, a particulate sensor configured to detect PM2.5, a particulate sensor configured to detect PM10, a noise sensor, a visible light sensor, a temperature sensor, a motion sensor, an infrared sensor, a radon sensor, a hydrogen sulfide sensor, a radio frequency sensor, a microwave sensor, an ultraviolet radiation sensor, and a sensor for detecting breakage or unauthorized presence of personnel in an area, and/or (d) the determination of the first and second composite indices is further based on an air exchange rate, a filtration efficiency, an occupancy capacity limit, a ratio of building access used/issued, % occupant survey response rate, net promotor score, a number of environmental complaints, an energy use intensity, a water level, a steam level, and a waste level, a window operation level, an operating sensor uptime level, a dwell time, a percent room/floor capacity, a percent building capacity, and an ASHRAE thermal comfort.

Certain embodiments include a system having at least one controller configured to receive sensor data taken by a plurality of sensors and determine, based at least on part on the sensor data, a plurality of composite indices of one or more enclosures. In one embodiment, the at least one controller is further configured to control one or more building systems based on the plurality of composite indices. In one embodiment, the plurality of composite indices includes a safety index, a health index, and a performance index. In one embodiment, the one or more enclosures includes a building in which, or at which, the plurality of sensors is disposed and/or a space in the building. In one embodiment, the plurality of sensors includes a sensor ensemble having two or more environmental sensors. In one embodiment, the plurality of composite indices is determined based also at least in part on data from one or more of a survey, a questionnaire, a work order, or user input. In one embodiment, at least one of the composite indices is determined based on weighted scores of values of contributing metrics including the sensor data from the plurality of sensors. In one embodiment, the plurality of composite indices is determined based on weighted scores of a plurality of subgroup indices, and each weighted score of the corresponding subgroup index is determined based on weighted scores of values of contributing metrics including the sensor data from the plurality of sensors. In some cases, the at least one controller is further configured to determine a first composite index of the plurality of composite indices based on weighted scores of values of a first set of contributing metrics and determine a second composite index of the plurality of composite indices based on weighted scores of values of a second set of contributing metrics, and the first set of contributing metrics and the second set of contributing metrics include the sensor data from the plurality of sensors. In one case, the first set of contributing metrics overlaps with the second set of contributing metrics. In one case, the first set of contributing metrics only partially overlaps with the second set of contributing metrics. In one case, one or both of the first set of contributing metrics and the second set of contributing metrics is dynamically adjusted based on availability of sensor data from the plurality of sensors. In one case, the at least one controller is further configured to determine a third composite index, and the third composite index is based on weighted scores of values of a third set of contributing metrics. For instance, the third set of contributing metrics may not overlap with the first set of contributing metrics or the second set of contributing metrics. In one case, the at least one controller is configured to dynamically calculate the plurality of composite indices based on currently available data. In one case, the at least one controller is further configured to (a) determine whether data for a set of contributing metrics is available, (b) if data for a contributing metric of the set of contributing metrics is not available, remove the contributing metric from the set of contributing metrics and adjust each weighting factor for each remaining contributing metric in the set of contributing metrics, and (c) determine at least one of the composite indices based on the remaining contributing metrics and corresponding adjusted weighting factors. In one embodiment, the at least one controller is further configured to determine availability of data for a minimum set of contributing metrics. For instance, the minimum set of contributing metrics may include (A) an air exchange rate, a carbon dioxide (CO2) level, a filtration efficiency, and a relative humidity level, or (B) an air exchange rate, a carbon dioxide (CO2) level, a total volatile organic compound level, a filtration efficiency, a sensing of particulate matter of about 2.5 μm, a sending of particulate matter of about 10 μm, a thermal comfort level, a temperature reading, and a relative humidity level, or (C) an energy use intensity level, an operating sensor uptime level, and a percentage building capacity level. Additionally or alternatively, the at least one controller may be further configured to send a notification to at least one occupant of the one or more enclosures in which, or at which, the plurality of sensors is disposed if any data for the minimum set of contributing metrics is not available. In one embodiment, the plurality of composite indices is determined based on weighted scores of values of contributing metrics comprising an air exchange rate, a carbon dioxide (CO₂) level, a filtration efficiency, a relative humidity level, an occupancy capacity limit, and a ratio of building access used/issued, a carbon monoxide level, and a volatile organic compound level. In one embodiment, the plurality of composite indices is determined based on weighted scores of values of contributing metrics comprising an air exchange rate, a carbon dioxide (CO₂) level, an ozone level, a formaldehyde level, a total volatile organic compound level, a filtration efficiency, a sensing of particulate matter of about 2.5 μm, a sensing of particulate matter of about 10 μm, a thermal comfort level, a temperature reading, a relative humidity level, a percentage occupant survey response rate, a net promotor score, and a number of environmental complaints. In one embodiment, the plurality of composite indices is determined based on weighted scores of values of contributing metrics comprising an energy use intensity, a water usage rate, a steam usage rate, a waste disposal rate, a window operation level, an operating sensor uptime rate, a dwell time, a percentage room/floor capacity, and a percentage of building capacity. In one embodiment, the at least one controller is further configured to determine a first composite index, a second composite index, and a third composite index of the plurality of composite indices. In this embodiment, (A) the first composite index may be determined based on weighted scores of values of contributing metrics comprising an air exchange rate, a carbon dioxide (CO2) level, a filtration efficiency, a relative humidity level, an occupancy capacity limit, and a ratio of building access used/issued, a carbon monoxide level, and a volatile organic compound level, (B) the second composite index may determined based on weighted scores of values of contributing metrics comprising an air exchange rate, a carbon dioxide (CO2) level, an ozone level, a formaldehyde level, a total volatile organic compound level, a filtration efficiency, a sensing of particulate matter of about 2.5 μm, a sensing of particulate matter of about 10 μm, a thermal comfort level, a temperature reading, a relative humidity level, a percentage occupant survey response rate, a net promotor score, and a number of environmental complaints, and (C) the third composite index is determined based on weighted scores of values of contributing metrics comprising an energy use intensity, a water usage rate, a steam usage rate, a waste disposal rate, a window operation level, an operating sensor uptime rate, a dwell time, a percentage room/floor capacity, and a percentage of building capacity. In one embodiment, wherein the at least one controller is further configured to send control instructions for adjusting one or more building systems to improve the plurality of composite indices. In one embodiment, the at least one controller is configured to determine one or more actions configured to improve the plurality of composite indices. In this embodiment, the at least one controller may be configured to send a notification of the one or more actions to one or more occupants of the one or more enclosures in which, or at which, the plurality of sensors is disposed, for example, the at least one controller may be configured to send the notification to a digital twin of the one or more enclosures.

Certain embodiments pertain to a method that includes obtaining sensor data taken by a plurality of sensors and determining a plurality of composite indices of one or more enclosures based at least in part on sensor data from the plurality of sensors. Certain methods pertain to a non-transitory computer program product having a computer readable memory storing computer executable instructions, when read by one or more processors operatively coupled to one or more building systems, cause the one or more processors to execute operations according to any method of these embodiments. In one embodiment, the method also includes controlling one or more building systems based on the plurality of composite indices. In one embodiment, the plurality of composite indices includes a safety index, a health index, and a performance index of the one or more enclosures. The one or more enclosures may include a building in which, or at which, the plurality of sensors is disposed and/or a space in the building. In one embodiment, the plurality of sensors includes a sensor ensemble having two or more environmental sensors. In one embodiment, the determination of the plurality of composite indices is also based at least in part on data from one or more of a survey, a questionnaire, a work order, or user input. In one embodiment, the method also includes determining the plurality of composite indices based on weighted scores of values of contributing metrics including the sensor data from the plurality of sensors. In one embodiment, the method also includes determining a weighted score for each contributing metric associated with the plurality of composite indices, determining a weighted score for each subgroup index of a plurality of subgroup indices by combining weighted scores of a set of contributing metrics associated with the subgroup index, and determining each composite index of the plurality of composite indices by combining weighted scores of subgroup indices associated with the composite index. In one embodiment, the method also includes dynamically calculate the plurality of composite indices based on currently available data. In one embodiment, the method also includes (A) determining whether data for a set of contributing metrics is available, (B) if data for a contributing metric of the set of contributing metrics is not available, removing the contributing metric from the set of contributing metrics and adjusting each weighting factor for each remaining contributing metric in the set of contributing metrics (C) determining the plurality of composite indices based on the remaining contributing metrics and corresponding adjusted weighting factors. In one embodiment, the method also includes determining whether data for a minimum set of contributing metrics is available. The minimum set of contributing metrics may include (A) an air exchange rate, a carbon dioxide (CO2) level, a filtration efficiency, and a relative humidity level; or (B) an air exchange rate, a carbon dioxide (CO2) level, a total volatile organic compound level, a filtration efficiency, a sensing of particulate matter of about 2.5 μm, a sending of particulate matter of about 10 μm, a thermal comfort level, a temperature reading, and a relative humidity level; or (C) an energy use intensity level, an operating sensor uptime level, and a percentage building capacity level. In one instance, the method further includes sending a notification to at least one occupant of an enclosure of the one or more enclosures in which, or at which, the plurality of sensors is disposed if any data for the minimum set of contributing metrics is not available. In one embodiment, at least one of the composite indices is determined based on weighted scores of values of contributing metrics comprising an air exchange rate, a carbon dioxide (CO₂) level, a filtration efficiency, a relative humidity level, an occupancy capacity limit, and a ratio of building access used/issued, a carbon monoxide level, and a volatile organic compound level. In one embodiment, at least one of the composite indices is determined based on weighted scores of values of contributing metrics comprising an air exchange rate, a carbon dioxide (CO₂) level, an ozone level, a formaldehyde level, a total volatile organic compound level, a filtration efficiency, a sensing of particulate matter of about 2.5 μm, a sensing of particulate matter of about 10 μm, a thermal comfort level, a temperature reading, a relative humidity level, a percentage occupant survey response rate, a net promotor score, and a number of environmental complaints. In one embodiment, at least one of the composite indices is determined based on weighted scores of values of contributing metrics comprising an energy use intensity, a water usage rate, a steam usage rate, a waste disposal rate, a window operation level, an operating sensor uptime rate, a dwell time, a percentage room/floor capacity, and a percentage of building capacity. In one embodiment, the method also includes (A) determining a first composite index of the plurality of composite indices based on weighted scores of values of contributing metrics comprising an air exchange rate, a carbon dioxide (CO₂) level, a filtration efficiency, a relative humidity level, an occupancy capacity limit, and a ratio of building access used/issued, a carbon monoxide level, and a volatile organic compound level (B), determining a second composite index of the plurality of composite indices based on weighted scores of values of contributing metrics comprising an air exchange rate, a carbon dioxide (CO₂) level, an ozone level, a formaldehyde level, a total volatile organic compound level, a filtration efficiency, a sensing of particulate matter of about 2.5 μm, a sensing of particulate matter of about 10 μm, a thermal comfort level, a temperature reading, a relative humidity level, a percentage occupant survey response rate, a net promotor score, and a number of environmental complaints, and (C) determining a third composite index of the plurality of composite indices based on weighted scores of values of contributing metrics comprising an energy use intensity, a water usage rate, a steam usage rate, a waste disposal rate, a window operation level, an operating sensor uptime rate, a dwell time, a percentage room/floor capacity, and a percentage of building capacity. In this embodiment, the method may also sending control instructions to adjust the one or more building systems to improve the plurality of composite indices. In one embodiment, the method further includes sending control instructions to transition tint of one or more tintable windows to improve the plurality of composite indices. In one embodiment, the method further includes determining one or more actions configured to improve the plurality of composite indices. In one instance, the method further includes sending the notification to a digital twin of the one or more enclosures. In some of these embodiments, the method also includes determining a first composite index of the plurality of composite indices based on weighted scores of values of a first set of contributing metrics and determining a second composite index of the plurality of composite indices based on weighted scores of values of a second set of contributing metrics, wherein the first set of contributing metrics and the second set of contributing metrics includes sensor data from the plurality of sensors. In one example of these embodiments, the first set of contributing metrics overlaps with the second set of contributing metrics. In another example, the first set of contributing metrics only partially overlaps with the second set of contributing metrics. In another example, the method also includes dynamically adjusting one or both of the first set of contributing metrics and the second set of contributing metrics based on availability of sensor data from the plurality of sensors. In another example, the method also includes determining a third composite index based on weighted scores of values of a third set of contributing metrics. In one instance, the third set of contributing metrics does not overlap with the first set of contributing metrics or the second set of contributing metrics.

The term “system” may encompass all kinds of apparatuses, devices, and machines for processing data, including, for the purpose of illustration rather than limitation, a programmable processor, a computer, or multiple processors or computers. A processing system may include special purpose logic circuitry, e.g., an FPGA (field programmable gate array) or an ASIC (application specific integrated circuit). A processing system may include, in addition to hardware, code that creates an execution environment for the computer program in question, e.g., code that constitutes processor firmware, a protocol stack, a database management system, an operating system, or a combination of one or more of them.

A computer program (which may also be referred to or described as a program, software, a software application, an engine, a pipeline, a module, a software module, a script, or code) can be written in any form of programming language, including compiled or interpreted languages, or declarative or procedural languages, and it can be deployed in any form, including as a stand-alone program or as a module, component, sub-routine, or other unit suitable for use in a computing environment. A computer program may, but need not, correspond to a file in a file system. A program may be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program may also be deployed to be executed on a single computer or on multiple computers that, for example, are located at one site or distributed across multiple sites and interconnected by a communication network.

The processes and logic flows described in this specification can be performed by one or more programmable computers executing one or more computer programs to perform functions by operating on input data and generating output. The processes and logic flows can also be performed by, and apparatus can also be implemented as, special purpose logic circuitry, e.g., an FPGA (field programmable gate array) or an ASIC (application specific integrated circuit).

Computers suitable for the execution of a computer program can include, by way of example, general or special purpose microprocessors or both, or any other kind of central processing unit. Generally, a central processing unit will receive instructions and data from a read-only memory or a random access memory or both. A computer generally includes a central processing unit for performing or executing instructions and one or more memory devices for storing instructions and data. Generally, a computer will also include, or be operatively coupled to receive data from and/or transfer data to, one or more mass storage devices for storing data, e.g., magnetic, magneto-optical disks, or optical disks. However, a computer need not have such devices. Moreover, a computer may be embedded in another device, e.g., a mobile or cellular telephone, a personal digital assistant (PDA), a mobile audio or video player, a game console, a Global Positioning System (GPS) receiver, or a portable storage device (e.g., a universal serial bus (USB) flash drive), to name just a few.

Computer readable media suitable for storing computer program instructions and data include all forms of nonvolatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in, special purpose logic circuitry.

To provide for interaction with a user, embodiments of the subject matter described in this specification may be implemented on a computer having a display device, e.g., a CRT (cathode ray tube) or LCD (liquid crystal display) monitor, for displaying information to the user, as well as a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending Web pages to a Web browser on a user's user device in response to requests received from the Web browser.

Embodiments of the subject matter described in this specification can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the subject matter described in this specification, or any combination of one or more such back end, middleware, or front end components. The components of the system can be interconnected by any form or medium of digital data communication, e.g., a communication network. Examples of communication networks include a local area network (“LAN”) and a wide area network (“WAN”), e.g., the Internet.

Although this specification contains many specific implementation details, these details should not be construed as limitations on the scope of what may be claimed, but rather as descriptions of features that may be specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In certain implementations, multitasking and parallel processing may be advantageous. Other steps or stages may be provided, or steps or stages may be eliminated, from the described processes. Accordingly, other implementations are within the scope of the following claims.

Terms such as “a”, “an” and “the” are not intended to refer to only a singular entity but include the general class of which a specific example may be used for illustration. The terminology herein is used to describe specific embodiments, but their usage does not delimit.

When ranges are mentioned, the ranges are meant to be inclusive, unless otherwise specified. For example, a range between value 1 and value 2 is meant to be inclusive and include value 1 and value 2. The inclusive range will span any value from about value 1 to about value 2. The term “adjacent” or “adjacent to,” as used herein, includes next to,” “adjoining,” “in contact with,” and “in proximity to.”

As used herein, including in the claims, the conjunction “and/or” in a phrase such as “including X, Y, and/or Z”, refers to in inclusion of any combination or plurality of X, Y, and Z. For example, such phrase is meant to include X. For example, such phrase is meant to include Y. For example, such phrase is meant to include Z. For example, such phrase is meant to include X and Y. For example, such phrase is meant to include X and Z. For example, such phrase is meant to include Y and Z. For example, such phrase is meant to include a plurality of Xs. For example, such phrase is meant to include a plurality of Ys. For example, such phrase is meant to include a plurality of Zs. For example, such phrase is meant to include a plurality of Xs and a plurality of Ys. For example, such phrase is meant to include a plurality of Xs and a plurality of Zs. For example, such phrase is meant to include a plurality of Ys and a plurality of Zs. For example, such phrase is meant to include a plurality of Xs and Y. For example, such phrase is meant to include a plurality of Xs and Z. For example, such phrase is meant to include a plurality of Ys and Z. For example, such phrase is meant to include X and a plurality of Ys. For example, such phrase is meant to include X and a plurality of Zs. For example, such phrase is meant to include Y and a plurality of Zs. The conjunction “and/or” is meant to have the same effect as the phrase “X, Y, Z, or any combination or plurality thereof.” The conjunction “and/or” is meant to have the same effect as the phrase “one or more X, Y, Z, or any combination thereof.”

The term “operatively coupled” or “operatively connected” refers to a first element (e.g., mechanism) that is coupled (e.g., connected) to a second element, to allow the intended operation of the second and/or first element. The coupling may comprise physical or non-physical coupling (e.g., communicative coupling). The non-physical coupling may comprise signal-induced coupling (e.g., wireless coupling). Coupled can include physical coupling (e.g., physically connected), or non-physical coupling (e.g., via wireless communication). Operatively coupled may comprise communicatively coupled.

An element (e.g., mechanism) that is “configured to” perform a function includes a structural feature that causes the element to perform this function. A structural feature may include an electrical feature, such as a circuitry or a circuit element. A structural feature may include an actuator. A structural feature may include a circuitry (e.g., comprising electrical or optical circuitry). Electrical circuitry may comprise one or more wires. The electrical circuitry may be configured to coupe to an electrical power source (e.g., to the electrical grid). For example, the electrical circuitry may comprise a socket. Optical circuitry may comprise at least one optical element (e.g., beam splitter, mirror, lens and/or optical fiber). A structural feature may include a mechanical feature. A mechanical feature may comprise a latch, a spring, a closure, a hinge, a chassis, a support, a fastener, or a cantilever, and so forth. Performing the function may comprise utilizing a logical feature. A logical feature may include programming instructions. Programming instructions may be executable by at least one processor. Programming instructions may be stored or encoded on a medium accessible by one or more processors. Additionally, in the following description, the phrases “operable to,” “adapted to,” “configured to,” “designed to,” “programmed to,” or “capable of” may be used interchangeably where appropriate.

Modifications, additions, or omissions may be made to any of the above-described implementations without departing from the scope of the disclosure. Any of the implementations described above may include more, fewer, or other features without departing from the scope of the disclosure. Additionally, the steps of described features may be performed in any suitable order without departing from the scope of the disclosure. Also, one or more features from any implementation may be combined with one or more features of any other implementation without departing from the scope of the disclosure. The components of any implementation may be integrated or separated according to particular needs without departing from the scope of the disclosure.

It should be understood that certain aspects described above can be implemented in the form of logic using computer software in a modular or integrated manner. Based on the disclosure and teachings provided herein, a person of ordinary skill in the art will know and appreciate other ways and/or methods to implement the aspects using hardware and a combination of hardware and software.

Any of the software components or functions described in this application, may be implemented as software code using any suitable computer language and/or computational software such as, for example, Java, C, C#, C++ or Python, LabVIEW, Mathematica, or other suitable language/computational software, including low level code, including code written for field programmable gate arrays, for example in VHDL. The code may include software libraries for functions like data acquisition and control, motion control, image acquisition and display, etc. Some or all of the code may also run on a personal computer, single board computer, embedded controller, microcontroller, digital signal processor, field programmable gate array and/or any combination thereof or any similar computation device and/or logic device(s). The software code may be stored as a series of instructions, or commands on a CRM such as a random access memory (RAM), a read only memory (ROM), a magnetic medium such as a hard-drive or a floppy disk, or an optical medium such as a CD-ROM, or solid stage storage such as a solid state hard drive or removable flash memory device or any suitable storage device. Any such CRM may reside on or within a single computational apparatus, and may be present on or within different computational apparatuses within a system or network. Although the foregoing disclosed implementations have been described in some detail to facilitate understanding, the described implementations are to be considered illustrative and not limiting. It will be apparent to one of ordinary skill in the art that certain changes and modifications can be practiced within the scope of the appended claims.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g. “such as”) provided with respect to certain implementations herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or implementations of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

What is claimed is:

1. A system, comprising:
at least one controller for controlling one or more building systems based on a plurality of composite indices of one or more enclosures, the at least one controller configured to determine the plurality of composite indices based at least in part on sensor data from a plurality of sensors.

2. The system of claim 1, wherein:
the plurality of composite indices includes:
a safety index,
a health index, and
a performance index.

3. The system of claim 2, wherein the one or more enclosures comprise a building in which, or at which, the plurality of sensors is disposed and/or a space in the building.

4. The system of claim 1, wherein the plurality of sensors comprises a sensor ensemble comprising two or more environmental sensors.

5. The system of claim 1, wherein the plurality of composite indices is determined based also at least in part on data from one or more of a survey, a questionnaire, a work order, or user input.

6. The system of claim 1, wherein at least one of the composite indices is determined based on weighted scores of values of contributing metrics including the sensor data from the plurality of sensors.

7. The system of claim 1, wherein:
the plurality of composite indices is determined based on weighted scores of a plurality of subgroup indices, and
the weighted score of each subgroup index is determined based on weighted scores of values of contributing metrics including the sensor data from the plurality of sensors.

8. The system of claim 1, wherein:
the at least one controller is further configured to:
determine a first composite index of the plurality of composite indices based on weighted scores of values of a first set of contributing metrics and
determine a second composite index of the plurality of composite indices based on weighted scores of values of a second set of contributing metrics, and
the first set of contributing metrics and the second set of contributing metrics include the sensor data from the plurality of sensors.

9. The system of claim 1, wherein the at least one controller is further configured to:
determine whether data for a set of contributing metrics is available;
if data for a contributing metric of the set of contributing metrics is not available, remove the contributing metric from the set of contributing metrics and adjust each weighting factor for each remaining contributing metric in the set of contributing metrics; and
determine at least one of the composite indices based on the remaining contributing metrics and corresponding adjusted weighting factors.

10. The system of claim 1, wherein the at least one controller is further configured to determine availability of data for a minimum set of contributing metrics.

11. A method of controlling one or more building systems, the method comprising:
determining a plurality of composite indices of one or more enclosures based at least in part on sensor data from a plurality of sensors; and
controlling the one or more building systems based on the plurality of composite indices.

12. The method of claim 11, wherein:
the plurality of composite indices includes:
a safety index,
a health index, and
a performance index of the one or more enclosures.

13. The method of claim 12, wherein the one or more enclosures comprise a building in which, or at which, the plurality of sensors is disposed and/or a space in the building.

14. The method of claim 11, wherein the plurality of sensors comprises a sensor ensemble comprising two or more environmental sensors.

15. The method of claim 11, wherein the determination of the plurality of composite indices is also based at least in part on data from one or more of a survey, a questionnaire, a work order, or user input.

16. The method of claim 11, further comprising determining the plurality of composite indices based on weighted scores of values of contributing metrics including the sensor data from the plurality of sensors.

17. The method of claim 11, further comprising:
determining a weighted score for each contributing metric associated with the plurality of composite indices;
determining a weighted score for each subgroup index of a plurality of subgroup indices by combining weighted scores of a set of contributing metrics associated with the subgroup index; and
determining each composite index of the plurality of composite indices by combining weighted.

18. The method of claim 11, further comprising sending a notification of the one or more actions to one or more occupants of the one or more enclosures in which, or at which, the plurality of sensors is disposed.

19. The method of claim 11, further comprising sending the notification to a digital twin of the one or more enclosures.

20. A system, comprising:
a plurality of sensors at a building and configured to generate sensor data associated with an environment in the building;
at least one controller configured to:
receive the sensor data, and
determine, based at least in part on the sensor data, a first composite index and a second composite index of a space of the building; and
a user interface configured to present information associated with the first composite index and the second composite index.
